(19) United States
(12) Patent Application Publication
(10) Pub. No.: US 2024/0411326 A1
(43) Pub. Date: Dec. 12, 2024

(54) METHOD AND SYSTEM FOR GENERATING A RADIATION PLAN

(71) Applicant: Számítástechnikai És Automatizálási Kutatóintézet, Budapest (HU)

(72) Inventors: Barnabás Bugár-Mészáros, Budapest (HU); András Majdik, Budapest (HU); Zoltán Rózsa, Gyömro (HU); Tamas Szirányi, Budapest (HU)

(21) Appl. No.: 18/714,473

(22) PCT Filed: May 27, 2022
(86) PCT No.: PCT/HU2022/050058

§ 371 Date: May 29, 2024

(30) Foreign Application Priority Data
Jul. 27, 2021 (HU) P2100206

(51) Int. Cl. G05D 1/689; A61L 2/10; G05D 105/10; G05D 111/10

(52) U.S. Cl. CPC G05D 1/689; A61L 2/10; A61L 2202/11; A61L 2202/14; G05D 2105/10; G05D 2111/10

(57) ABSTRACT

The invention is a method for generating a radiation plan of a scene in particular for a disinfection robot adapted for radiating disinfection light, in the course which
providing or generating a 3D model (136) with 3D elements,
generating radiation plan candidates (112) for the first 3D model (136) based on artificial intelligence, each plan candidate (112) comprising one or more radiation position and respective radiation time,
generating one or more respective irradiance ratio estimation (134) of the 3D model (136) for the plan candidates (112), and
obtaining the radiation plan (135) from the plan candidates (112) based on the one or more respective irradiance ratio estimation (134) by means of the first radiation plan generator module (130).

The invention is, furthermore, a system for generating a radiation plan.

### TECHNICAL FIELD

The invention relates to methods and systems for generating a radiation plan of a scene in particular for a disinfection robot adapted for radiating disinfection light.

### DESCRIPTION OF PRIOR ART

The autonomous cleaning of various private and public buildings by intelligent robots has become very popular recently. The machines which were created to perform ordinary tasks such as vacuum-cleaning are mostly limited to ground operation. However, the proper disinfection of vertical surfaces and all kinds of objects is also required in hospitals, offices and in other frequently visited places. This challenge can be tackled by using UV-C light which is proven to be very successful in reducing the number of bacteria and viruses.

The proper disinfection of indoor places shared by a group of people on a daily basis has become a fundamental task recently. This problem is usually tackled by using either chemical-based methods or UV-C light. The number of mobile robots equipped with UV-C tubes has increased in the past few years due to their ability to effectively disinfect large buildings without supervision (a sketch showing the structure of such a mobile robot 340 can be seen in FIG. 21). By mounting UV-C tubes on a moving platform, high quality surface and air disinfection can be achieved without having to supervise the process. The light source can be either lamp or LED, with the former being cheaper, while the latter is more compact and energy-saving. The effectiveness of the disinfection is affected by many factors including temperature, humidity, room configuration and lamp placement.

In CN 111905135 A a disinfection device using a sprayer for disinfecting is disclosed. The disinfection device is based on an unmanned artificial intelligence robot, which can simulate the distribution of the target space.

Furthermore, in WO 2021/026649 A1 a system and method of semi-autonomous cleaning of surfaces is disclosed.

In US 2021/0089040 A1 an obstacle recognition method for autonomous robots is disclosed.

In view of the known approaches, there is a demand for an approach applicable for disinfection robots by the help of which disinfection may be of higher efficiency than in the known approaches.

### DISCLOSURE OF THE INVENTION

The primary object of the invention is to provide a method and a system for generating a radiation plan which are free of the disadvantages of prior art approaches to the greatest possible extent.

The object of the invention is to provide a method and a system for generating a radiation plan which is suitable for using in disinfection robots having higher efficiency than the known approaches.

The objects of the invention can be achieved by the method for generating a radiation plan according to claim 1 and claim 17, as well as by the corresponding system according to claim 16 and claim 18, respectively. Preferred embodiments of the invention are defined in the dependent claims.

The invention is thus a method (procedure) and system for generating a radiation plan suitable for using in—preferably autonomous—disinfection robots. In the invention the irradiation process is simulated (e.g. by the disinfection robots) by computing irradiance on nearby surfaces of a scene. Thereby, the irradiation procedure is implemented for a disinfection robot.

### BRIEF DESCRIPTION OF THE DRAWINGS

Preferred embodiments of the invention are described below by way of example with reference to the following drawings, where

FIG. 1A is a diagram illustrating the method and system according to the invention,

FIG. 1B is a diagram illustrating an embodiment of the method and system according to the invention,

FIG. 2 is a flowchart illustrating the environment reconstruction in an embodiment,

FIG. 3 is a flowchart illustrating irradiance evaluation in an embodiment,

FIGS. 4A and 4B are flowcharts illustrating the direct and indirect irradiance estimation respectively,

FIG. 5 is a flowchart illustrating irradiance plan generation according to various embodiments of the invention,

FIG. 6 is a block diagram illustrating the hierarchy of algorithms applicable in the framework of the invention,

FIG. 7A is a flowchart illustrating an embodiment based on evolutionary algorithm,

FIG. 7B is a flowchart illustrating a further embodiment based on evolutionary algorithm,

FIG. 8 is a diagram illustrating the recombination in an embodiment based on genetic algorithm,

FIG. 9A is a schematic drawing showing a room for illustrating modification of positions in an embodiment based on genetic algorithm,

FIG. 9B shows a detail of FIG. 9A in somewhat different representation,

FIG. 9C is a drawing for illustrating a room with general shape,

FIG. 10A is a flowchart illustrating an embodiment based on incremental path generation,

FIG. 10B is a drawing of a room for illustrating the operation of incremental path generation algorithm,

FIG. 11A is a flowchart illustrating an embodiment based on reinforcement learning,

FIG. 11B is shows a room for illustrating the embodiment based on reinforcement learning,

FIG. 12A shows a further embodiment based on deep learning,

FIG. 12B is a schematic drawing of a room illustrating the deep learning algorithm,

FIG. 13 is a flowchart illustrating an embodiment of the environment reconstruction,

FIG. 14 is a flowchart illustrating an embodiment based on genetic algorithm,

FIGS. 15A-15C are schematic drawings for illustrating visibility parameter,

FIG. 16 is a schematic drawing for illustrating the indirect irradiance,

FIG. 17 is a schematic drawing for illustrating the inverse square law for distance,

FIG. 18 is an illustration of irradiation,

FIG. 19A an illustration for a 3D model,

FIGS. 19B-19H illustrate how the irradiance is built up,

FIG. 20A is a further illustration of a 3D model,

FIGS. 20B-20E are illustration for different factors of irradiation (the 3D model of FIG. 20A),

FIG. 21 illustrates an exemplary robot,

FIG. 22 illustrates detail of an equipment used in experiments,

FIGS. 23A and 23B are illustrations for handling a room part,

FIGS. 24A-24C are illustrations for different factors of the irradiation,

FIGS. 25A-25D are further illustrations for different factors for irradiation

FIGS. 26 and 27 are diagrams for showing different aspects of irradiance, and

FIG. 28 is a schematic drawing for illustrating an experimental measurement.

### MODES FOR CARRYING OUT THE INVENTION

The invention is a method for generating a radiation plan of a scene (called first scene for the herebelow presented method according to the invention) in particular for a disinfection robot adapted for radiating disinfection light (generally for radiating disinfection radiation/irradiation, see below).

The disinfection robot is generally a disinfection device, which is not necessary to be a robot, since the main aspect of the invention is to generate (determine) a radiation plan, which is applicable not only for a robot (being an autonomous wheeled machine) but it can be an input for any other wheeled machine which may be controlled in any other way, e.g. manually. Moreover, the disinfection light is typically UV-C light but other types of treatment are conceivable for disinfection, such as gamma irradiation treatment and heat treatment. Accordingly, in general disinfection radiation/irradiation (of light or other type of radiation) is applied in the framework of the invention.

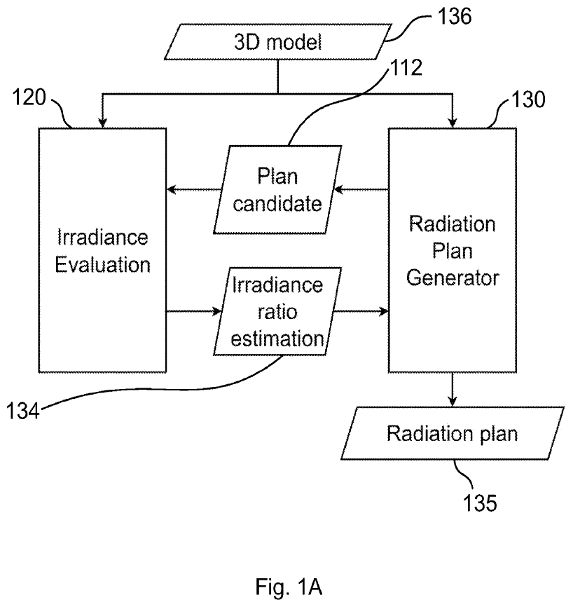

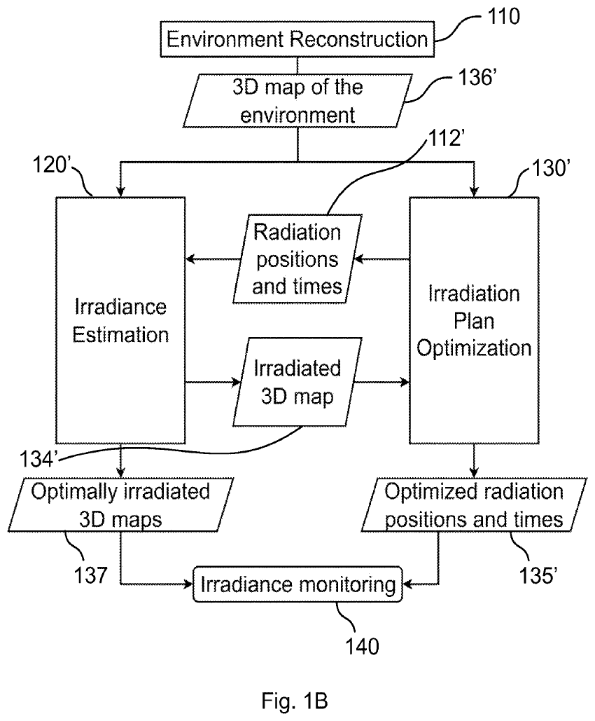

The schematic outline of the method is illustrated in FIG. 1A, accordingly firstly the aspects of the invention are introduced referring to FIG. 1A. It is noted that FIG. 1A is also an illustration of the system according to the invention.

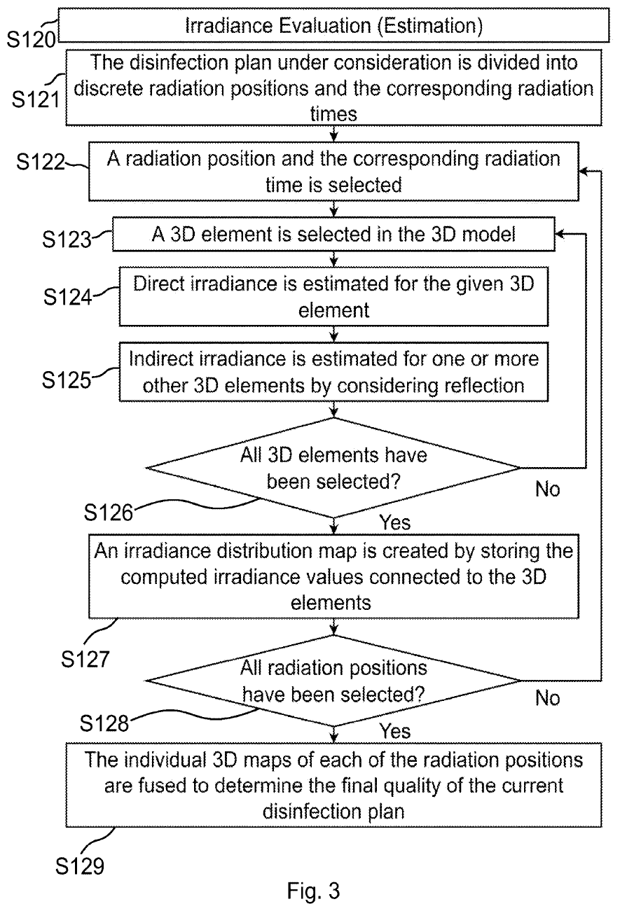

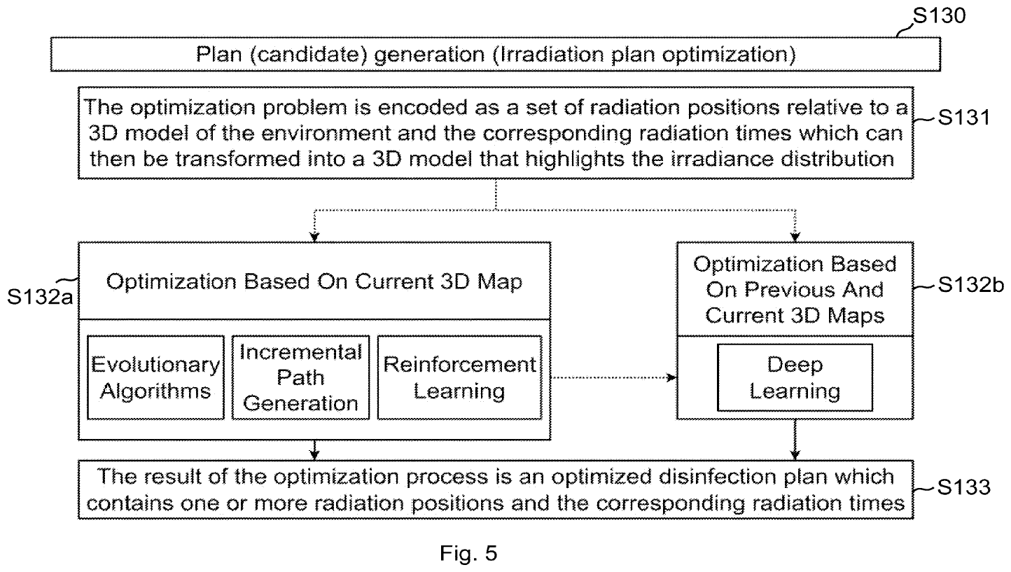

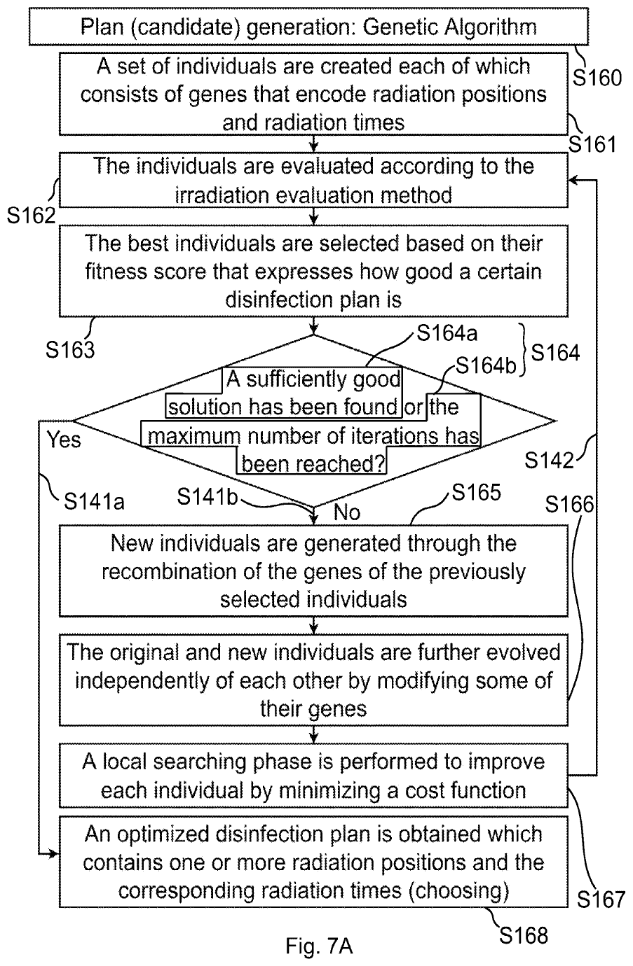

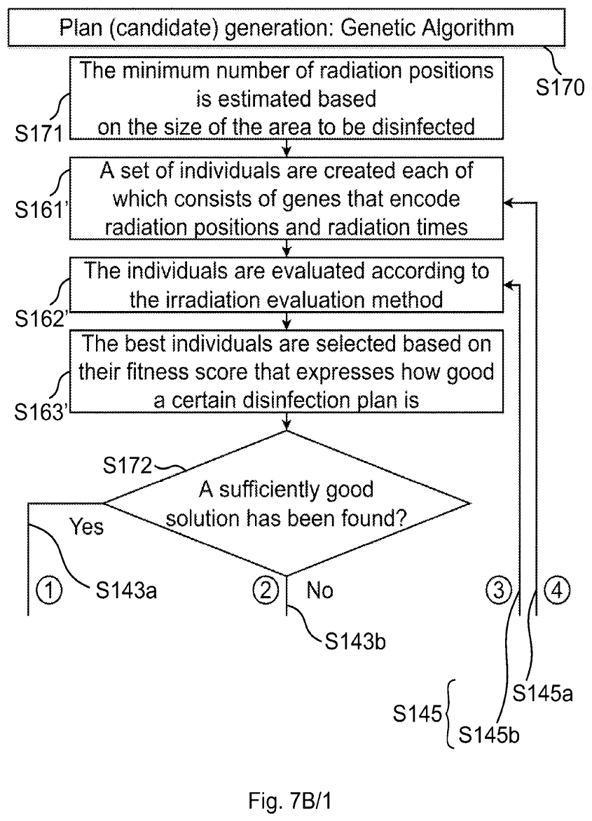

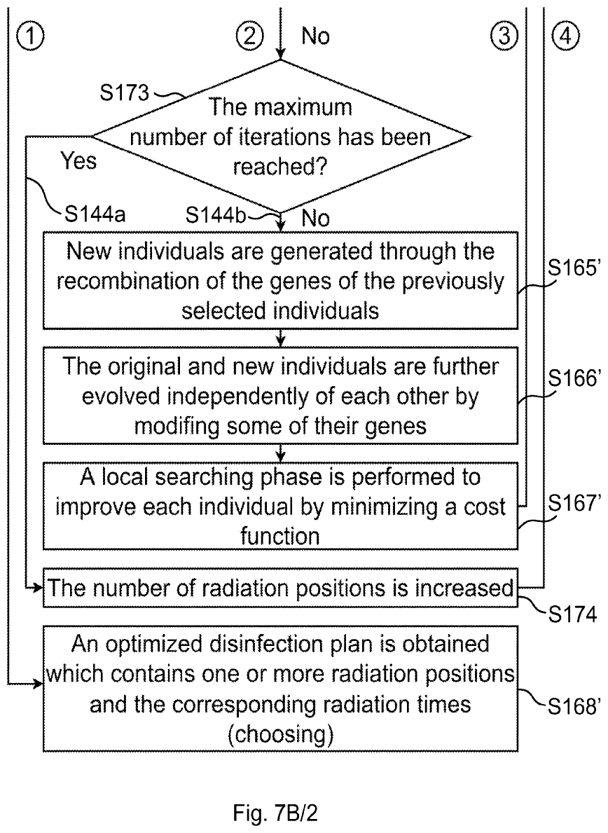

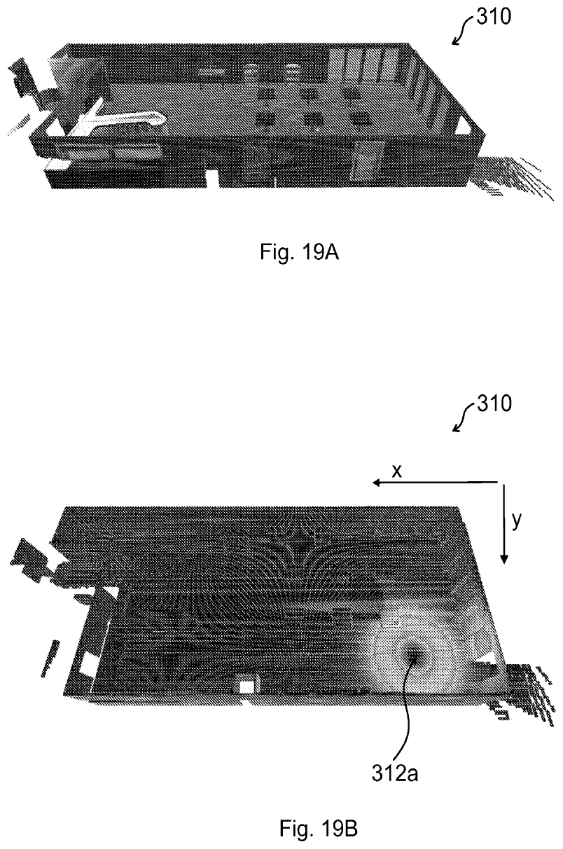

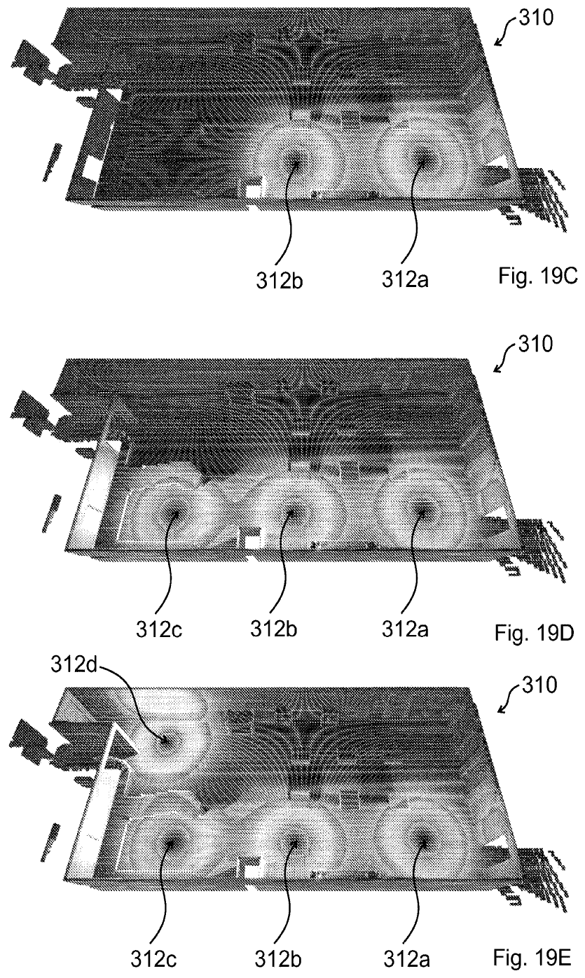

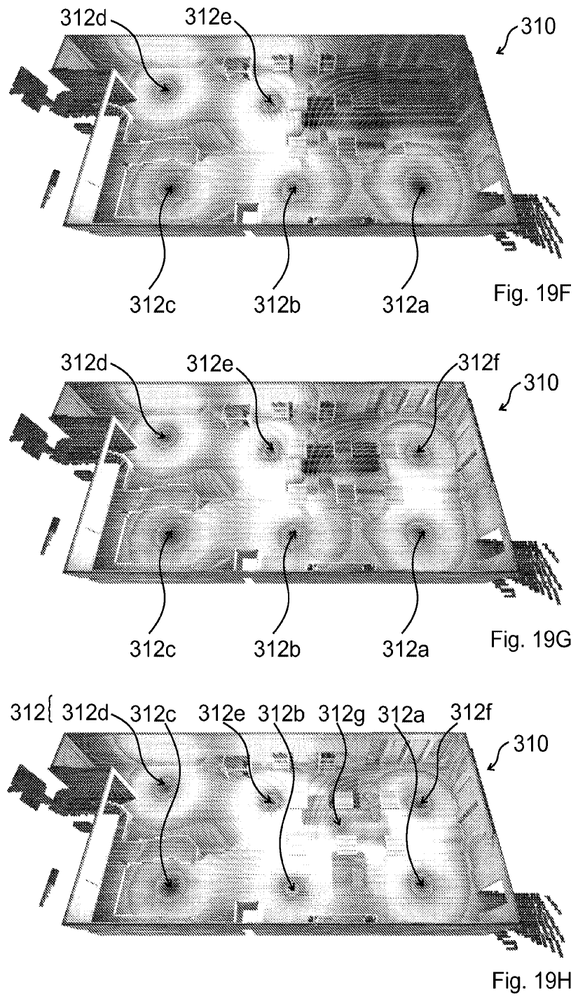

In the course of the method according to the invention:
a first discretized 3D model with a plurality of 3D elements of a first scene is provided (available) or generated (see FIG. 1A for 3D model 136 where it is not specified whether it is generated or provided; see also FIG. 19A for an exemplary 3D model 310), and
in a plan candidate generating step (see operational step S130 in FIG. 5 for an embodiment) one or more plan candidate (see a plan candidate 112 in FIG. 1A; it can also be called as radiation plan candidate) of the radiation plan (see a resulted radiation plan 135 in FIG. 1A) by means of a first radiation plan generator module (unit; see a radiation plan generator module 130 in FIG. 1A) based on artificial intelligence, wherein each of the one or more plan candidate comprising (having)
   one or more radiation position and
   one or more respective radiation time each corresponding to a radiation position,
   wherein a radiation position and a corresponding radiation time constitute a radiation data pair,
in an irradiation evaluation step (see operational step S120 in FIG. 3 for an embodiment) one or more respective irradiance ratio estimation (see an irradiance ratio estimation 134 in FIG. 1A) of the first 3D model for the one or more plan candidate by evaluating the one or more plan candidate by means of irradiation evaluation module (see an irradiation evaluation module 120 in FIG. 1A), and
the radiation plan is obtained (in this way, generated) from the one or more plan candidate based on the one or more respective irradiance ratio estimation by means of the first radiation plan generator module in a radiation plan choosing step (various corresponding operational steps S168, S168′, S197, S226 are shown in FIGS. 7A, 7B, 10A and 11A).

In the following, in connection with FIG. 1B some aspects of the invention will be further detailed and also some optional features will be given. Moreover, after the description of FIG. 1B details of the main steps/main modules will be given in respective embodiments.

Before these disclosures, however, it is introduced that some embodiments of the invention relate to a system for generating a radiation plan of a scene (it can also be interpreted based on FIG. 1A).

The system according to the invention comprises
a first radiation plan generator module (for the radiation plan generator module 130, see FIG. 1A) based on artificial intelligence and adapted for generating one or more plan candidate of the radiation plan for the first 3D model with a plurality of 3D elements of a first scene, wherein each of the one or more plan candidate comprising
   one or more radiation position, and
   one or more respective radiation time each corresponding to a radiation position
   wherein a radiation position and a corresponding radiation time constitute a radiation data pair, and
an irradiation evaluation module (for the irradiation evaluation module 120, see FIG. 1A) adapted for generating a respective irradiance ratio estimation of the first 3D model by evaluating the one or more plan candidate,
   wherein the first radiation plan generator module is furthermore adapted for obtaining the radiation plan from the one or more plan candidate based on the one or more respective irradiance ratio estimation.

It is meant on generating the one or more plan candidate that one or more position and corresponding radiation times, i.e. the one or more radiation data pair is determined by the first radiation plan generator module (being based on artificial intelligence). Furthermore similarly, the one or more radiation data pair (i.e. both the radiation position(s) and radiation time(s), not only one of them) obtained by first radiation plan generator module is utilized in the irradiation evaluation module. The ‘radiation data pair’ term shows that it contains a position and a time, i.e. a pair. It may also be called ‘pair of radiation position and radiation time’ or ‘optimizable data (pair/piece)’.

According to the invention, the radiation plan is generated for a 3D model which is readily available or—also readily—generated as an input for the method. It is noted that in this latter case, which is an embodiment, the generation of the 3D model can be considered as the part of the invention, i.e. can be considered as an additional step to the invention.

Accordingly, the 3D model constitutes an input for the method according to the invention. Furthermore, a 3D model can be generated from a real scene as well as virtual scene by means of one or more real sensor and one or more virtual sensor, respectively. Note that in this way a radiation plan can be obtained for real and virtual scenes as well (this is irrelevant from the point of view of the 3D model and the radiation plan), the relevant is to have a 3D model as an input for the method and system according to the invention.

This flexibility is advantageous, since in this way the method and system according to the invention can be tested in virtual environment. Moreover, this is also advantageous for the embodiment applying deep learning, since it is preferred that a huge amount of virtual data may be available for training.

The radiation positions are independent of the 3D model in the sense that no 3D element belongs to the radiation position directly. Therefore, only the relative positions between the voxels and the light source are considered (for the positions a coordinate system can be defined, see also Table 1 and FIG. 19B). In the 2DoF (degree of freedom) case the coordinates describe the x-y position of the centre of the lamp and its height is constant in all positions which can computed based on the structure of the robot (in the experiments it is an exemplary value of 1.65 m). If 6DoF were considered then in addition to the x-y-z coordinates of the centre of the light source, the orientation of it was also considered (orientation can be taken into account in the lamp characteristics as given below).

Both the first radiation plan generator module and the irradiation evaluation module have access to the 3D model, the former determines the radiation plan or radiation plan candidates within the framework of the 3D model (i.e. the positions are defined relatively to the 3D model), the latter handles the irradiance of the 3D elements thereof.

In connection with the radiation position, the followings are noted. On the one hand, the radiation point can be represented in 2D or in 3D based on whether what kind of lamp is used (e.g. it has a well-defined position or the lamp is fixed to the end of a robotic arm). Alternatively, the radiation position may be called as radiation point or radiation location.

Preferably, also the orientation of the light source can also be considered, then the optimization problem is extended so that each radiation data pair comprises the pose (i.e. position and orientation) of the light source and the corresponding radiation time. Consequently, in this case the radiation plan candidates will comprise 7n parameters (n is the number of radiation data pairs) where position and orientation are described by three coordinates and three angles respectively. After the encoding of the plan candidates is extended, the described optimization methods can be used the same as when only 2D/3D coordinates are considered.

The disinfection robot is preferably adapted for radiating disinfection light by means of a lighting device. This lighting device is mentioned as lamp throughout the description.

Moreover, the lighting device/light source/lamp has a lighting device/light source/lamp characteristics (e.g. I(α(x)), see the description of FIG. 18). It is to be understood that the lamp characteristics is an input for the method according to the invention, i.e. for the optimization and the irradiance evaluation. It is taken into account in the steps of the method according to the invention to obtain the radiation plan with the radiation positions and the corresponding radiation times. Accordingly, the radiation plan is built with a specific lamp characteristics which has an impact on the distribution of the one or more radiation position and respective one or more radiation time.

The characteristics of the light source serves as an input to the created system. Consequently, many types of light source system can be used which may contain one or more lamps and/or LEDs of the same or different type. Then the whole characteristics of this light source system has to be defined and given to the created system as input. Although multiple lamps/LEDs can be used, hereafter the document will refer to the light source system as light source or lamp.

In many of the embodiments, obtaining radiation plan in the radiation plan choosing step is—on the top of the one or more irradiance ratio estimation—based on a total radiation time obtained for each plan candidate by summing up the corresponding one or more radiation time for all of the one or more radiation position (e.g. in the reinforcement learning based approach this is preferably but optionally applied).

Furthermore, the above operation in the radiation plan choosing step can be detailed with that the one or more respective irradiance ratio estimation is forwarded to the first radiation plan generator module from the irradiation evaluation module. Thus, it also may be considered at least one cycle of iteration with the parameters is performed.

An embodiment being similar to that of FIG. 1A is illustrated in FIG. 1B.

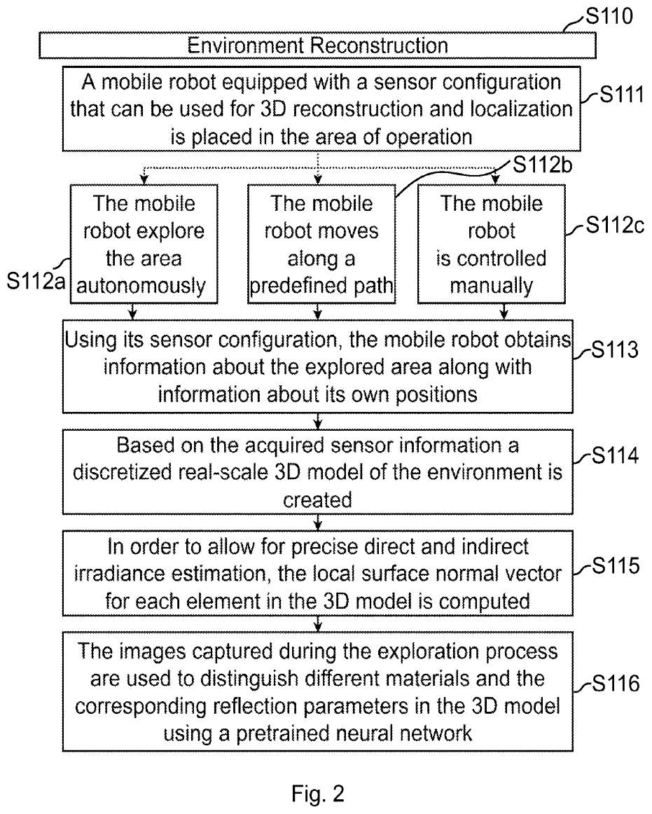

In many cases, the first step of the creation of an optimal disinfection plan is to reconstruct the environment to be disinfected. This can be done in environment (scene) reconstruction module 110 in FIG. 1B, as a result of which a 3D map of the environment 136′ is obtained (environment is meant as an alternative word of scene in this context; a room also can be a scene, see below). The detailed description of this process is described by FIG. 2 in an embodiment (see below). The output of this stage is the discretized real-scale 3D model of the area of operation (scene) which serves as a basis for irradiance estimation and optimization.

The computation of the optimal disinfection plan is performed by two modules that closely rely on each other. An irradiance estimation module 120′ (which is the realization of an irradiance evaluation module in an embodiment) provides a method for computing the irradiance distribution on the input 3D model assuming that a set of radiation positions and times 112′ are provided. The computations are performed according to the Irradiance Estimation Formula (see below for a preferred formula).

An irradiation plan optimization module (block; which is the realization of radiation plan generator module in an embodiment) is responsible for creating possible radiation (disinfection) plans, i.e. radiation—one or more—positions and times and for evolving them through different optimization algorithms.

The word optimize, as well as optimization is utilized in connection with the radiation plan generator module based on artificial intelligence (i.e. stochastic or learning-based within the framework of the invention). Thus, in the context of the present description, these words are also meant to an “obtaining” process which has this basis. Accordingly, these words have an extra meaning in this context upon their ordinary meanings.

The irradiation plan optimization module 130′ creates one or more possible disinfection plans, i.e. (radiation) plan candidates and feeds them into the irradiance estimation module 120′ which provides the corresponding irradiated 3D maps which reflect how good the disinfection plans were (see below for details in connection with FIG. 3; and in connection with FIG. 5: when stochastic optimization or reinforcement learning is performed, the irradiation plan optimization module 130′ directly uses the feedback, the irradiated 3D maps, to obtain better possible solutions; further, in connection with FIG. 5, the deep-learning approach is slightly different in the sense that it uses the feedback to train a neural network, when the neural network is trained, it can provide a disinfection plan for a new 3D model without requiring iterative feedback).

It has to be noted, that while the iterative optimization—illustrated in FIG. 1B—is performed, only one irradiated 3D map (i.e. the final disinfection result considering the given radiation positions and times) per disinfection plan (candidate) is used. The outputs of these two modules (i.e. modules 120′ and 130′) are the optimized disinfection plan (optimized radiation positions and times 135′) and the corresponding 3D models which contain the real irradiance values for all 3D elements (i.e. optimally irradiated 3D maps 137).

Although during the optimization, only one 3D map per disinfection plan candidate is used, for visualization purposes multiple 3D maps can be obtained. These 3D maps can be acquired by splitting the final disinfection results according to radiation positions, radiation times or different factors that were considered during irradiance estimation.

The task of creating an optimal disinfection plan for an unknown environment is divided into three major parts. First, the discretized real-scale 3D model of the target environment is created (generated or provided). Preferably, the 3D model is an integral part of both direct and indirect irradiance estimation since most of the considered factors directly depend on the structure and type of the environment (see below for details). The environment reconstruction step is further detailed in FIG. 2 in an embodiment.

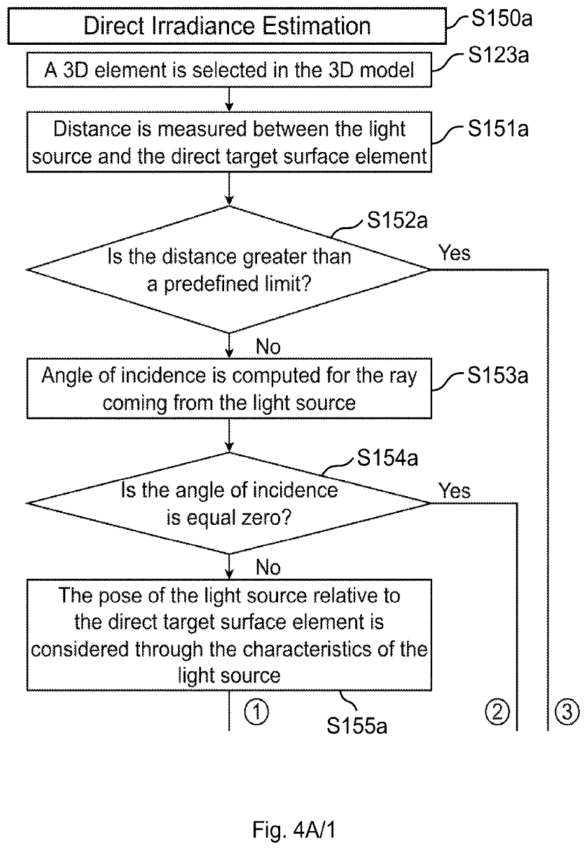

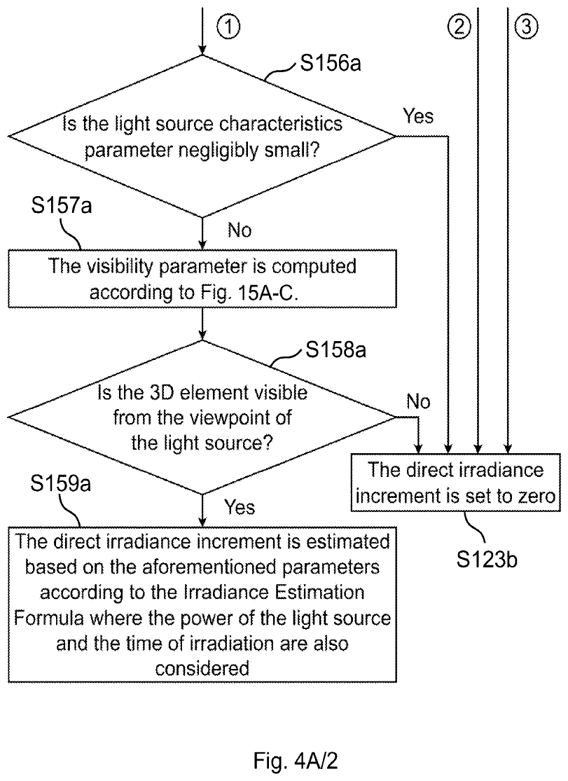

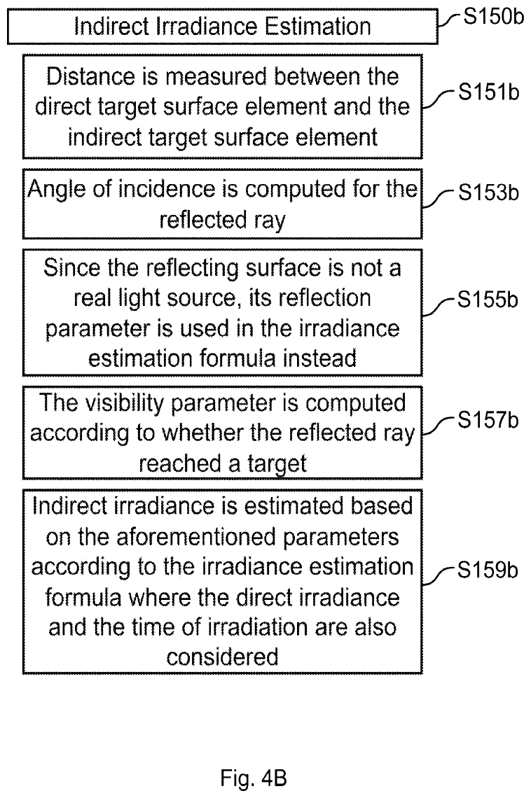

The second part is the irradiance estimation module 120′ that defines a method which can provide an irradiance distribution map based on the created 3D model and a possible disinfection plan that contains radiation (one or more) positions and times. This module is further detailed in FIG. 3 for an embodiment (see also FIG. 4A-4B).

Finally, since the 3D model of the environment and the irradiance evaluation method are available, an optimization module 130′ is used to find an optimal disinfection plan by applying either stochastic or learning-based methods.

The detailed description of various embodiments of optimization processes are shown in FIGS. 7A, 10A, 11A.

Once the calculations are finished, the irradiated 3D model can be visualized in an irradiance monitoring module 140 (with other terms, exposure monitoring module, visualization module) which shows the distribution of irradiance on the surfaces of the target environment. The role of this module is detailed below, many times its tasks are referred as visualisation (see among others, FIGS. 19A-19H, 20A-20E, 24A-24C, and 25A-25D).

In the obtained model, the user may for instance detect some areas that could not be reached by the UV-C rays anyway, or examine that from which radiation positions which surfaces were disinfected. These show why it is worth to calculate and even visualise the irradiance distribution. Sometimes, it can be observed in a visualisation why a remaining part cannot be irradiated or it can be also checked that which areas can be covered from each position.

In the following, the invention is introduced via exemplary embodiments to highlight some details, firstly in a nutshell. These details given below are many times optional in the invention.

The robot first preferably goes throughout the area of operation (called above scene or, elsewhere, environment) and creates a 3D model thereof, using the obtained sensor data. As shown elsewhere, the 3D model of scene can be obtained several ways.

Then, irradiance is computed considering several factors in connection with the UV-C lamp (preferably used for disinfection) and its position relative to the environment. Based on the computed values, the lamp positions are optimized using e.g. a genetic algorithm to improve the efficiency of disinfection. Lastly, the 3D model can be preferably illustrated showing the distribution of irradiance (it can be displayed, since the irradiance is registered for the discretized 3D model).

In the following some exemplary details are given about the environment reconstruction.

In order to estimate the irradiance in the environment of the autonomous robot, the 3D model of the surrounding objects has to be created, since most of the considered factors that affect the irradiance estimation depend on the position of the lamp relative to the irradiated surfaces. On the one hand, it is important to preserve as many details of the objects in the environment as possible to precisely estimate irradiance on their surfaces. On the other hand, as the time required for the calculation highly depends on the size of the 3D map, it must not have contained too many elements.

For this purpose, the rtabmap package (M. Labbe and F. Michaud, “Rtab-map as an open-source lidar and visual simultaneous localization and mapping library for large-scale and long-term online operation”, Journal of Field Robotics, vol. 36, no. 2, pp. 416-446, 2019) of ROS can be preferable applied which provides a very effective octree-based voxel representation using only a depth camera (A. Hornung et al.: “OctoMap: An efficient probabilistic 3D mapping framework based on octrees” Autonomous Robots, 34(3):189-206, 2013.; see also the details of OctoMap in this reference).

The main advantage of this representation is that the voxel grid can be applied as an occupancy grid which can be directly used to generate path between radiation positions and avoid obstacles. Furthermore, the fact that the 3D model is stored in a tree structure accelerates the irradiance calculations since each element can be accessed very quickly. Lastly, the structure allows for using raytracing efficiently which is essential in the computations (of visibility parameters and indirect irradiance).

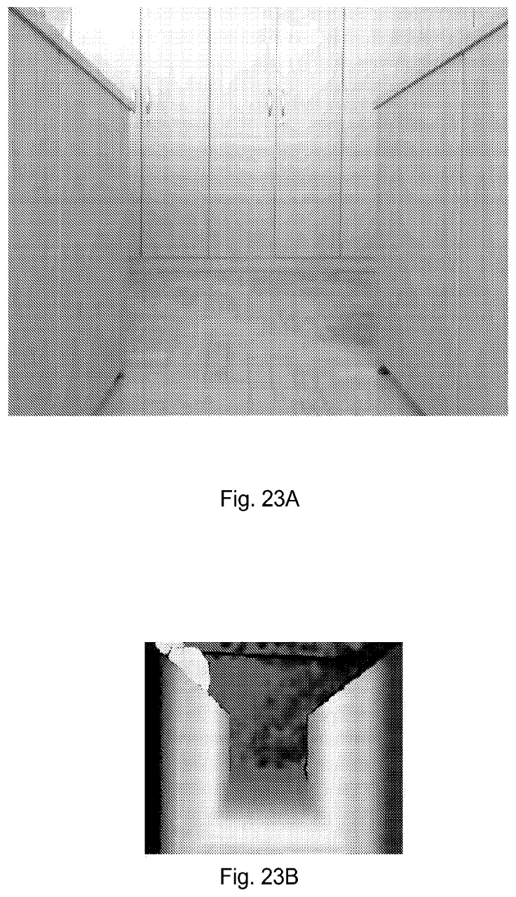

The RGB and depth images provided by the Intel RealSense d435i depth camera used in our experiments are shown in FIGS. 23A-23B. As the robot moves on its trajectory, the nearby objects and surfaces are reconstructed and the 3D model is updated. This is the realisation of making simultaneous localization and mapping (SLAM) in a closed, open or partly open territory (scene) without or with as minimal as possible human interaction.

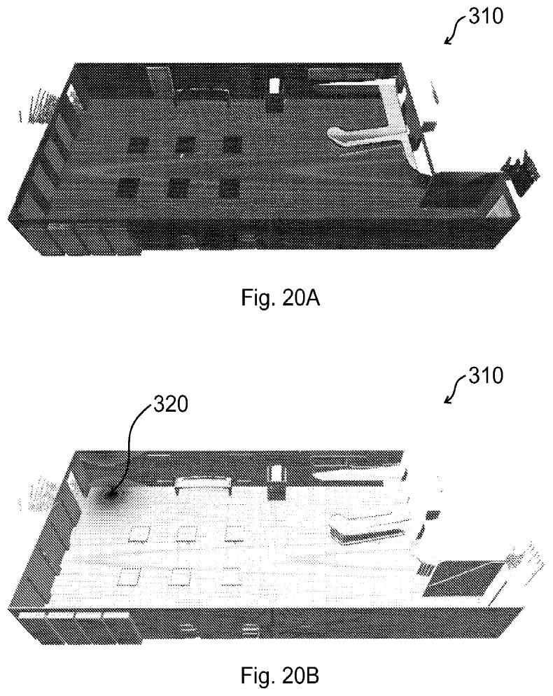

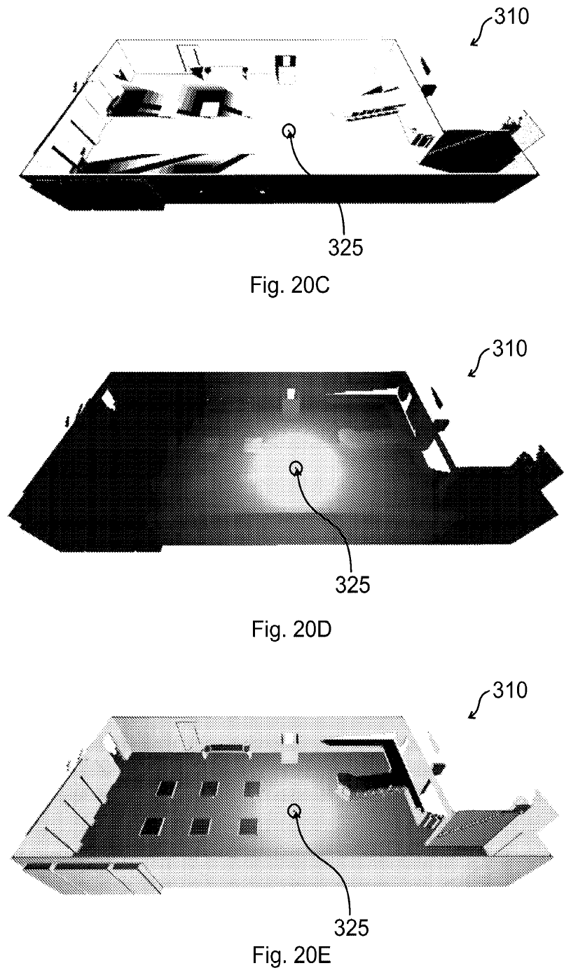

The reconstruction task was performed in our experiments using a virtual robot equipped with an RGB-D camera (see for an exemplary Waffle robot in https://emanual.robotis.com/docs/en/platform/turtlebot3/hardware_setup/) and the environment was represented as a 3D occupancy grid. FIG. 19A shows a resulting 3D model 310 of the primary test environment (see also description of FIGS. 19A-19H and 20A-20E).

As it can be seen in FIG. 19A, the mobile robot can only reconstruct those parts of the environment which can be directly perceived by the RGB-D camera. Since the test environment was reconstructed by a Waffle mobile robot which is relatively small (141 mm height), there are some missing parts in the 3D model. Such parts can be recognized near the bottom left corner of the image, where a smaller part of the wall was covered (and thus missing from the 3D model) by the desk between the robot and the mapped area. Similarly, a few missing elements can be observed near the cabinets in the room.

There were two minor difficulties with the created 3D model which were to be resolved.

Firstly, most of the walls were one voxel wide (built normally with single voxel width according to the data of the sensors exploring the scene). Consequently, if the colours of these cells were set according to the estimated irradiance, it could not be decided which side of the wall had been disinfected. In order to create surfaces that are at least two voxels wide, the map was first pruned and then expanded (i.e. the resolution of the map was set from 5 cm to 10 cm, then back to 5 cm). A minor drawback of this solution was that not all details of the objects could be preserved. Similar results could have been obtained, if the mapping was originally performed at 10 cm resolution. Secondly, on the sides of horizontal planes (e.g. tables) additional voxels were reconstructed which covered several cells that would have normally been reached by the UV-C light. This problem was partially solved by performing raycasting from multiple points of the lamp.

The main problem with one-voxel-wide surfaces is that if for instance a wall is present in the middle of a room then it cannot be decided which side of this wall was irradiated. It may happen that it was irradiated from one or both sides.

A further problem is that if the one-voxel-wide surface is irradiated from both sides then the computed irradiance values are accumulated but this value would not represent the real disinfection level on either side of the wall.

Similarly, to the secondly mentioned problem a one-voxel-wide horizontal surface, like a table, can also cause problems since it cannot be decided whether the top or bottom or both surfaces have been irradiated. Two solutions are proposed to this problem by leveraging that the 3D model is stored in a tree (more precisely in an octree) structure.

On the one hand, the size of the 3D elements can be halved thus there will be eight smaller elements instead of each original one (that is why it is called octree). This way minimum two-voxel-wide surfaces can be obtained but the computational demand of irradiance estimation would increase due to the increased number of elements.

On the other hand, each voxel (5 cm size, more generally between 1-20 cm size) or set of voxels (eight voxels form a larger voxel) can be replaced by a voxel with bigger size (a voxel from the higher level in the tree, i.e. the parent voxel—10 cm size) which can then be divided into eight smaller (child—5 cm size) voxels. Note that each parent voxel is created for which at least one child voxel is present. This way one-voxel-wide surfaces are thickened by at least one other layer resulting in at least two-voxel-wide surfaces. In this case, the computational demand will not increase that much, however, there will be parts in the 3D model that are marked as occupied although they were free in the real environment resulting in minor inaccuracies, which can be fully tolerated.

In simulated environments, the created rooms often do not have a ceiling. In the real world, this does not hold true obviously, however, the mobile robot which performs the 3D mapping of the place to be disinfected might not be able to reconstruct the ceiling, especially if an RGB-D camera is used. This follows from the fact that an RGB-D camera has a limited field of view and it usually points in the direction of the robot's movement. This way, the walls can be reconstructed, however since the camera usually does not move during the operation (relative to the robot) there might be missing elements near the top of the room.

On the one hand, the ceiling of the room is important because the irradiance values are preferred to be estimated on these surface elements as well (these are essentially surfaces of 3D elements). On the other hand, when reflected irradiance values are also calculated, the ceiling might serve as either a surface from which the UV-C rays are reflected, or a surface to which an UV-C ray is reflected.

In order to take these factors into consideration, the preferred algorithm extends the original 3D model of the environment by adding new elements which represent the ceiling. If the ceiling could be partially reconstructed then there will be elements at the height of the top of the walls which are floating thus can be considered as the elements of the ceiling. Since in this way the height of the ceiling is known it can be extended by inserting the missing elements. If the ceiling of the 3D model is completely missing, then the height of the room can be estimated by measuring the height of the walls. After that, for all elements of the floor (which is usually well reconstructed) a new element is inserted into the 3D model above them at the height of the estimated ceiling.

The optimization stage of the method and system according to the invention can also be improved, if more information is obtained on the environment. With the preferably used SLAM algorithm, a uniform 3D model can be created, i.e., the different materials—present in many cases in a scene—are not distinguished.

However, the type of various surfaces might affect the required irradiation time. Preferably, a new component can be added to the mapping algorithm by the help of which different surface types can be recognized, thus an additional weight can be assigned to each voxel in the obtained model. Also, it is also possible to manually set these weights based on prior knowledge. Therefore, the improvement achieved through the optimization could be even more significant.

Accordingly, in addition to their locations in the 3D space and their occupancy values, other meaningful information associated with the 3D elements (voxels) of the model can be stored. For instance, the colour values can be useful when the 3D model is visualized. The reflection and absorption parameters of the 3D elements of the 3D model are also important since they significantly affect the reflected irradiance estimation (see below in details among others in connection with FIG. 16).

In an embodiment reflection parameter (which can be utilized in the calculation of indirect irradiance) of at least a part of the 3D elements of the 3D model may be determined (i.e. estimated, preferably based on recognizing a material type of the surface corresponding to the respective 3D element, viewing to the radiation position) by means of a target/material type learning algorithm applicable to an optical representation of the scene corresponding to the 3D model (wherein the correspondence between the optical representation and the respective 3D elements). This target/material type learning algorithm can be trained appropriately based on known material recognition training approaches.

One way to determine these parameters in the model is to use learning algorithms (e.g. neural networks) to distinguish different materials. These parameters can also be estimated by using an object detecting method combined with the obtained colour values (see above the determination of the reflection parameter). Object detection can be performed by learning the semantic interpretation of usual scenes (as will be detailed below; other words one type of learning is to recognize the objects or hidden areas, and calculating the irradiation plan for them). In both cases, the obtained parameters can be stored along with the corresponding elements of the 3D model. It might be rather useful to recognize different materials inside the room since the type of the material might affect the irradiation time required to properly disinfect that area.

Although an initial 3D model is required to perform the irradiance estimation and path optimization, it might be assumed that the environment changes over time. To take this factor into consideration the mobile robot should detect changes in the environment and modify the 3D model (and potentially the irradiance estimation and path) accordingly. On the one hand, the changes in the environment mean changes to the real irradiance values on surfaces and consequently recalculation might be required if the change is significant. On the other hand, the changes might mean that new obstacles appeared in the 3D map and some areas can no longer be approached by the mobile robot.

Consequently, while the robot performs the disinfection it may keep track of potential changes in the environment. If significant changes are detected, the robot may explore the whole environment again in order to find every meaningful difference from the original 3D map.

After that, the robot may suspend its operation and adapt the 3D model of the environment to these changes. The radiation plan then can be reoptimized considering that the robot might have already disinfected certain parts of the room. Once the reoptimization is completed, the robot can continue its operation.

Accordingly, in preferred cases:
any changes are preferably detected in the pre-defined or already scanned and mapped area, and making report (in Log) about the new obstacles;
the obstacles are recognized with reflection model as well;
irradiation plan is made by virtual ray-tracing for the new obstacles;
path is recalculated for an area with a recommended path and new detected obstacles to get optimal irradiation.

The SLAM algorithm that is preferably applied in the framework of the invention is able to reconstruct the environment in the form of an occupancy grid (3D elements: e.g. voxels plus occupancy values). However, other representations can be used too. For instance, a traditional point cloud (x, y, z coordinates) can also be extended with additional information (e.g. colour, uncertainty, reflection parameter) thus it can be used similarly to an occupancy grid. This representation can be created by many SLAM algorithms or e.g. by Structure from Motion methods using visual sensors, volumetric sensors, range sensors etc. For Structure from Motion methods, see e.g.:
COLMAP (Johannes Lutz Schönberger and Jan-Michael Frahm. Structure-from-motion revisited. In Conference on Computer Vision and Pattern Recognition (CVPR), 2016.)
OpenMVG (Pierre Moulon, Pascal Monasse, Romuald Perrot, and Renaud Marlet. Openmvg: Open multiple view geometry. In International Workshop on Reproducible Research in Pattern Recognition, pages 60-74. Springer, 2016.)
Theia (Christopher Sweeney, Tobias Hollerer, and Matthew Turk. Theia: A fast and scalable structure-from-motion library. In Proceedings of the 23rd ACM international conference on Multimedia, pages 693-696, 2015.)

Furthermore, the environment can be thought of as a set of objects which can be detected and recognized during the mapping process. In this case, the 3D model consists of a group of objects and their corresponding positions and orientations (one type of Learning is based on conventional vector-space based clustering for the objects).

In order to precisely compute irradiance values for the environment, its 3D model has to be discretized. Consequently, if the environment consists of a set of objects, these components also need to be discretized. Then the environment will be represented as a set of objects each of which is characterized by its position and orientation but also has a corresponding discretized 3D model which describes the shape of the object. This environment representation can be obtained by combining the 3D reconstruction method with an object detecting algorithm. The objects may be detected during the 3D mapping process or after it is completed. This way the concrete representation of the environment (objects) will not affect how it has to be reconstructed.

In the corresponding FIG. 2 environment reconstruction is illustrated in an embodiment, i.e. for an operational step of environment reconstruction S110.

According to the above description, in order to correctly estimate irradiance in the target environment (scene) and optimize the radiation positions of the light source, a sufficiently detailed 3D map (3D model) of that environment needs to be created. This task can be accomplished most easily by a mobile robot, but it has to be noted that the sensor configuration can be moved by humans and a prebuilt 3D model can also be used.

In the case of a mobile robot, it has to be equipped with the appropriate sensor configuration. A 3D model can be created using RGB cameras, depth cameras, LIDARs etc., but in order to distinguish different materials colour images are mandatory. In addition to the visual and/or volumetric sensors, position sensor information should also be obtained (e.g. GPS, IMU, odometry etc.) to reconstruct the environment more accurately. Accordingly, at the beginning of the environment reconstruction in an operational step S111, a mobile robot equipped with a sensor configuration that can be used for 3D reconstruction and localization is placed in the area of operation (i.e. into the environment, scene to be modelled).

As shown in FIG. 2, in this embodiment, the robot can be controlled three different ways during the exploration process. In an enclosed area the robot may navigate by itself if it is prepared to avoid obstacles (cf. operational step S112a, the mobile robot explores the area autonomously). Especially in a case, when the area is more or less static and open, the operator may design an exploration path for the robot along which it can move autonomously (cf. operational step S112b, the mobile robot moves along a predefined path). Lastly, the robot may be controlled and supervised by the operator during the exploration process (cf. operational step S112c, the mobile robot is controlled manually).

However, although in this section, the reconstruction is performed by a mobile robot according to each possibility of operational steps S112a, S112b, S112c, the 3D reconstruction can be performed in other ways (not using a robot, but another reconstructing apparatus). This is in line with that the radiation plan—being the result of the method according to the invention—may also not only performed by the help of a robot.

In either case a robot platform is advantageous since it can rigidly bind the sensors together. During the exploration process the robot records sensor information about the environment and its own positions (cf. operational step S113). Using this sensor information, a discretized real-scale 3D map can be created (cf. operational step S114). As mentioned above, this task can be performed online during the exploration by for instance a Simultaneous Localization and Mapping algorithm, but the 3D model can also be created after the operation by for instance a Structure from Motion pipeline.

The 3D model can be represented as a point cloud, a voxel grid or as the set of connected objects, etc. It is very important to build a 3D model with real scale since otherwise the distance from the light source that significantly affects irradiance estimation could not be measured.

Although optional, another important factor in the calculations is the set of local surface normal vectors which are used on the one hand to consider the angle of incidence for the incoming ray and on the other hand to compute the direction of reflected rays (cf. operational step S115, in order to allow for precise direct and indirect irradiance estimation, the local surface normal vector for each element in the 3D model is computed). The local surface normal vector for each 3D element can be estimated by for instance a function of the pcl library (R. B. Rusu and S. Cousins, “3D is here: Point Cloud Library (PCL)” in IEEE International Conference on Robotics and Automation (ICRA), Shanghai, China, May 9-13, 2011) and they are stored connected to the corresponding 3D elements.

In the case of the applied 3D model, although it consists of cubic volumes, these elements are considered as parts of the surfaces (i.e. a voxel/3D element/cell is a surface element, it is not subdivided into the surfaces of the cube). This simplification reduces the computational demand, while the irradiance results will be essentially accurate, since the 3D elements are relatively small. When surface normal vectors are computed, the centres of the elements are used to form surfaces to which normal vectors can be computed.

Using the colour images that were preferably captured during the exploration a neural network can be trained to distinguish different materials in the 3D model similarly to an object detecting neural network. Once the types of the different surfaces are known, their reflection parameters can be estimated and stored connected to the corresponding elements of the 3D model. This information is later used to estimate reflected (indirect) irradiance more precisely (cf. operational step S116, the images captured during the exploration process are used to distinguish different materials and the corresponding reflection parameters in the 3D model using a pretrained neural network).

In the following some exemplary details are given about the irradiance estimation.

In order to estimate irradiance in different positions of the robot, the currently affected voxels have to be selected in the 3D model. As a simplification—since these can be corrected easily in a way which is not in the scope of the invention—instead of real UV-C tubes, a simplified model has been applied in the experiments which assumes that the light source coincides with the camera centre and only radiate within the field of view of the device. An algorithm which can select the affected voxels has been created with this assumption (the reconstructed objects and the field of view parameters of the camera may be used as the input of the simulation software—the currently affected surface elements can be selected based on these parameters) and can be easily adjusted to different light source models.

If this method is applied, then the algorithm basically cuts out a subset of 3D elements from the whole 3D model which falls within the field of view of the light source and assumes that these elements are affected by the UV-C light. However, this algorithm does not consider that some 3D elements may actually be covered by closer 3D elements thus the irradiation simulation may be partly incorrect. To solve this problem, the methods based on raytracing or the comparison of global and local 3D models (e.g. point clouds) can be preferably used.

This method that is based on the field of the light source can be used to select currently visible parts of the 3D model (other approaches are preferred) when direct irradiance estimation is performed, however, for indirect irradiance estimation raytracing definitely have to be applied.

However, the method may take some voxels into consideration which are actually covered by closer objects, which might result in a partly incorrect simulation (see also below the description of FIGS. 3-4, the currently affected elements can be selected by matching the local measurements to the global map). When the 3D mapping of the environment is performed the global 3D model is composed of local measurements (e.g. point cloud) that correspond to the locations of the robot. For the elements of the 3D model, it may be stored that from which local measurements it was reconstructed, more precisely the corresponding robot positions can be stored. By storing this information for lots of robot positions, it can be used to estimate that which 3D elements are visible from certain radiation positions. This method may not be perfect since the robot positions stored during the mapping are not necessarily identical to the radiation positions. However, the problem of taking covered elements into consideration is solved, since those elements cannot be part of the corresponding local measurements if they are not visible.

In FIG. 3 a flowchart is shown which describes how a single disinfection plan which contains radiation positions and times is used along with the obtained 3D model to provide an irradiance evaluation method which is the basis for the optimization methods. Accordingly, an embodiment of irradiance evaluation is illustrated in FIG. 3, which is performed under operational step S120.

Accordingly, in FIG. 3 the procedure of the irradiance evaluation is illustrated in an embodiment showing how the irradiance is evaluated a radiation (disinfection) plan.

In operational step S121 the disinfection plan under consideration is divided into discrete radiation positions and the corresponding radiation times. According to the invention, this division is trivial, since the radiation (disinfection) plan is a priori built from pairs (each constituting a radiation data pair) of one or more radiation position and the corresponding one or more radiation time.

As shown in FIG. 3, there are two iteration loops within the irradiance evaluation. The first irradiation loop takes one-by-one the one or more radiation data pair. Within that, the second irradiation loop takes one-by-one the 3D elements (e.g. voxels) of the 3D model (see also below a possible simplification of this, where—in order to decrease computational demand—only every n^{th} 3D element is taken into account).

Accordingly, in operational step S121, a radiation (disinfection) plan (which can be a plan candidate coming from a radiation plan generator 130) is considered and in operational step S122, a radiation position and the corresponding radiation time, i.e. a radiation data pair of the radiation plan is selected (operational step S128 comes back to this point to start a further iteration cycle for the next radiation data pair, if there are still any radiation data pair available).

In operational step S123 a 3D element is selected in the 3D model. Considering also operational step S126, in the present embodiment we go through all element of the 3D model.

Within this inner operation loop starting with operational step S123, irradiance contributions are gained for each 3D element from direct irradiance and indirect irradiance. Accordingly, in operational step S124 direct irradiance is estimated for the given 3D element and after that in operational step S125 indirect irradiance is estimated for one or more other 3D elements by considering reflection (see FIG. 4 for details).

The followings are noted for the interpretation of operational steps S124 and S125 in FIG. 3.
Since these steps are within two iteration loops the direct irradiance is checked for a given 3D element for irradiance coming from a given radiation position for a given radiation time. Accordingly, it is clear for a given 3D element what contribution is to be taken into account.
For the indirect radiation it is analysed whether the radiation reaching the given 3D element—which is currently selected—is reflected or not (this is indirect irradiance) to another 3D element. It can be reflected to one or more other 3D element. Going through the 3D elements, for the given 3D element, it is collected that to which other 3D elements contribution is given via the indirect radiation, i.e. not only the given 3D element can receive a contribution in an iteration cycle, but—via indirect irradiance—other 3D elements also (in other words, we go through the 3D elements one-by-one but also those 3D elements can receive contribution which are not currently selected; i.e. the 3D elements can gain contribution in every cycle of the iteration loop).
The factors that are considered for direct and indirect irradiance estimation are detailed in connection with FIGS. 4A-4B and 16 below.

In operational step S126 it is investigated whether all 3D elements (of the 3D model) have been selected or not. If not, we go further to a next 3D element. However, if yes, we jump to operational step S127 where an irradiance distribution map is generated (created) by storing the computed irradiance values connected to the 3D elements. This means that the irradiance distribution for a given radiation data pair is stored for further use (see also operational step S129).

After storing this intermediate irradiance distribution map for a given radiation data pair, it is checked in operational step S128 whether all radiation positions (of the current radiation/disinfection plan) have been selected or not. If not, we go back to operational step S122 and select a further radiation data pair. If yes, i.e. all radiation positions of the investigated radiation (disinfection) plan have already been selected, we proceed to operational step S129, where the individual (intermediate) irradiance distribution maps (if there are more, since there are more radiation data pair) corresponding to each of the radiation positions are fused to determine the final quality of the current disinfection plan.

The followings are noted in connection with operational step S129 in FIG. 3.
Via the fuse it is summarized for each 3D element of the 3D model that what irradiation contribution it receives from the radiation coming from the one or more radiation positions. Accordingly, information can be obtained for each 3D element for the total received irradiation.
Furthermore, considering a level to be reached by the irradiation, it can be determined for each of the 3D elements whether it is irradiated sufficiently or not. Therefore, those 3D elements which are sufficiently irradiated can be counted. By dividing the number of the sufficiently irradiated 3D elements by the total number of 3D elements, the irradiance ratio estimation can be obtained for the currently investigated radiation (disinfection) plan.

It is noted as a summary that, preferably, an irradiation map is created for all radiation position—radiation time pair, i.e. it is stored how much irradiance reach a 3D element from a radiation position for the duration of the corresponding radiation time. However, all of the 3D elements may receive irradiation from other radiation positions of a radiation plan candidate (if any), so this effect on them is also to be collected in the 3D elements in order to decide about whether it is irradiated sufficiently (i.e. give a contribution to the irradiance ration estimation or not).

In connection with this it is noted that in FIG. 1B, it is shown that optimally irradiated maps 137 (or a single map, but for dynamical visualisation more maps are needed according to the stages) may be generated based on the data of irradiance estimation module 120′ (according to that the summed-up irradiance data are available at the end of the iteration).

In a further embodiment (note that in the embodiment of FIG. 3, all 3D elements are investigated), in order to accelerate the irradiance estimation during the optimization process, preferably, a subset of the 3D elements can be used for the evaluation instead of computing the values for all elements. In most cases it may be enough to compute the values for each n^{th} element (where n is determined empirically) but only if this simplification does not affect the order of possible solutions regarding their fitness score, or any other score that reflects how good a solution is.

Although the irradiance estimation is not performed for them, the algorithm iterates every 3D element. If the current element is the n^{th} in the iteration, then the irradiance value is computed for it. Otherwise, the irradiance is not increased. If irradiance is computed for an element, then the indirect irradiance estimation should also be performed. By considering reflected rays, it may happen that for certain voxels only indirect irradiance values are computed.

The ratio of sufficiently well disinfected elements is computed the same way (irradiance ratio estimation) as if the irradiance was computed for every element, thus all irradiance increment will be taken into consideration. Although, the ratios will be lower compared to the case when irradiance values are computed for every voxel, the order of radiation plan candidates should be the same. The ratio of the fitness scores obtained through considering only every nth element and the original fitness scores is approximately 1/n. The required irradiance level is adjusted similarly thus the optimization can provide the optimal disinfection plan.

For instance, if the original values of the fitness scores of a first and second individuals and the limit were 0.7, 0.6 and 0.75 respectively then the corresponding values considering the above described simplification for the case when n is equal to 10 were approximately 0.07, 0.06 and 0.075 respectively. Since the order of the two individuals regarding their fitness scores did not change, neither their relation to the limit, the optimization can be properly executed. The value of n may be in the range between 2 and 20 (preferably 2 and 15). Note that the algorithm may still work properly for certain cases for n values higher than 20 (e.g 50) but in these cases it has to be checked that the order of individuals regarding their fitness scores is not changed by applying this simplification.

Since the time of adding zero irradiance to an element is negligible compared to performing multiple raycasting, the resulting computation time will be approximately 1/n times the original computation time.

Considering this, it may be enough to examine e.g. every tenth element in the 3D model which would significantly reduce the computation times, while the order of radiation plan candidates will presumably not change. In contrast, by using an order of magnitude higher n not enough elements would be considered to preserve the correct order of radiation plans.

It has to be noted that this simplification could only be used during the optimization process. When the final results are computed for the radiation plan or the training dataset for the convolutional neural network is obtained, the irradiance values may be computed optionally for all the elements in the 3D model (but the 3D model and a corresponding radiation plan computed by the method according to the invention based on evolutionary algorithm, incremental path generation or reinforcement learning are the minimum input requirement for training in the embodiment based on deep learning).

Accordingly, in an embodiment corresponding to FIG. 3 (for the direct and indirect irradiation, see the details in connection with FIGS. 4A and 4B), at least a part of the plurality of 3D elements of the 3D model constitutes investigation 3D elements having a number of investigation 3D elements (as given above, group of the investigation 3D elements may comprise all 3D elements or at least a part of them: every n^{th} 3D element).

In this embodiment, in course of generating an irradiance ratio estimation for a plan candidate in the irradiation evaluation step,
a respective one or more irradiance map of the investigation 3D elements is generated by estimating an irradiance (see below, this can be a direct irradiance, but optionally additionally indirect irradiance; here the irradiance is estimated without further constraints) for each of the investigation 3D elements for each of the one or more radiation position with the corresponding radiation time (i.e. an irradiance map is generated for all of the radiation positions),
a fused irradiance map of the investigation 3D elements is generated by summing up the one or more irradiance map of each of the one or more radiation positions (i.e. which corresponds to the one or more respective radiation position) for all of the one or more radiation positions (i.e. all irradiance maps give their own contribution to the fused irradiance map),
a number of the investigation 3D elements with irradiance exceeding a predetermined irradiance threshold from all of investigation 3D elements are counted as a number of sufficiently irradiated 3D elements (i.e. it is counted how many of the investigation 3D elements have irradiance higher than the predetermined irradiance threshold), and
the irradiance ratio estimation is obtained as a ratio of the number of sufficiently irradiated 3D elements and the number of investigation 3D elements.

The direct and indirect irradiance values are estimated according to the same Irradiance Estimation Formula (see below) which contains the parameters described by FIG. 4A (for direct irradiance) and FIG. 4B (for indirect irradiance).

Referring to FIG. 4A the computation process of direct irradiance values in an embodiment is as follows (illustrated by the description of direct irradiance estimation operational step S150a, this can be applied in the framework of FIG. 3 substituted into operational step S124). Accordingly, the sequence of the steps is started by an operational step S123a, wherein a 3D element is selected in the 3D model.

First the distance between the light source and the irradiated surface is computed. This is in connection with operational step S151a in FIG. 4A, wherein distance is measured between the light source and the direct target surface element (i.e. generally between the radiation position and the 3D element under investigation; see above that the surface of a 3D element is treated in this way, the 3D element can have a colour also). This follows from that after a certain distance (the predefined distance limit is e.g. set to 10 m in the experiments, but it depends on the power of the lamp; generally it may be e.g. between 5 and 25 m, in particular 8 and 15 m, and also it can be an input how low value is negligible) the irradiance values are negligibly small thus the computation for them is not worth the time. Consequently, further calculations are carried out only for those surface elements that are closer than the predefined limit. Thus, in operational step S152a it is investigated whether the distance is greater than a predefined limit.

If the distance is found to be greater than the sequence of steps is terminated for the given 3D element (it is too far from the radiation position) and according to the “Yes” branch, operational step S123b follows, wherein the direct irradiance increment is set to zero for the given 3D element (this happens also for certain output for another decisions, see FIG. 4A).

Out of the other three parameters that depend on the structure of the environment either can take the value of zero due to different reasons. Such a reason is investigated if the distance is below the predefined limit. Then, the sequence of steps continues with operational step S153a, wherein angle of incidence is computed for the ray coming from the light source in the currently investigated 3D element.

The angle of incidence may be zero if the ray is parallel to the surface or if the local surface normal vector could not be computed for that 3D element. This latter can be handled in this step also.

Accordingly, in operational step S154a it is investigated whether the angle of incidence is equal to zero or not. If it is equal, then operational step S123b follows and the direct irradiance increment is set to zero for the given 3D element (although it was close enough to the radiation position).

If the angle of incidence is different from zero then in operational step S155a the pose of the light source relative to the direct target surface element is considered through the characteristics of the light source. Accordingly in operational step S156a it is investigated whether the light source characteristics parameter is negligibly small or not. If yes, the procedure is terminated by operational step S123b. The parameter that encodes the light source characteristics usually takes a positive value but for instance directly above a cylindrical light source the computed values may be negligibly small.

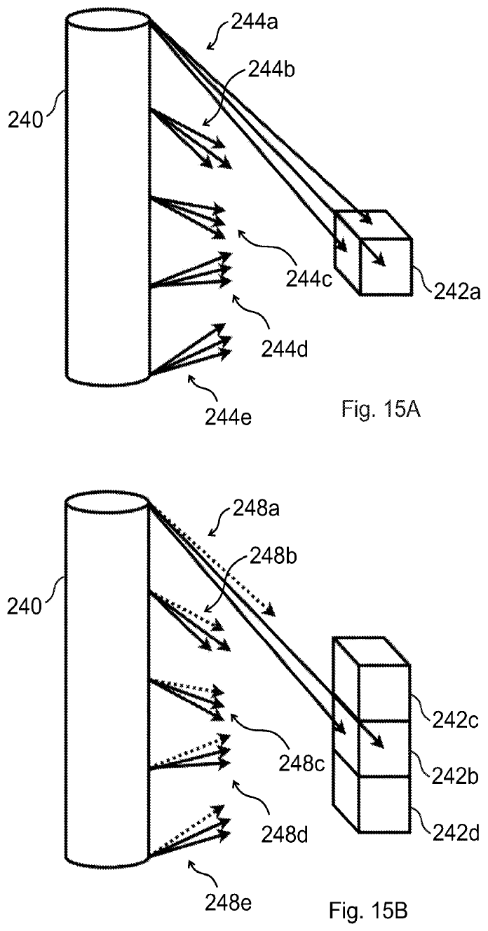

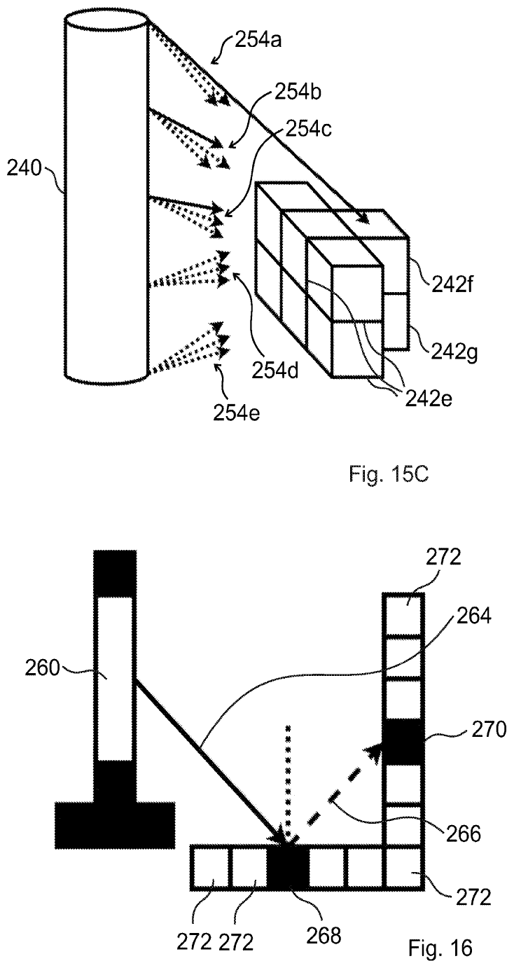

Lastly, the visibility parameter is computed in operational step S157a (see FIGS. 15A-15C for the computation of the visibility parameter in an embodiment). The visibility parameter may be zero if a surface element cannot be seen from the viewpoint(s) of the light source. Accordingly, it is investigated in S158a whether the given 3D element is visible from the viewpoint of the light source. If no, then the procedure terminates by operational step S123b: there is no visibility, therefore the direct irradiance increment is set to zero for the given 3D element.

Although the visibility parameter has the biggest chance of taking a zero value, it is also the one that takes the most time to compute due to the lots of ray casting.

Consequently, these three parameters are computed in the following order: angle of incidence, light source characteristics parameter, visibility parameter. The time of irradiation and the parameter which is proportional to the power of the light source cannot take zero values and do not depend on the structure of the environment.

The final irradiance value for a given surface element is only computed according to the Irradiance Estimation Formula if all four uncertain parameters take positive values. It is performed according to the “Yes” branch of operational step S158a, in which case in operational step S159a the direct irradiance increment is estimated based on the aforementioned parameters according to the Irradiance Estimation Formula where the power of the light source and the time of irradiation are also considered.

Accordingly, in an embodiment a direct irradiance is taken into account in the irradiation evaluation step for generating the respective irradiance ratio estimation for each of the one or more plan candidate. In this embodiment it is specified that the direct irradiance is utilized in the calculations, although other type of irradiance—like indirect irradiance—is not excluded.

In an embodiment using the above specifying, the followings are further defined according to the details specified in FIG. 4A. A direct irradiance increment is calculated in course of estimating (for obtaining the irradiance ratio estimation finally) the direct irradiance of each investigation 3D element for each radiation position by investigating
whether a first distance between the investigation 3D element and the radiation position is greater than a first predetermined distance limit (cf. operational step S152a of 4A),
   in case of being greater, the direct irradiance increment for the investigation 3D element is set to zero (cf. operational step S123b of FIG. 4A which is a common output for several investigations, i.e. decision points),
   in case of not being greater, by investigating (cf. operational step S154a of FIG. 4A) whether an angle of incidence of the direct irradiance is equal to zero,
   in case of being equal to zero, the direct irradiance increment for the investigation 3D element is set to zero (here, also operational step S123b comes up),
   in case of not being equal to zero, by investigating (cf. operational step S156a of FIG. 4A) whether a radiation source characteristics parameter (cf. I(α(x)) in connection with FIG. 18) for the investigation 3D element has a value below a predetermined characteristics parameter limit,
      in case of being below, the direct irradiance increment for the investigation 3D element is set to zero (here, also operational step S123b comes up),
      in case of not being below, by investigating (cf. operational step S158a of FIG. 4A) whether the investigation 3D element is visible from the radiation position,
         in case of not being visible, the direct irradiance increment for the investigation 3D element is set to zero (here, also operational step S123b comes up),
         in case of being visible, the direct irradiance increment of the investigation 3D element is estimated based on an irradiance estimation formula (cf. operational step S159a of FIG. 4A; for the evaluation of the irradiance estimation formula, see the comments above given in connection operational step S159a).

FIGS. 15A-C describe the concept of the visibility parameter in the Irradiance Estimation Formula. From several source points on the surface of the lamp three-three rays are cast which target the three closest surfaces of each target 3D element. In FIG. 15A, only three rays of bundle 244a are shown that directly reach the target 3D element, however each ray that is marked solid (of every other bundle 244b-244c) technically reaches the target (they are shorter to make the image easier to observe).

When the visibility parameter is computed it is only important that one of the three rays reaches the target and if so, the target is marked visible from the given source point. Then the visibility parameter is computed by dividing the number of those source points from which the target 3D element was visible by the total number of source points (a single, stand-alone 3D element 242a—in the figure, a voxel—is considered). In FIG. 15A, the visibility factor of the target 3D element (considering the current lamp position) will be 5/5=1. This shows that it is independent from the number of rays casting from the lamp.

In FIG. 15B those rays that were cast, but could not reach the target due to an occlusion are marked by dotted arrows (two solid rays of bundle 248a are shown as reaching the sides of a 3D element 242b, the dotted ray does not reach it; all the other bundles 248b-248e will have two solid rays and a dotted one, as it can be easily understood). In FIG. 15B, where the 3D element 242b arranged between two other 3D elements 242c, 242d—one on the top, and one below—the visibility factor of the target 3D element (considering the current lamp position) will be 5/5=1. Note that although the target is covered from above and below, the visibility factor is 1 since other two surfaces can be reached from all of the source points on the lamp.

In FIG. 15C a 3D element 242f (voxel) is the target 3D element. The 3D element 242f is arranged centralized behind a 2 (height)×3 (width) wall of 3D elements 242e (the top of a 3D element 242f is at the same level as the wall, and a 3D element 242g is arranged below the 3D element 242f). The visibility factor v of the target 3D element 242f (considering the current lamp position) will be 3 (the number of those source points from which the target 3D element 242f is visible)/5 (total number of source points)=0.6 (the top of the 3D element 242f is visible from the top three source points: these bundles 254a-254c have a solid ray; and the 3D element 242f is not visible from the bottom two source points, bundles 254d-254e have only dotted ray). Note that in this case the target is completely covered from the bottom two viewpoints on the lamp thus the visibility factor cannot be maximal.

In the following the computation of for indirect irradiance estimation is illustrated. The computation process is similar for indirect irradiance estimation (realized in operational step S150b in an embodiment; this step can be substituted instead of operational step S125 in FIG. 3) illustrated in FIG. 4B, considering the differences described herebelow.

The steps for giving the indirect irradiance estimation for the selected 3D element. As touched above, it is investigated for the indirect irradiance case whether the selected 3D element gives contribution to one or more further 3D elements via reflection. Naturally, by the final summation of this contribution it is important to check whether the selected 3D element itself received irradiance or not, since it can give contribution via reflectance if it is irradiated. In case the reflectance is taken into account then the value of irradiance of a direct target element may be adjusted according to the reflectance.

For a given (selected) 3D element, as a first operational step S151b the distance is measured between the direct target surface element (corresponding to the selected 3D element) and the indirect target surface element. If the distance is too large (in the reflecting direction, there is only a 3D element too far), then the indirect irradiance gives no contribution (see also below for specifying how the levels of the investigation are built up).

If the distance is under the predefined limit, then in operational step S153b the angle of incidence is computed for the reflected ray (in respect of the 3D element onto where it is directed). If this angle is zero then the indirect irradiance increment is set to zero for that 3D element onto which the reflected ray is directed.

If the angle of incidence is not zero, then the procedure is continued. In operational step S155b, since the reflecting surface is not a real light source, its reflection parameter is used in the irradiance estimation formula (instead the characteristics of the light source). Thus, if the reflection parameter is too small, then the procedure is terminated.

If this is not the case, in operational step S157b the visibility parameter is computed according to whether the reflected ray reached the target. Note that the visibility parameter in the case of reflected irradiance estimation is computed directly when the reflected ray casting is performed. Since no cylindrical light source is present in the context, only one source point is selected. Furthermore, as not a specific surface element is targeted, only the direction is known, it is enough to cast only one ray from that one source point. Obviously, if it is assumed that the reflection is not perfect and the reflected ray is scattered then it can be represented by multiple beams inside a cone. However, in the simplest case, when only one reflected ray is cast, the visibility parameter will be either one or zero according to whether it reached another surface element inside the 3D model or not respectively.

Accordingly, if the indirect target surface element (i.e. the 3D element) is visible from the selected 3D element, then in operational step S159b indirect irradiance is estimated based on the aforementioned parameters according to the irradiance estimation formula where the direct irradiance of the source selected 3D element and the time of irradiation thereof are also considered as referred above.

Accordingly, in an embodiment—being built on the embodiment in which the direct irradiance is taken into account—an indirect irradiance is further taken into account in the irradiation evaluation step for generating the respective irradiance ratio estimation for each of the one or more plan candidate.

In a related embodiment, it is further specified an indirect irradiance increment is calculated in course of estimating the indirect irradiance of each investigation 3D element constituting a direct target 3D element (cf. a direct target 3D element 268 of FIG. 16) for each radiation position by modelling at least one reflected ray (maybe more, but typically one, also cf. reflected ray 266 of FIG. 16), choosing respective at least one indirect target 3D element (cf. indirect target 3D element 270 of FIG. 16) based on an incoming ray (cf. incoming ray 264 of FIG. 16) falling onto the direct target 3D element from the radiation position and investigating for each of the at least one indirect target 3D element
whether a second distance between the direct target 3D element and the indirect target 3D element is greater than a second predetermined distance limit (this is the investigation performed after operational step S151b),
   in case of being greater, the indirect irradiance increment for the indirect target 3D element is set to zero (this happens similarly—also at other outcomes below—as in the case of FIG. 4A),
   in case of not being greater, by investigating whether an angle of incidence of the reflected ray onto the indirect target 3D element is equal to zero (this investigation is performed after operational step S153b, wherein the angle of incidence is calculated for this case),
   in case of being equal to zero, the indirect irradiance increment for the indirect target 3D element is set to zero,
   in case of not being equal to zero, by investigating whether a reflection parameter of the direct target 3D element has a value below a predetermined reflection parameter limit (this is done after operational step s155b),
      in case of being below, the indirect irradiance increment for the indirect target 3D element is set to zero,
      in case of not being below, by investigating whether the indirect target 3D element is visible from the direct target 3D element (this is investigated after operational step S157b),
         in case of not being visible, the indirect irradiance increment for the indirect target 3D element is set to zero,
         in case of being visible, the indirect irradiance increment of the indirect target 3D element is estimated based on an irradiance estimation formula (cf. operational step S159b).

Except for the visibility parameter, the computational order of the other factors may be changed for both direct and indirect irradiance estimation.

First and second predetermined distance limit (there is an exemplary 10 m is given for the first predetermined distance limit elsewhere; the second predetermined distance limit can be lower than the first one, since the reflected light becomes negligible on shorter distance), as well as predetermined characteristics parameter limit and predetermined reflection parameter limit are given as inputs to the respective embodiment of the method according to the invention.

In FIG. 16, it is illustrated that not only direct but indirect irradiance is preferably computed as well. Let us assume that the black square on the bottom is voxel1 (generally, direct target 3D element 268) and the black square on the right is voxel2 (generally, indirect target 3D element 270), wherein the direct target 3D element 268 and the indirect target 3D element 270 are arranged among 3D elements 272, while a lamp 260 on the left is the light source. A solid arrow illustrates the incoming ray 264 (beam; direct irradiance), a dashed arrow is the reflected ray 266 (beam; indirect irradiance) and the dotted line is the surface normal vector.

The irradiance value of voxel1 is computed according to the Irradiance Estimation Formula. The indirect irradiance is computed using the same expression with a few minor assumptions. First, the voxel1 becomes the light source and voxel2 becomes the target voxel. Second, the parameter in the Irradiance Estimation Formula that is proportional to the power of the lamp will be equal to the computed direct irradiance of the voxel1. Finally, since no real lamp is present in this context (i.e. in the case of indirect irradiance), the lamp characteristics parameter will be equal to a reflection parameter. This reflection parameter describes how well the given surface can reflect the incoming ray (perfect reflection means a perfect 1 value for this parameter).

As mentioned above, the reflection parameters for each 3D element can be estimated during the 3D reconstruction process by training a neural network that can recognize different materials and thus these values can be stored connected to each 3D element. Lastly, it can be easily seen that multiple reflections can be performed with similar assumptions.

In order to compute irradiance distribution in a location around, first those surface elements had to be selected which were affected by the UV-C light (it can be also said that ray-tracing methodology to calculate the irradiation in 3D for an UV-C lighting robot along a pre-defined or automatically driven path). As it was mentioned above, this challenge was addressed by performing raycasting, and only those voxels were taken into account which were closer to the lamp than a predefined maximum distance (10 m in the experiments, see also the first predetermined distance limit, which is equivalent to this). To deal with the cells that were covered due to the imperfection of the map representation, multiple rays were cast in the direction of the target voxel, this is illustrated in FIGS. 15A-15C.

The reconstructed objects and the field of view parameters of the camera may be used as the input of the simulation software. The irradiance estimation is preferably executed cyclically based on the measured distance from the camera centre, the angle of incidence and time.

In the following the Irradiance Estimation Formula is introduced by the help of interpretation of the different factors.

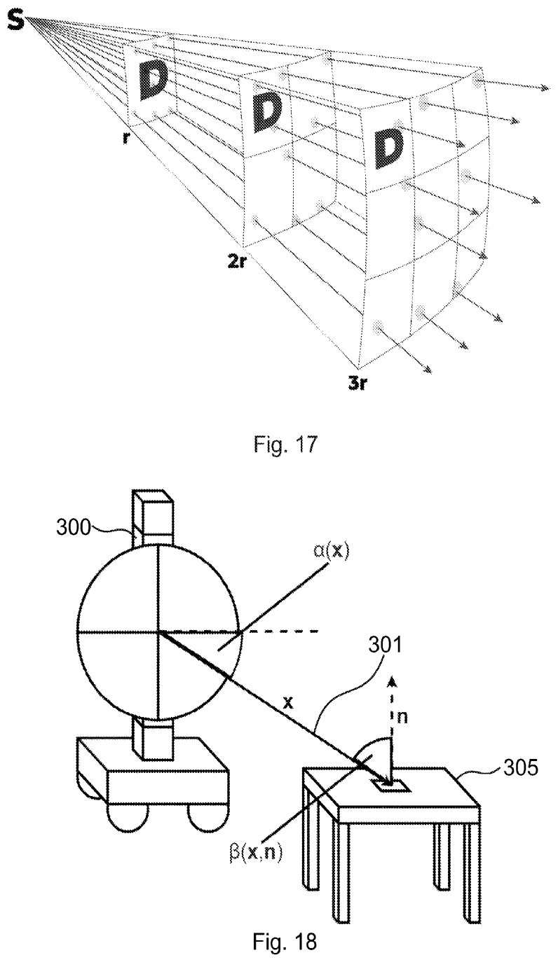

The distance between the lamp (generally, radiation position) and the irradiated surface element (generally, 3D element) is taken into consideration according to the inverse square law. As it is illustrated in FIG. 17, the further the target from the light source, the larger the surface over which the constant radiant power is distributed.

Therefore, the estimated irradiance (in other words the delivered power) had to be inversely proportional to the square of the distance between the voxel and the lamp (generally, radiation position), since the lamp was considered as a point light source and the surface area of a sphere is proportional to the square of its radius.

However, the measured characteristics of the lamp is only valid if the distance r is greater than a lower limit (e.g. eight times the height of the lamp, or other lower limit may be chosen, e.g. between 1-8 m). For those surface elements that were closer to the light source, the distance was set to be equal to this limit, thus the irradiance was underestimated. The distance r is computed using the voxel and camera locations in the global 3D map and its impact on irradiance is described by

$$I ( r ) = \frac{k}{{r}^{2}}$$

where k is a constant.

FIG. 18 shows the visualization of two angle parameters, i.e. the angle of a beam 301 (ray) to the horizontal plane α(x) (see also below) and the angle of incidence β(x,n)) by the help of a light source 300 and a table 305. The I(α(x)) parameter describes the characteristics of the light source, i.e. the distribution of irradiance on a spherical surface around it. The vector pointing from the centre of the light source to the radiated surface and the local surface normal vector are denoted by x and n respectively. The I(α(x)) parameter is connected to the lamp and describes its characteristics which usually needs to be measured. The β(x,n) parameter however depends on the 3D model and the relative placement of the lamp. The local surface normal vectors are preferably computed once the 3D reconstruction is complete and are stored connected to the 3D elements.

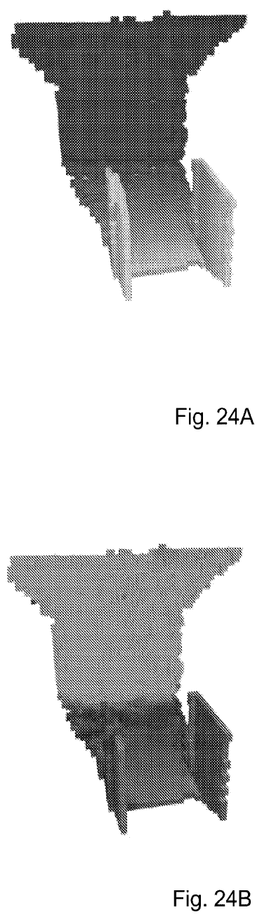

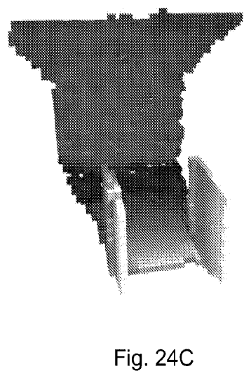

The irradiance is maximal, if the beam 301 is perpendicular to the surface. However, the greater the angle of incidence β(x,n) (i.e. the angle between x and the local surface normal vector n, the greater the area on which the radiant power is distributed and consequently the lower the irradiance, which is proportional to the cosine of β(x,n). In order to determine the angle of incidence, the surface normal vectors are computed using the normal estimation method provided by the pcl library (R. B. Rusu and S.Cousins, “3D is here: Point Cloud Library (PCL)” in IEEE International Conference on Robotics and Automation (ICRA), Shanghai, China, May 9-13, 2011). The angle of incidence for a specific surface element (of the table 305) is illustrated in FIG. 18. As it was mentioned above, multiple rays were cast in order to decide whether a voxel is radiated or not, however, both x and β(x,n) were computed based on a single vector pointing from the centre of the light source to the centre of the voxel. The dependency on the angle of incidence β (can also be denoted as θ) is shown by

$$I ( β ) = {I}_{dir} \cdot cos ( β )$$

where I_{dir} denotes a beam of irradiance perpendicular to the surface (i.e. it corresponds to the maximum/direct value of the irradiation, if only this component is considered separately). The effect of these factors is illustrated in FIGS. 20B-20E as well as FIGS. 24A-24C.

To summarize, the radiant exposure I (with upper case ‘I’ like ‘irradiance’; i.e. the irradiance of a surface integrated over time of irradiation; also referred to as ‘irradiance value’ or ‘irradiation value’ in this document) for each voxel was computed based on the lamp characteristics parameter I(α(x)) (with lower case ‘L’ for a lamp( ) function), the visibility factor v, the distance r(x) between the centre of the light source and the radiated surface, and also the angle of incidence β(x,n), according to the Irradiance Estimation Formula:

$$I ( l , v , β , r , t ) = \frac{l ( α ( x ) ) \cdot v \cdot cos ( β ( x , n ) ) \cdot t}{{r}^{2} ( x )} \cdot {Φ}_{ref} ,$$

wherein φ_{ref} denotes the radiant power transmitted to a perfectly visible voxel, 1 m from the centre of the lamp, for which the corresponding beam is parallel to the horizontal plane and perpendicular to the radiated surface. Since the efficiency of disinfection highly depends on the duration of the irradiation process, a time parameter t was also considered. Although, φ_{ref} was only used as a parameter, if its absolute value was measured, the created algorithm could be easily modified accordingly and real irradiance values could be obtained.

Since it is out of the scope of this description to perform disinfection in a real-world environment, it was sufficient to define φ_{ref}, t and the irradiance limit Iₗᵢₘᵢₜ, that corresponds to the proper disinfection level, as parameters. In the simulations, the unique key of each voxel was used to store its irradiance value. If the lamp was placed in more than one position to radiate, then the calculations were carried out separately resulting in multiple voxel grids. Once irradiance estimation has been completed, the 3D models were fused and the results were summarized (cf. FIG. 3), then the colour of each voxel was set according to the ratio of its irradiance value and Iₗᵢₘᵢₜ.

As a summary in connection with the various limits it is given that in an embodiment (bringing two sub options) in the radiation plan choosing step a radiation plan sufficiency criterion is applied being based on
a radiation time limit, whereby a summation of the one or more radiation time of the plan candidate is intended to be equal to or below the radiation time limit, or
an irradiance limit, whereby the irradiance ratio estimation of the plan candidate is intended to be equal to or above the irradiance limit.

In connection with the radiation plan choosing step, the followings are noted. In every embodiment of the invention, the (final) radiation plan is to be chosen (selected) in some way. Sometimes, it is chosen in dependence from various limits (e.g. the above radiation time limit and irradiance limit) which are checked time to time. This choice is preferably taken in the steps of an iteration. As it is clear from other embodiments the choice can be taken independently from limits (e.g. by reaching a predetermined maximum iteration number) or selected from the available plan candidates (such as in reinforcement learning based embodiment). Thus, these choices are all meant under the scope of radiation plan choosing step.

The optimization performed by means of the radiation plan generator module essentially can be based on two bases. In a first option for the cost is tried to be minimalized (an irradiance limit is to be reached within as low radiation time as possible; this is a minimum type limit, it is intended to be above the irradiance limit) or in a second option for base the quality is to be maximized (considering a radiation time limit, as high irradiance is reached as possible; this is a maximum type limit, it is intended to be below the radiation time limit).

If the reaching of the radiation time limit is expected from above, then, when reached by—limit breaking—one or more plan candidates (it is possible that there is one, but there can be more as well; it is intended that the limit is broken from above, so we have a low enough summed up radiation time for all radiation positions) then that plan candidate is selected as the (final) radiation plan which has the highest irradiance ratio estimation from—limit breaking—one or more plan candidates.

If the irradiance limit is expected to be reached, then when reached there will be—limit reaching—one or more plan candidates as well, but here that plan candidate will be selected which have the lowest total radiation time.

However, when the iteration is terminated before the radiation time limit or the irradiance limit is reached. In these cases, a further parameter is preferably applied for selecting the (final) radiation plan. In such a case that plan candidate is selected as the radiation plan which has the highest efficiency proportion of the irradiance ratio estimation and the total radiation time.

This efficiency proportion is a kind of efficiency parameter, see in the example below. For the application of the above principles, see the description of the applicable conditions and their fulfilment below.

Naturally, if there is only one limit reaching plan candidate then it will be selected, since evidently it will have the best score (selected from one score): irradiance estimation, total irradiation time or proportion.

Illustrating the above cases by an easy example, there are three solutions (all of them fit to the limits specified below):
a first solution with 0.86 irradiance ratio estimation and 5 minutes total radiation time (value of efficiency proportion 0.172 [1/min]),
a second solution with 0.83 irradiance ratio estimation and 3 minutes total radiation time (value of efficiency proportion 0.276 [1/min]), and
a third solution with 0.80 irradiance ratio estimation and 2.9 minutes total radiation time (value of efficiency proportion 0.275 [1/min]).

In case when the radiation time limit is applied in the radiation plan sufficiency criterion and value of 5 minutes is set (this value cannot be exceeded, but the parameter may be equal to this), then the first solution will be selected (since it has the highest irradiance ratio estimation from the solutions).

In case when irradiance limit is applied in the radiation plan sufficiency criterion a value of 0.8 is set (the irradiance ratio estimation has to reach this), then the third solution will be selected.

However, in case when the efficiency proportion is to be applied for the selection, then the second solution will be selected, since it has the highest value (thus, it has highest efficiency).

The sum of radiation times that correspond to the radiation positions can be normalised to be equal to a predefined maximum duration limit. In this case the ratio of radiation times would not change only their magnitude. If the radiation times are adjusted this way, the length of radiation plans (i.e. the number of radiation positions) would not be limited in theory. However, the algorithm preferably handles this problem by computing how increasing the number of radiation positions improves the fitness score of the best individual. For instance, if n is the current number of radiation positions, then the algorithm will compare the best solution of length n with the best solution of length n+1. If a sufficiently good radiation plan could not be obtained but the ratio of the fitness score of the best radiation plan of length n+1 and the fitness score of the best radiation plan of length n is under a predefined limit, the optimization terminates and the best available radiation plan is selected.

In a case when the efficiency proportion may be used, preferably, the best radiation plan can be defined in two ways.

On the one hand it can be checked which solution was closest to the predefined first and second optimization criteria. For instance, the first criterion can be to finish the disinfection under 20 minutes and the second to reach a 0.9 irradiance level. By normalization of radiation times, the first criterion can definitely be fulfilled and then from the appropriate solutions, the one with highest irradiance level will be selected. If the order of the two criteria is switched, then it is the most important aspect to be as close to the required irradiance level as possible and if multiple solutions provide the same irradiance limit (or the difference between them is under a predefined threshold) then the solution with the lowest duration will be selected.

On the other hand, the best available radiation plan can be defined so that it is the most efficient. The efficiency parameter (efficiency proportion) of a radiation plan candidate can be defined as the ratio of the obtained irradiance level and the corresponding radiation time. In this case, the radiation plan candidates can be easily sorted an the one with highest efficiency is selected. Note that these two selection methods for the best available solution can be combined as well by forming two new criteria. If there are multiple solutions that are closer to the required irradiance level and radiation times than predefined thresholds then the solution in the set with the highest efficiency can be selected. Similarly, if there are multiple solutions with very similar efficiency (an efficiency range with the highest efficiency being its maximum can be defined), the one which is closest to the required irradiance limit and radiation time can be selected.

It is noted in connection with the above, that if it is applied as a limit
beside the radiation time limit a first type auxiliary irradiance limit (e.g. 0.2) may be set defining a minimum irradiance value which have to be reached by the candidate even if the radiation time limit is sufficient;
beside the irradiance limit a first type auxiliary radiation time limit (e.g. 15 minutes) may be set defining a maximum radiation time value cannot be exceeded by the candidate even if the irradiance limit is sufficient.

The first type auxiliary limits are applicable in the iteration to be fulfilled at the same time as the base limit (radiation time limit or irradiance limit) and by the application of them it can be filtered out to have a sufficiently short time solution with too bad irradiance (in case of radiation time limit) or a too long solution with sufficiently high irradiance (in case of irradiance limit).

A second type auxiliary criteria can be applied if
the radiation time limit is approached from below then a proximity radiation time range can be defined which is characterized by its upper limit which is equal to the original/first radiation time limit and the size of the range (e.g. 0.5 minutes or maximum 1/10 of the value of the radiation time limit);
the irradiance limit is approached from above then a proximity irradiance range can be defined which is characterized by its lower limit which is equal to the original/first irradiance limit and the size of the range (e.g. 0.05 or between 1/10 and 1/20 of the irradiance limit).

It is thus preferably expected for obtaining a sufficiently good plan candidate to not only be below the radiation time limit or above the irradiance limit but being close enough to them (that is why it can be called proximity range).

The above auxiliary criteria are used in order to make use of the time available to obtain better disinfection results or not to reduce the irradiance ratio estimation necessarily to the limit in favour of saving time. The original/first radiation plan sufficiency criteria can be extended by the first and/or the second type auxiliary criteria.

All of the limits mentioned hereabove (and throughout the description) are predefined limits being inputs for the embodiment of the method. These can be selected according to a specific scene based on several factors (how much irradiation is expectable, how much time can be spent with radiation, etc.).

By leveraging the previously computed local surface normal vectors, the reflected UV-C rays can be also taken into consideration. Indirect irradiance is computed basically the same way as direct irradiance (see description of FIG. 16 above). When direct irradiance is computed, the algorithm iterates all elements in the 3D model (see S123 of FIG. 3) since the light source radiates in all directions. In contrast, for indirect irradiation it is assumed that the light source mainly radiates in one direction (or in a cone) or in other words the targeted surface mainly reflects in one direction. This direction can be computed by the traditional reflection law. When this main direction is computed, a ray can be cast from a reflecting (source) voxel (direct target 3D element 268 in FIG. 16) and an indirect target voxel (indirect target 3D element 270 in FIG. 16) can be selected for which the indirect irradiance value can be estimated.

The computation of indirect irradiance is performed by the Irradiance Estimation Formula just like the direct, with some modifications. The parameter I_ref (which is proportional to the power of the light source in direct irradiation) will be proportional to the computed direct irradiance for voxel1. I(α(x)) (light source characteristics) is not used in its original form since no real light source is present. Consequently, I(α(x)) will be a parameter that reflects the ratio of reflection (or the characteristics of reflection) which is computed by estimating the materials in the 3D model (the ratio between the two values depends on the reflection characteristics of the radiated surfaces but it can be simply selected as a user-defined parameter). In the simplest case it is a value between 0 and 1. The visibility parameter will take the value of either one or zero based on whether the reflected ray reached a target or not, respectively. The other two factors (i.e. distance and angle of incidence) are computed the same way as for direct irradiation considering that the light source is voxel1 (direct target 3D element 268) and the target voxel is voxel2 (indirect target 3D element 270) in the example of FIG. 16.

In the simplest case, one reflection is computed but the reflection computation can be performed for an already reflected beam as well. For each radiated surface element, the direction of the reflected vector is computed according to the direction of the incoming UV-C ray and the local surface normal vector. When the visibility parameter of the surface element is computed, multiple rays are cast, however, for the reflection computation, only one ray is cast to one of the surfaces of the voxel from which it is reflected.

Reflected irradiance estimation is used to compute irradiance more precisely, but it is even more important that some irradiated surface elements can only be reached by reflected UV-C rays. These surfaces are usually covered by other objects thus the UV-C rays cannot reach them directly (i.e. their original visibility value is equal to zero), however, by taking reflected beams into consideration, the invention is preferably able to overcome this limitation. In other words, the irradiation procedure is calculated for places of hidden surfaces by using reflected beams (this is in connection to one type of Learning is to recognize the objects or hidden areas, and calculating the irradiation plan for them).

In the current version, all considered factors are used to estimate irradiance assuming that the light source radiated for certain periods of time in various positions. However, another approach would be to compute the minimum radiation times for the positions, considering all other factors, which are required to have proper disinfection.

In the following, the optimization process for the radiation (disinfection) plan. Some approaches to perform this are illustrated in FIG. 5, where details of operational step S130 for irradiation plan optimization (i.e. radiation plan/radiation plan candidate generation) are given.

In order to obtain an optimized disinfection plan, first the whole irradiation process is encoded in a simpler way. In most cases, assuming that an appropriate 3D model is available and an irradiance evaluation method is defined, a disinfection (radiation) plan can be encoded as a set of radiation positions and corresponding radiation times. This is summarized in operational step S131 of FIG. 5, where it is given that the optimization problem (radiation plan (candidate) generation) is encoded as a set of radiation positions relative to a 3D model of the environment (scene) and the corresponding radiation times which can then be transformed into a 3D model that highlights the irradiance distribution (see also the above description where the manner of the computation of the irradiance is detailed).

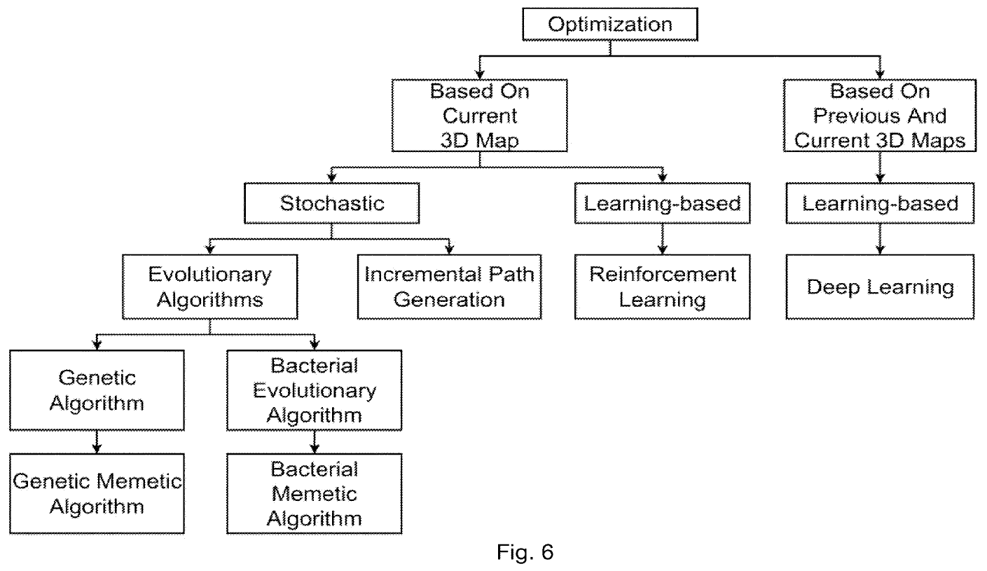

If a mobile robot is used with a fixed light source, then the position simplifies to x and y coordinates but in general the 6DoF pose can be used. Once the problem is encoded, various optimization/plan (candidate) generation methods can be used which are basically artificial intelligence techniques. These methods are classified in FIG. 6 (see below) and detailed in FIGS. 7A-12B. The optimization process provides an optimal radiation (disinfection) plan which is encoded the same way as the original problem (i.e. fits to the 3D model). However, this encoded solution is not the irradiated 3D model (but the radiation plan using only the irradiance ratio estimation). To obtain the 3D model that reflects the irradiance levels on different surfaces the optimized solution is used along with the Irradiance Estimation Formula (see the description of FIG. 1B).

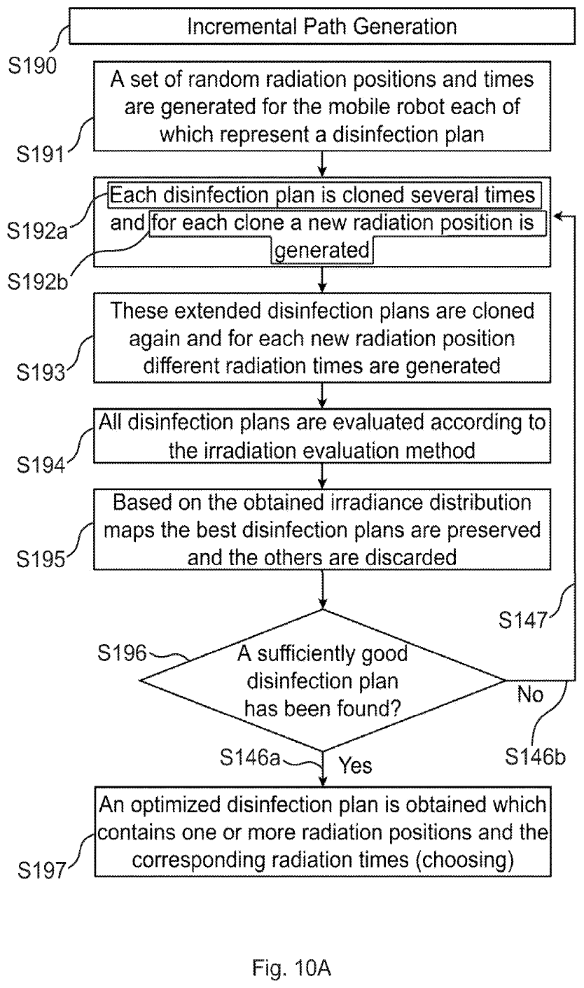

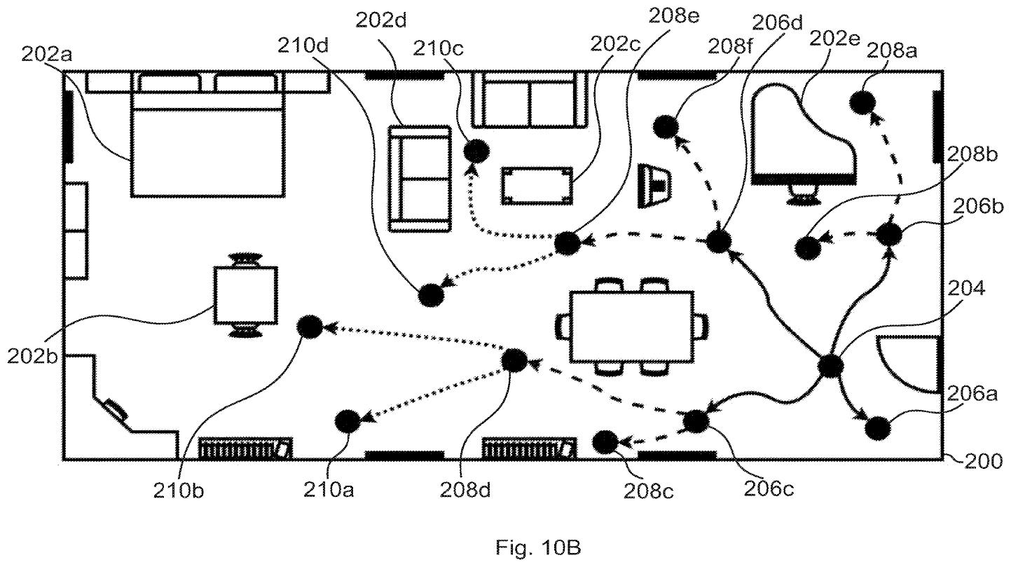

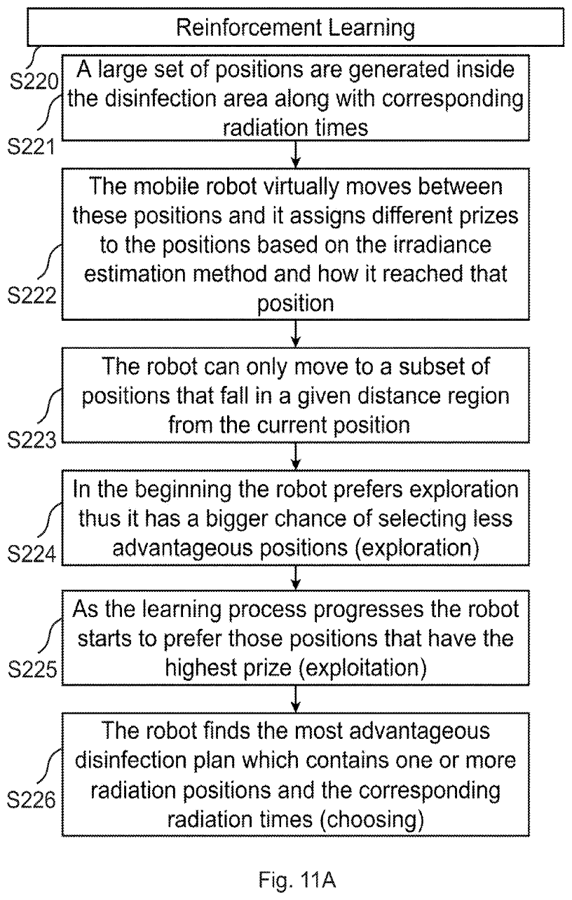

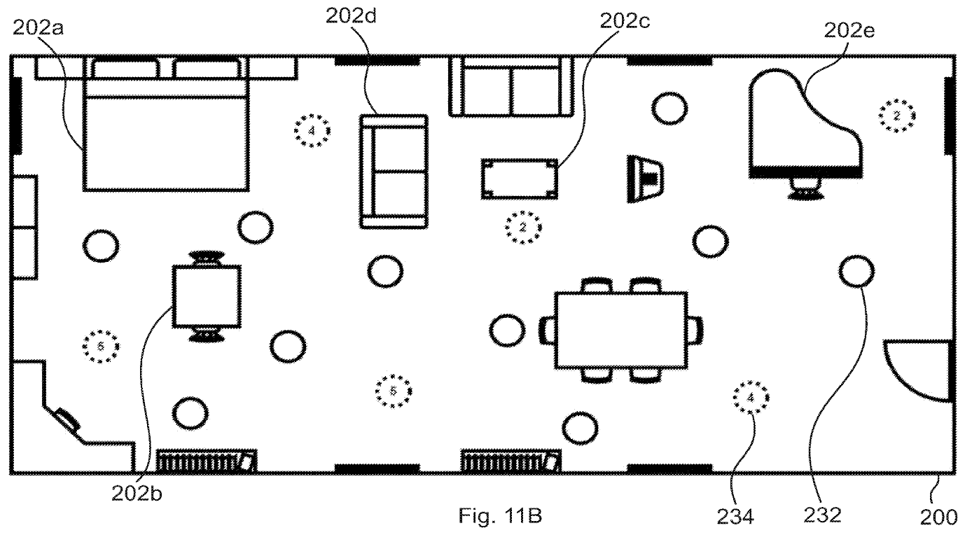

It is emphasised that the invention provides two approaches for the optimization, i.e. for the generation of the radiation plan. The first branch is illustrated by operational step S132a wherein it is given that optimization is based on current 3D map. It is also shown in operational step S132a that the optimization can be performed by means of evolutionary algorithms (see FIGS. 7A-9C), incremental path generation (see FIGS. 10A-10B) and reinforcement learning (see FIGS. 11A-11B), see also FIG. 6 for the classification of the applicable approaches.

The first radiation plan generator module applied in the invention is based on artificial intelligence (i.e. an artificial intelligence technique). It is common in the various embodiments that the artificial intelligence is incorporated in the first radiation plan generator module.

As illustrated in FIG. 6, more particularly the first radiation plan generator module is based on a stochastic or learning-based artificial intelligence (algorithm). Even more particularly, the first radiation plan generator module is based on an evolutionary algorithm, an incremental path generation algorithm or a reinforcement learning algorithm corresponding to respective embodiments of the invention.

It is given herebelow in a nutshell for the latter three realization options what can be considered in each of them to be artificial intelligence related (more details are given below in this aspect for each of these embodiments).

In an evolutionary algorithm based embodiment artificial intelligence is considered to be incorporated into the recombination and mutation steps (the variations of these for different specific evolutionary algorithms, e.g. genetic algorithm and bacterial algorithm, will also be given below) and artificial intelligence may also be utilized in the selection of individuals.

In an incremental path generation algorithm based embodiment, artificial intelligence is considered to be incorporated into manner how extension is made for building the plan candidates, as well as into the selection of the plan candidates the further incremental extension (building) of which will performed.

In a reinforcement learning based embodiment, artificial intelligence is considered to be incorporated into the determination of the weights (i.e. prize parameters), as well as into performing exploration and exploitation steps (phases).

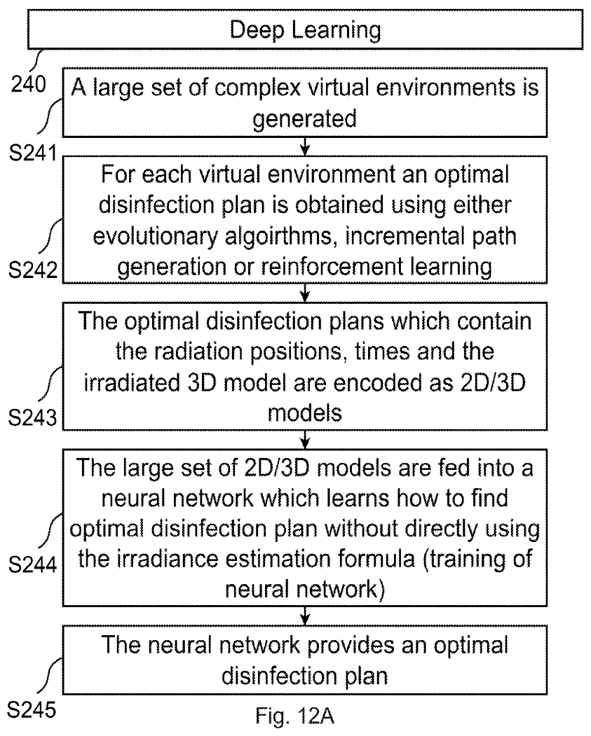

Roughly speaking by the help of these artificial intelligence based approaches (algorithms), the optimization can be performed, i.e. the radiation plan can be determined for any scenes for which the 3D model is generated or is available. On the contrary, the other branch illustrated by operational step S132b is based on different principles. In operational step S132b optimization is based on previous and current 3D maps. It is also given that in this case the optimization is performed by means of deep learning. The details of this approach are given below in connection with FIGS. 12A-12B. Roughly speaking by the help of the deep learning algorithm applied in this branch, the optimization is performed so that in the deep learning algorithm a training is performed using previous optimizations for 3D maps performed by the help of operational step S132a and based on this training the deep learning algorithm becomes to be able to determine a radiation (disinfection) plan directly (i.e. not using implicit irradiance estimation, see below) for a new scene.

The problem of finding optimal radiation positions for an input 3D model by deep learning can be considered as a regression task. The set of input 3D models can be considered as the independent variables, while the optimal radiation positions (can take values between the boundaries of the 3D model) and corresponding radiation times (can be counted for instance in seconds or milliseconds) are the dependent variables (a continuous output and irradiance values are optional inputs during the training).

The task is to train the convolutional neural network so that it can learn the relationship between the independent and dependent variables. To achieve this, a large set of representative training data is required which is obtained through one of the other optimization methods. After the training, the convolutional neural network can compute the required radiation plan if a new 3D model is given as input. The loss function of the system has to be defined so that the difference between the annotated and regressed radiation positions and times is minimal. Furthermore, another element is required in the function which is minimal if the number of annotated and regressed positions is equal. This penalty should increase if the number of regressed positions is higher than the optimal number. Consequently, the neural network is trained to find minimal number of positions that still provide optimal disinfection coverage.

Thus, this structure of operational steps S132a and S132b highlights that although deep learning based optimization is an alternative to the other approaches it requires the results of other techniques as input. The optimal disinfection plans and optionally the corresponding disinfected 3D models that are provided by an optimization method that only uses the current 3D map are used as a dataset (or ground truth) to train a convolutional neural network of the deep learning algorithm. Once the training is finished, the deep learning approach can be used as an alternative solution. It can provide an optimal disinfection plan for a new 3D model presumably much faster than other techniques since a complete optimization process that relies directly on irradiance computation is not required.

It is given in operational step S133 that the result of the optimization process is an optimized disinfection (radiation) plan which contains (comprises) one or more radiation positions and the corresponding radiation times. Referring to FIG. 1B, the process illustrated in FIG. 5 can be concluded that although the optimization process does not directly provide precisely estimated irradiance values for all the 3D elements (mainly since only irradiation ratio estimation is used for optimization), those can be computed according to the irradiance estimation formula using the optimized disinfection (radiation) plan.

In FIG. 6 a hierarchy of optimization methods which can be used in the framework of the invention is shown. The optimization methods that build on artificial intelligence can be divided into stochastic and learning-based algorithms (within the branch entitled “based on current 3D map”). Stochastic methods usually contain many random generations while learning-based methods focus on finding an optimal solution based on previous knowledge about the current problem (reinforcement learning) or previous problems (deep learning; this is taken to a fully different branch as detailed above).

As shown in FIG. 6, the possible optimization methods are first divided into two subsets based on whether
they only need to get the current 3D map or
previously optimized solutions are also required (for training in a phase before a current 3D map is process).

The methods that require previous optimization solutions (second branch) are obviously learning-based methods, more precisely deep learning approaches (those may be divided into convolutional neural networks tackling a regression problem and convolutional graph neural network based methods).

The methods that can provide optimal solutions (first branch) using only the current 3D map may be either learning-based methods, e.g. reinforcement learning, or stochastic optimization methods. In the latter group two approaches are distinguished, the incremental path generation and evolutionary algorithms. Evolutionary algorithms may be further divided into subcategories, e.g. genetic algorithms and bacterial evolutionary algorithms which both have extensions (i.e. memetic algorithms) that contain a further local search phase in addition to the traditional steps of the original algorithms.

In the followings, the various algorithms applicable for optimization, i.e. for generation a radiation plan are described in details. According to the two branches of FIGS. 5 and 6, these algorithms are detailed in FIGS. 7A-11B and FIGS. 12A-12B, respectively.

In FIG. 7A operational steps of evolutionary algorithms—in particular a genetic algorithms—are illustrated (labelled as operational step S160 for plan (candidate) generation by the help of evolutionary algorithm). The optimization problem or more precisely the possible solutions to the optimization problem have to be encoded for instance as a series of floating point numbers, integers, booleans or a combination of these. In this case the problem is encoded as a series of floating point numbers which correspond to radiation positions (2DoF or 6DoF) and radiation times. One or more from these are comprised in a radiation plan (candidate).

Using the phrases of the evolutionary algorithms, a radiation plan (candidate) is called an individual. Moreover, elements in an individual (e.g. a floating point number for a radiation data pair of a radiation position and a corresponding radiation time) are often called genes. A possible solution to the problem is also called individual (since it is also of the same type: a final radiation plan) while the set of possible solutions is commonly referred to as population.

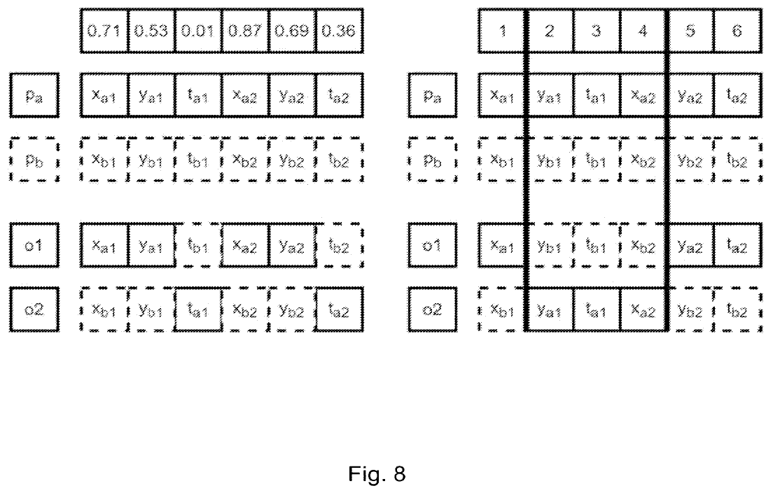

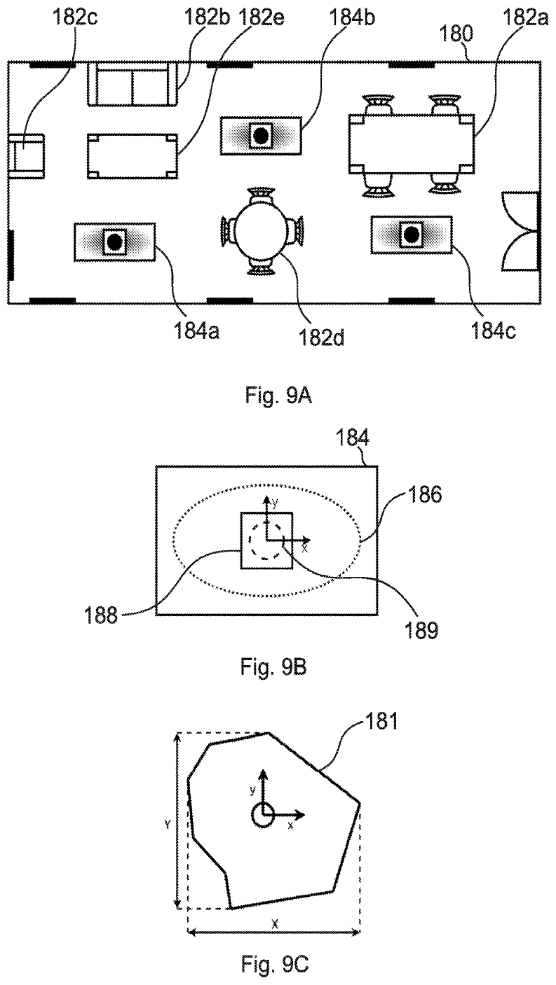

To start the optimization by the help of an evolutionary algorithm an initial population needs to be generated randomly. Then a few steps are iteratively performed to obtain better solutions (see FIGS. 8-9B for the details). Note that these steps can be performed in different orders which depends on the exact type of evolutionary algorithm and the implementation (see also below).

FIG. 7A illustrated mainly by the terms corresponding to a genetic algorithm as an example for evolutionary algorithms; FIG. 7A will be described in details (step by step) below. In this case one of the starting steps of the iteration is an irradiation evaluation (cf. operational step S162) which can be preferably carried out according to FIG. 3. The irradiance estimation block creates a 3D map in which for each element the irradiance value is computed. The possible solutions are then ordered based on how good disinfection can be achieved by them (the best individuals are selected, cf. operational step S163). The quality of disinfection is equivalent to the fitness score. For instance, the ratio of properly disinfected surface elements can be used as the fitness function (cf. with the concept of irradiance ratio estimation). It has to be noted that the above described evaluation and selection phase is used this way only in genetic algorithms. In bacterial evolutionary algorithms, the evaluation and selection operators are parts of the independent modification and recombination steps.

If no sufficient solution is found or a maximum number of iterations is not reached, a subset of the possible solutions or all solutions are then modified independently (mutation, bacterial mutation) and mixed with each other (crossover, gene transfer)—the individuals that are mixed are also called parents—to obtain new and usually better solutions (cf. operational steps S165, S166; about the terms: modified independently→mutant is created; mixed with each other→offspring/child is created). These operators are described by FIG. 8 and FIGS. 9A-9B.

In memetic evolutionary algorithms an optional additional local searching step is present (cf. operational step S167; the embodiment of FIG. 7A comprises this step, wherein a local searching phase is performed to improve each individual by minimizing a cost function). To use this a closed form cost function has to be created that describes the optimization problem. For instance, the Irradiance Estimation Formula can be used for a set of target surface elements to obtain a system of equations, in which the light source coordinates and radiation times are the variables. A local minimum then can be searched by using Levenberg-Marquardt algorithm or other optimization technique. Roughly speaking in this step, the optimization is further enhanced by a local searching phase.

It is note that if the optional operational step S167 is not applied, then the iteration going back to the operational step S162 of irradiance evaluation from the step before, i.e. from operational step S166 (this is also applied equivalently to the embodiment of FIG. 7B for operational step S167′).

In the process illustrated in FIG. 7A, the iteration continues until a sufficiently good solution is found or a predefined maximum iteration number is reached (cf. operational steps S164a and S164b). The output of the optimization process is a single solution that is encoded the same way as each possible solution (i.e. individual). As mentioned above, the decision has to be made after evaluation and selection.

Thus, in an embodiment—being in connection with FIG. 7A but more general in many aspects —, the first radiation plan generator module is established by means of an evolutionary algorithm, at least two plan candidates are generated in the plan candidate generating step (i.e. it is specified here that within the above introduced plan candidate generating step not one or more—this is overwritten —, but specifically at least two plan candidates are generated, since at least two of these are needed to perform the specific steps of the evolutionary algorithm), each of the at least two plan candidates has the same radiation data pair number of radiation data pair, a total radiation time is obtained for each plan candidate by summing up the corresponding one or more radiation time for all of the one or more radiation position, and a first condition of the radiation plan sufficiency criterion is applied in an irradiation iteration having at least one irradiation iteration cycle, wherein in case of fulfilling the first condition (one of the following two options is selected),
the first radiation plan is obtained in the radiation plan choosing step,
   in case the radiation plan sufficiency criterion is based on the radiation time limit, from the at least two plan candidates having a total radiation time below or equal to the radiation time limit, as the plan candidate having the highest irradiance ratio estimation (‘the highest’ is introduced by definite article, since the highest one is always definite), and
   in case the radiation plan sufficiency criterion is based on the irradiance limit, from the at least two plan candidates having an irradiance ratio estimation above or equal to the irradiance limit, as the plan candidate having the lowest total radiation time, or (this ‘or’ corresponds to the relationship between the first option—having two cases—and the next option)
in case of fulfilling the first condition in the first of the at least one iteration cycle (this option can only be selected in this highly specific case of early (i.e. for the first time/occasion) fulfilment of the first condition in the first iteration cycle), the radiation data pair number is decreased by one, if it is above one, and the irradiation iteration is restarted.

In a further embodiment related to the above a second condition of reaching of a predetermined maximum iteration number is applied in case the first condition is not fulfilled, wherein in case of fulfilling the second condition (one of the following two options is selected also in this case)
the first radiation plan is obtained in the radiation plan choosing step as the plan candidate having the highest efficiency proportion of the irradiance ratio estimation and the total radiation time, or
the radiation data pair number is increased by one and the irradiation iteration is restarted.

The second condition is only investigated—for such cases—when the first condition is not fulfilled, if there is a second condition at all; cf. FIGS. 7A-7B. The role of the second condition is to stop the iteration when the sufficiently good solution cannot be reached (e.g. since the limit conditions for that are too high) and it helps to reach a “good enough” solution. The predetermined maximum iteration number is an input of the method, it is selected in a way so that to let to search a sufficiently good solution for enough iteration cycles but not for too much iteration cycles.

The radiation data pair number is thus the same for all of the at least two plan candidates. Accordingly, this number can be used also separately for them, because it is the same for all of them.

In a further embodiment built on the above embodiments introducing the first and second conditions (embodiment of FIG. 7A is such an embodiment, but in the present level, many kind of evolutionary algorithms are covered, e.g. also bacterial evolutionary algorithm, not only genetic algorithm, see also below, where these two kind of algorithms will be specified in further embodiments), after generating the at least two plan candidates in the plan candidate generating step (cf. operational step S161, the plan candidates are prepared for the iteration steps), in course of an iteration cycle (cf. operational step S142 which is illustrated by an arrow coming back from the last step of the iteration cycle) of a first irradiation iteration
checking a fulfilment of the first condition for the at least two plan candidates and, if the first condition is not fulfilled, a fulfilment of the second condition (cf. operational step S164 where such a hierarchy of the conditions is illustrated in operational steps S164a, S164b),
   in case the first condition or the second condition is fulfilled (cf. operational step S141a—“yes” branch—illustrated by an arrow from operational step S164 of checking to operational step S168 of radiation plan choosing), the first radiation plan is obtained in the radiation plan choosing step (the number of the plan candidates is fixed throughout the iteration cycles—if not decreased after the first cycle —, since this obtaining the first radiation plan option is chosen from the above choices, increasing of the radiation data pair number is not allowed in this embodiment, for an example letting also increasing option see an embodiment below in connection with FIG. 7B),
   in case neither the first condition nor the second condition is fulfilled (cf. operational step S141b—“no” branch—where the iteration continues with the special evolutions of the plan candidates by means of the applied evolutionary algorithm), at least two plan candidates of a next iteration cycle are generated through an arbitrary order application of recombination (cf. operational step S165) and/or mutation (cf. operational step S166; these are general recombination and mutation for all type of evolutionary algorithms, see below for options where these are specified) of the at least two plan candidates (the next at least two plan candidates are generated for the next iteration cycle based on the actual at least two plan candidates cf. operational step S142 illustrating the iteration cycle),
      wherein a respective irradiance ratio estimation in the irradiation evaluation step is generated for each of the at least two plan candidates during each iteration cycle (in this case the exact place of the irradiation evolution in the algorithm is not specified since it can be various for different evolutionary algorithms, cf. the next embodiment and operational step S162 of FIG. 7A for an exact place of this step).

An interpretation is given in connection with the step ‘the first—i.e. final—radiation plan is obtained in the radiation plan choosing step’. This step is performed in case the first condition or the second condition is fulfilled and according to which one of the conditions is fulfilled, since as given above, the manner of obtaining depends on which condition is fulfilled. Above, it is specified, that how the (final) radiation plan is obtained in the separate cases of fulfilment of each condition (for the first condition in dependence of the type of the limit specified).

In a further embodiment the evolutionary algorithm is a genetic algorithm, the recombination is a crossover and the mutation is a mutation according to the genetic algorithm, and in course of the first irradiation iteration cycle the steps are ordered as (i.e. the above introduced steps have the following order)
generating a respective irradiance ratio estimation in the irradiation evaluation step for each of the at least two plan candidates (cf. operational step S162 of FIG. 7A), and
checking a fulfilment of the first condition and, if the first condition is not fulfilled, a fulfilment of the second condition (cf. operational step S164 of FIG. 7A).

This embodiment is in line with the embodiment of FIG. 7A, but this latter is still further specified. In the embodiment of FIG. 7A, a truncation selection is performed in operational step S163 (just after the evaluation in operational step S162), wherein the best individuals (plan candidates) are selected based on their fitness score (irradiance ratio estimation) that expresses how good a certain disinfection plan is (the truncation selection can be performed at this point, since a sufficiently good solution—if there is—will be among the best individuals).

Furthermore, in the embodiment of FIG. 7A, some more specialities are given for recombination and mutation (independent modification) and their order is fixed (their order could be inversed also). Thus, in operational step S165 new individuals are generated through the recombination of the genes of the previously (truncation) selected individuals. After that, in operational step S166 the original and new individuals are further evolved independently of each other by modifying some of their genes.

In an embodiment being an alternative of the previous embodiment (where the evolutionary algorithm is a genetic algorithm)
the evolutionary algorithm is a bacterial evolutionary algorithm, the recombination is a gene transfer and the mutation is a bacterial mutation, and
the respective irradiance ratio estimation in the irradiation evaluation step is generated for each of the at least two plan candidates within the gene transfer and/or the bacterial mutation (see below for the details for applying such evaluation within these steps).

According to FIG. 7B, before the evolutionary algorithm starts the optimization, the minimum number of radiation positions is estimated based on the size of the area to be disinfected (cf. operational step S171). In the approach of FIG. 7B the number of radiation positions is not fixed, but it is increased if a maximum number of iterations is reached for a given number of radiation positions and no sufficiently good solution is found.

After the best individuals are selected it is checked whether a sufficiently good solution has been found (cf. operational step S172). If so, then the optimization is finished and an optimized disinfection plan is obtained (cf. the last operational step S168′). If the solution is not good enough but the maximum number of iterations has not been reached, then the algorithm continues with the next iteration (recombination and modification follow).

However, if the maximum number of iterations is reached but a sufficiently good solution could not be found then the number of radiation positions is increased (this number can be increased by one or more radiation positions based on the deviation from the ideal irradiance level; i.e. more radiation position may be added in at the same time; cf. operational step S174). This process continues until a sufficiently good solution is found. Similarly, if a sufficiently good disinfection plan could be obtained using the estimated minimum number of radiation positions then the algorithm may attempt to reduce this number and check whether a smaller set of radiation positions is still sufficient.

In an embodiment corresponding to FIG. 7B (but being in a higher generality level than that), the first radiation plan generator module is established by means of a genetic algorithm, and, in course of an iteration cycle (cf. operational step S145, on which the whole iteration procedure is meant with two iteration starting step in it, i.e. two points to which a previous iteration can get back) of a second irradiation iteration
at least two plan candidates are generated in the plan candidate generating step (cf. operational step S161′, which is formulated more specifically in FIG. 7B: a set of individuals are created each of which consists of, i.e. comprises in this case, genes that encode radiation positions and radiation times, see also operational step S161 of FIG. 7A) as a first iteration starting step (this will be the step to where the outer loop of the iteration comes back, illustrated by an arrow of operational step S145a),
generating a respective irradiance ratio estimation in the irradiation evaluation step (cf. operational step S162′, see also the similar step in FIG. 7A) as a second iteration starting step (i.e. the irradiation evolution step plays the role of the second iteration starting step for the inner loop, illustrated by an arrow of operational step S145b) for each of the at least two plan candidates,
checking a fulfilment of the first condition of the radiation plan sufficiency criterion (cf. operational step S172) for the at least two plan candidates,
   in case the first condition is fulfilled (‘yes’ branch in operational step S143a), the first radiation plan is obtained in the radiation plan choosing step (cf. operational step S168′, see also the similar step in FIG. 7A),
   in case the first condition is not fulfilled (‘no’ branch in operational step S143b), checking a fulfilment of the second condition,
      in case
         the second condition is fulfilled (cf. operational step S144a) and,
         when the second condition is fulfilled for at least the second time, a first auxiliary proportion of
         an actual highest efficiency proportion of the irradiance ratio estimation and the total radiation time of the at least two plan candidates and
         the previous first efficiency proportion parameter is above a first predetermined development parameter, the highest efficiency proportion of the irradiance ratio estimation and the total radiation time of the at least two plan candidates is given to a previous first efficiency proportion parameter and the radiation data pair number is increased by one (cf. operational step S174, where this is termed as the number of radiation positions is increased; naturally, also a radiation time corresponds to the newly generated radiation position) for a next iteration cycle continued at the first iteration starting step (see outer loop of the iteration in operational step S145a),
      in case
         the second condition is fulfilled and,
         when the second condition is fulfilled for at least the second time, the first auxiliary proportion of
         an actual highest efficiency proportion of the irradiance ratio estimation and the total radiation time of the at least two plan candidates and
         the previous first efficiency proportion parameter is below the first predetermined development parameter,
      the first radiation plan is obtained as the plan candidate having the highest efficiency proportion of the irradiance ratio estimation and the total radiation time,
      in case the second condition is not fulfilled (‘no’ branch in operational step S144b), the at least two plan candidates are generated for a next iteration cycle continued at the second iteration starting step (see inner loop of the iteration in operational step S145b) through crossover (cf. operational step S165′) and/or mutation (cf. operational step S166′) according to the genetic algorithm of the at least two plan candidates.

According to the further specification of FIG. 7B, after operational step S162′, a truncation selection is done, wherein the best individuals are selected based on their fitness score (irradiance ratio estimation) that expresses how good a certain disinfection plan is.

In FIG. 7B the operational step S165′ corresponding to crossover is termed as new individuals are generated through the recombination of the genes of the previously selected individuals. Moreover, in FIG. 7B the operational step S166′ corresponding to mutation is termed as the original and new individuals are further evolved independently of each other by modifying some of their genes.

In a nutshell, the cases given above for checking a fulfilment of the second condition:
if the second condition is fulfilled and the new candidates can develop sufficiently (this is measured by the first auxiliary proportion by the help of the first predetermined development parameter) compared to the case with radiation positions having a number smaller by one (this is why this condition is checked when the second condition is fulfilled for at least the second time), then the actual value of the efficiency proportion is stored as a previous value (for the next such cycle), the radiation data pair number is increased by one and the iteration continues in the outer loop;
if the second condition is fulfilled and the new candidates cannot develop sufficiently, the (final) radiation plan is obtained according to the highest efficiency proportion and the iteration is terminated;
if the second condition is not fulfilled, a new set of at least two plan candidates is generated using crossover and/or mutation and the iteration continues in the inner loop.

While disinfection is being performed, the mobile robot that carries the UV-C lamp might move constantly or stop in certain positions to radiate (as it was mention above, a ray-tracing methodology to calculate the irradiation in 3D for an UV-C lighting robot along a pre-defined or automatically driven path). Although, for smaller 3D environment models the created algorithm is able to estimate irradiance in real time, the movement of the robot would make calculations much more difficult, as the position of the lamp relative to the voxels would change constantly. Instead, according to the invention, it is assumed that the robot moves to one or more (typically, several) target positions and in each of which it stops to radiate for a given period. One type of Path optimization is based on stochastic optimization, e.g. Active Contours/Splines, Graph optimization, Markov Random Fields.

However, on the one hand, the random selection of these radiation positions can easily lead to inferior performance, while on the other hand, manual selection would require human interaction thus the disinfection task could not be accomplished autonomously. To solve these problems the candidate and the final radiation plans are generated by the help of artificial intelligence. Herebelow, a genetic algorithm based optimization method is described in details.

The main concept of genetic algorithms is the following. First, an initial population has to be created in which each individual encodes a combination of optimization parameters (thus optimization parameters, i.e. the parameters to be optimized: the radiation data pairs, the combination of which gives—in the role of an individual—a radiation plan (candidate)). Each individual is evaluated according to a predefined function and the best solutions to the optimization problem are selected to survive. After that, new individuals are created using biologically inspired operators such as crossover and mutation. The selection, crossover and mutation steps are repeated until, for instance, a fixed number of generations is reached or a sufficiently good solution is found.

In the proposed algorithm, the size of an individual depends on the required number of radiation positions nₚ which can be set as an input parameter. Before the initial population can be created, the borders of the 3D model have to be computed and a radiation time limit tₗᵢₘᵢₜ needs to be defined. The latter is used to limit the total duration of the disinfection process. Each individual in the initial population encodes the x and y coordinates of the lamp positions and the corresponding radiation times. Consequently, an individual consists of 3nₚ genes (see the description of FIG. 8). All genes are randomly selected with two criteria, every lamp position needs to be within the borders of the 3D model and the sum of the radiation times may be expected to be equal to tₗᵢₘᵢₜ (e.g. when irradiation limit is expected) or may be expected to be under this limit.

In the following some details are given in connection with Genetic Algorithm Evaluation and Selection. Note that if only every nth element is considered in the 3D model for direct irradiance estimation, then presumably the irradiance ratio values will be approximately one-nth of the real values, thus the limit for the sufficiently good solution has to be adjusted accordingly. After Pᵢ(see above) has been computed for all individuals (radiation plan candidates) if a sufficiently good solution could not be found (by applying a strict criteria), then a selection step is performed in which the individuals for the next iteration are selected (cf. operational step S163 of FIG. 7A; the recombinations and modifications are to be based on these individuals if these are still needed since the iteration is not ended in operational step S164).

For this selection, different selection methods can be used (e.g. truncation selection, roulette wheel selection, tournament selection) with truncation selection being the most simple (this is utilized in FIG. 7A). Note that totally random selection is not an option since it is desired to preserve the better individuals in each iteration.

A subset or all of these individuals then can be used as the input for recombination and independent modification. The specific inputs will be selected in the beginning of the recombination and independent modification steps. As can be seen in the first selection step the output set of the previous iteration is narrowed down to a smaller subset. In contrast, in the latter selection steps specific individuals are selected from this smaller subset to be either parents or base individuals of the independent modification step. In this second case of selections, similar techniques can be used, even totally random selection, however as truncation selection directly takes a subset, not one-by-one, it is not applicable.

In truncation selection the radiation plan candidates are sorted based on their fitness score and the best solutions are selected to survive. The roulette wheel selection (i.e. fitness proportionate selection) first normalizes the fitness values so that their sum is equal to one, then the individuals are sorted in descending order by their fitness values, thus to each individual belongs an interval between 0 and 1 which is proportional to its fitness value. After that, a random number is generated between 0 and 1 and after checking which interval the random number falls within, the corresponding individual is selected as a parent. If tournament selection is applied, then subsets of the whole initial population are selected randomly (the size of the sets, i.e. the number of elements in a set can be defined as an input parameter) and the individuals with the highest fitness values in each set are selected as parents.

Implementation and concept of two crossover (i.e. recombination) types are illustrated in FIG. 8. In both cases two parent individuals are selected randomly and their genes are mixed to obtain two children.

In FIG. 8 the concept of uniform (left) and two-point (right) crossover in genetic algorithms are illustrated. The crossover operator in genetic algorithms is used to obtain better solutions from the already available ones through recombining them (the word crossover is used for recombination for genetic algorithms, recombination is the generalized term for any evolutionary algorithm).

In FIG. 8, the squares represent either x or y coordinates or irradiation times. When uniform crossover is performed (left of FIG. 8), for each position in the input solutions (1 . . . 6 in this example) a random number is generated between 0 and 1. Based on whether the number is greater or smaller than 0.5 the first new solution gets the gene at the given position from the first or the second input solution respectively. The second new solution is the exact opposite of the first which holds true for the two-point crossover as well. However, the selection of genes is different in two-point crossover (right of FIG. 8). In that case, two positions are randomly selected (from 1 . . . 6 in this example) and the genes that do not fall between these positions are transferred from the first input solution to the first new solution. All other genes are provided by the second input solution.

The input individuals of the crossover operator are also called parents thus the first and second selected input individuals are denoted in FIG. 8 by pₐ and p_{b} respectively. The outputs of the crossover operator in accordance with the aforementioned convention are usually called offsprings (sometimes they are referred to as children) thus they are denoted by o1 and o2.

Since each individual encodes a certain number of radiation positions in 2D (this pose may be extended to 6D) and the corresponding radiation times, the number of genes in them is a multiple of three. Currently the genes are in the following order: x₁, y₁, t₁, x₂, y₂, t₂, . . . , xₙ, yₙ, tₙ where n is the number of radiation positions. Note that the genes could be organized in any other order, but it is important that the order of the genes has to be the same for all individuals during the optimization. Although the corresponding name of the genes are written in the squares, to further highlight which genes belong to the first and second parents in the offspring, they are denoted by solid and dashed lined squares respectively. The radiation time parameters are similar to the x and y coordinates in the sense that they are crossed and mutated the same way.

The number of crossovers per generation can also be set as an input parameter. There are other crossover operators which can also be used, for instance, the n point crossover is the generalization of the two point crossover. In n point crossover, the two parent individuals are divided into continuous sections according to the n crossover points. The crossover points serve as the boundaries of the sections, where each crossover point corresponds to a gene and belongs to exactly one section. Note that the two parents are divided the same way. Then the first offspring is composed by alternately selecting a section from the first and the second parent. The second offspring is composed so that it is the exact opposite of the first one.

The mutation step can also be performed in two ways. The lamp positions and radiation times in the original individual serve as the references (the centre of the probability distribution) and the genes in the mutant are computed according to either normal or uniform distribution (any continuous probability distribution can be used which has a mean value). Consequently, the new lamp coordinates are selected within rectangles around the original positions as illustrated in FIGS. 9A-9B. The sides of these rectangles are proportional to the dimensions of the room.

The area around the original radiation position, inside which the new radiation position is obtained, can be set to fixed size, however it is often more advantageous to use information about the shape and size of environment to adjust the dimensions of this area. The basic shape of a room or corridor is usually rectangular thus two perpendicular dimensions can be obtained which are used to define the area around the original radiation position.

If the mutation is performed according to uniform distribution, then the area under consideration will take rectangular shape. The dimensions of this rectangular area are proportional to the two perpendicular dimensions of the room (e.g. the factor of proportionality can take value between 0.02 and 0.1) and the new radiation position is selected totally randomly inside this territory.

In the case of normal distribution, this rectangular shape can also be used, however, the selection of the new radiation position is more complicated. Normal distributions do not have really strict borders, however, it can be assumed that in the one dimensional case the new value will be obtained between −3 sigma and +3 sigma (this corresponds to the rectangle, i.e. through the determination of the rectangle, the deviation (sigma) is also defined; sigma may be different in the two directions to have a rectangle), where sigma is the parameter of the distribution and its value can be set according to the desired factor of proportionality and the dimensions of the environment.

The resulting area for the two perpendicular dimensions will be rectangular, however, the new radiation position will most likely be obtained inside an ellipsoidal area 186 around the original radiation position (within the circle corresponding to lamp 189, see FIG. 9B). Furthermore, the uneven colouring of the ellipse in FIG. 9A shows that the new radiation position has a higher chance to be selected closer to the original one.

FIGS. 9A-9B illustrate mutation that is an operator in genetic algorithms by which the input individuals are modified independently of each other. The main concept of mutation is that a set of selected genes (i.e. coordinates of radiation positions and radiation times) are changed in a set of selected individuals (i.e. disinfection plans). If the obtained new solution is better (i.e. has a higher fitness score) than the original one, the new individual will most likely survive. However, if its fitness score is lower than it may not be preserved. In FIGS. 9A-9C the position mutation is mainly illustrated since it is easier to visualize however it is also given how both positions and times are mutated in the case of gaussian distribution based mutation.

Accordingly, FIG. 9A illustrates the concept of position mutation in the presented genetic algorithm. The new coordinates are selected within rectangles (see rectangles 184a-184c, as well as rectangle 184 in FIG. 9B) around the original positions according to either normal or uniform distribution. The rectangles are thus generally computed based on the dimensions of the room (cf. a scene 180—which is actually a room—and rectangles 184a-184c).

In FIG. 9A a room is shown as scene 180, in which several furnitures 182a-182e are arranged. The mutation operator in genetic algorithms is used to obtain better solutions through individually modifying the already available solutions according to a predefined “policy”. In this case, the policy is to change the position of the robot according to a probability distribution function where the available new positions somewhat represent the dimensions of the room. This assumption is used for instance to handle long corridors, but can be used in special-formed rooms as well since only an x and y dimension is required to describe the dimensions of the rectangle (see the description of FIG. 9C below). The mutation for the disinfection times also follows a probability distribution function for which the deviation is proportional to the overall disinfection time.

As it can be seen in FIG. 9B, the environment of a lamp 189 in 2D was cropped from FIG. 9A (also a base 188 corresponding to the lamp 189 is shown). Assuming that in a selected individual all three genes that belong to the first radiation position-time pair (generally, a radiation data pair) were selected for mutation, the genes are modified as follows.

First, the three corresponding genes, i.e., x₁-y₁-t₁ can be considered as the 3D representation of a disinfection “subplan” (i.e. that part of a disinfection plan which belongs to one radiation position and the corresponding radiation time) of where the 3D contains the 2D space and the time dimension. The three genes are then mutated according to the same type of probability distribution function. For instance, they can be mutated according to three different normal (gaussian) distribution functions for which the means are the x₁-y₁-t₁ values. The deviations or sigma parameters of these distributions are computed as follows.

For the x and y coordinates these values are selected so that the new x and y coordinates certainly fall into a rectangle that is proportional to the size of the room. More precisely, the x and y coordinates are mutated separately and the new values will be generated on a section along the x and y axis, which results in a new 2D position generated inside a rectangle. Note that although in theory the new 2D position may fall anywhere inside the rectangle it will most likely be generated inside the dashed ellipsoidal area 186 around the lamp 189 (according to the gaussian decay, it has an ellipse like envelope; the gradient colouring illustrates in FIG. 9A that it is more likely to find a new 2D position closer to the original 2D pose; FIG. 9B shows only the envelope).

The radiation time is also mutated similarly according to (in this case) a gaussian distribution. In the beginning, each radiation time is initialized and the initial value may be a base radiation time (for instance 3 minutes) or may be generated to be in an interval (for instance between 2 and 4 minutes). Then during the first modification, this initial value will serve as the mean of the gaussian distribution (in subsequent iterations, the mean will be the current radiation time of the input individual).

The deviation parameter (see above for sigma) is selected according to the will of the user, for instance it can be set in a way that the change in the radiation time cannot exceed 1 minute in a single iteration.

All in all, the three genes are mutated very similarly, only the means and the deviation parameters are selected based on different assumptions. It has to be noted though, that the genes to be mutated are selected randomly. Consequently, it may very well happen that not all three corresponding genes are mutated in a single iteration just for instance the x coordinate and the radiation time.

Although, the environment may be complex, the algorithm requires only two perpendicular dimensions of the room that can be used to determine the deviations of the probability distributions (for the x and y coordinates). This in practice will result in a rectangle however the orientation of this rectangle may vary based on how which dimensions of the room were selected. This assumption about the environment is advantageous for instance when a corridor needs to be disinfected. The robot will have a much higher chance to move a bigger step parallel to the wall than perpendicular to it.

This is illustrated by the help of FIG. 9C for a room 181 having a more complex shape. In this case, there are also specific x-y directions for which the X and Y size of the room 181 can be determined. These X and Y sizes will be proportional with the deviation of modifying in x-y directions thus defining a rectangle also in this case.

The radiation times can also change, however, their sum still has to be equal to tₗᵢₘᵢₜ. Since it is randomly decided whether a gene mutates or not, some lamp positions and radiation times might remain the same as in the original individual.

If the sum of radiation times in a radiation plan would exceed the predefined t_limit, the times that belongs to the radiation positions are normalized (see also above where the normalization was touched). Let us assume that the limit is t_limit and the obtained times are t1, t2, t3 for which t1+t2+t3>t_limit, then the radiation times are modified as follows: t1′=t_limit*t1/(t1+t2+t3) (t2′ and t3′ similarly) (t1′+t2′+t3′=t_limit). Alternatively, if the limit is not that strict, then the radiation plan can be penalized by multiplying the fitness score by (t_limit/(x+y+z)).

The mutation operator can be applied to all of the individuals in the current population or just to a subset of it. In the latter case, the individuals to be mutated can be selected similarly to the selection method in crossover operators (i.e. totally random selection, fitness proportionate selection, tournament selection).

The second sentence of the above section is important in the context that whether only the modified individuals survive (or a subset) after the modification in a certain iteration or the original individuals as well. In the first case, since the genes to be modified are selected randomly, some original individuals may survive as the “modified” individual will be identical if no genes were selected. In the second case, it is important to handle duplications.

The third and fourth sentences of the above section are important in the context of computational demand. The evaluation of a few individuals does not require a lot of time but for a large group of individuals it may take a significant amount of time. If the time of optimization is not an important factor, then all original and children (i.e. offspring) individual can be mutated. However, if the resources are limited then it may be advantageous to only mutate relatively good solutions. Note that in the latter case a subset of individuals can also be selected randomly regardless of the fitness scores.

In order to decide whether a sufficiently good radiation plan was found, the individuals have to be evaluated by the irradiance estimation formula in each iteration to obtain the ratio of properly disinfected surface elements which results in a fitness score (cf. operational steps S162 and S163 in FIG. 7A, once the we get back to these). The fitness score of an individual in genetic algorithms shows how good a solution is to the particular problem, where the problem is formulated as a so-called fitness function. The most important requirements for a fitness function are that it should represent the optimization problem in a mathematical form, and the obtained fitness scores should serve as the basis for ranking the individuals.

More precisely, the fitness score of an individual (radiation plan) is equal to the number of voxels for which the irradiance is higher than Iₗᵢₘᵢₜ divided by the total number of voxels (i.e. this is the definition of the irradiance ratio estimation). It is noted that the concept of fitness score originates from the evolutionary algorithms, that is why it is generally called irradiance ratio estimation throughout this description. However, based on the above definition it might be generally called as fitness score. The irradiance ratio estimation is a scalar value corresponding to the whole 3D model: it specifies how many of the 3D elements are irradiated to the sufficient limit (this is binary: a 3D element is sufficiently irradiated or not).

However, the irradiance level of each of the 3D elements may also be a significant parameter and it can be relevant in irradiance monitoring (visualisation, see e.g. FIGS. 19A-19H, 20A-20E). Also, an irradiance average can be calculated based on these data which may even help in adjusting the Iₗᵢₘᵢₜ value itself (e.g. when the irradiance ratio estimation cannot reach the limit Iₗᵢₘᵢₜ).

It has to be noted that several cells are added to the model to improve visualization, as it was mentioned above in connection with the environment reconstruction, however, these additional elements are preferably not taken into consideration during the selection.

A Pᵢ score of an individual (i.e. a radiation plan or a radiation plan candidate; each of these has a respective Pᵢ score, which is thus the fitness score of an individual, and, therefore, gives the value of the irradiance ratio estimation for that radiation plan candidate) is described by the following equation, where m denotes the total number of voxels and Iⱼ denotes the irradiance value of the jth cell, respectively (see also the approach, where every n^{th} 3D element is taken into account). Furthermore, Iₗᵢₘᵢₜ denotes an acceptable (pre-determined/pre-defined) level of irradiation.

$${P}_{i} = \frac{{\sum }_{j = 1}^{m} {δ}_{j}}{m} \qquad {δ}_{j} = \{ \begin{matrix}1 , if {I}_{j} > {I}_{limit} \\ 0 , if {I}_{j} \leq {I}_{limit}\end{matrix}$$

In an embodiment, after Pᵢ has been computed for all individuals (radiation plan candidates), they are sorted based on their score and the best solutions are selected to be the initial population of the next generation (cf. operational step S163 of FIG. 7A; the recombinations and modifications are to be based on these individuals if these are still needed since the iteration is not ended in operational step S164). This selection method is called “truncation selection” and the number of individuals to survive can be set as an input parameter (other selections are also possible, like roulette or tournament).

As an alternative to the traditional genetic algorithm, a bacterial evolutionary algorithm can also be implemented as an evolutionary algorithm. In this case, the encoding of the individuals and the random generation of the initial population are performed the same way as in the traditional genetic algorithm. However, the type and the order of the applied operators are different.

There is an important difference between genetic algorithms (GA) and bacterial evolutionary algorithms (BEA) regarding the selection.

As it was mentioned above there are two kinds of selections, the first one is responsible for preserving the better individuals while the second is responsible for selecting those individuals from the current population that should be evolved. In GA, the first selection is performed as follows. The population in the beginning contains ‘b’ elements, then after recombination, ‘b+c’ elements and after mutation ‘b+c+d’ elements (since, original individuals are preserved). The first selection step considers the ‘b+c+d’ element set collectively (after collective evaluation) and selects ‘b’ better/best elements.

In contrast, in BEA when an individual is selected for bacterial mutation (or two for gene transfer), the obtained new individual(s) are compared with the original one and the best is selected (after the new individuals are evaluated as well). Then the algorithm continues the bacterial mutation step (or gene transfer) with the next individual(s).

This way, in BEA the first kind of selection is performed inside other steps of the iteration. Note that in this case (BEA), the above detailed selection methods are not used since it becomes a strict decision to choose the best individual (a very concrete case of truncation where only the best one is preserved). Accordingly, after the bacterial mutation and gene transfer, individuals of the same number remains, no dropout of individuals is needed.

Although the above described first kind selection approach for GA is commonly used, it is possible to use the one which was described for BEA, i.e. the mutant or the original individual survives or two individuals from two parents and the children. In this case, the dedicated selection step does not need to be used, but the evaluation has to be performed inside the crossover/mutation step).

The second kind of selection is more similar in the two algorithms. In GA both the number of crossovers and the number of mutations (how many of them should be performed in each iteration) are set as input parameters and the above mentioned selection steps can be used accordingly. In the case of BEA, the bacterial mutation usually considers all individuals thus no second type selection is required. The number of gene transfers in an iteration can be set however, and the input individuals are usually selected randomly. In theory, the other second type selection methods can be used as well, however as the individuals are already separated into better and worse ones, it may be enough to select randomly. The separation of good and bad individuals has to be performed after each gene transfer.

First, the individuals are modified independently using a bacterial mutation operator (see herebelow). The number of individuals for which bacterial mutation is applied can also be limited but usually every individual is used. For each of them, a certain number of clones are generated (the number of the clones can be set as a pre-determined input parameter). Then the individual and the clones are divided into segments (the segments are the same for all of them).

A segment in an individual may contain any number of genes (even 1) and the segment is not necessarily continuous. Consequently, a segment does not correspond to a radiation position-time pair, it may consist of the following genes, for instance: x1, y2, t5. The segments are modified according to FIG. 9A-9B. In the bacterial mutation all input individuals are considered one-by-one and in each of them all segments are examined.

One segment is selected at a time, and all clones modify that segment differently, in a similar way as for the traditional genetic algorithms (using different probability distributions). All clones are evaluated based on the fitness function and the modified segment of the best individual is copied to every clone. Then a new segment is selected until all of them have been mutated. At the end of the modification of the individual some of its segments may have changed, some did not. Once the first individual is modified, the bacterial mutation continues with the next individual.

The bacterial mutation is followed by the gene transfer operator (i.e. recombination). The individuals are sorted based on their fitness values and divided into a “good” and a “bad” half. Then a “good” and a “bad” individual is selected randomly along with a segment in them and the segment in the “bad” individual is replaced by the segment in the “good” individual. Preferably the modified “bad” and the original “bad” individuals are compared and the one with the higher fitness score is preserved (optionally the modified individual can be preserved regardless of the fitness scores). This process is called infection, and the number of infections determines how many good-bad individual pairs should be selected. Then the new individuals are evaluated and the order of the population is recalculated. The number of infections can also be set as an input parameter. The bacterial mutation and gene transfer are usually performed in this order however, in theory their order may be switched.

In bacterial evolutionary algorithms, the evaluation is completely built in the two applied operators, thus a dedicated selection step is not used. Consequently, the number of individuals in the current population does not change during the optimization. The bacterial algorithms often converge faster compared to the traditional genetic algorithms. The optimization stops when a maximum number of iterations has been reached or a sufficiently good solution has been found.

The number of maximum iterations and the sufficiently good solution can be defined for bacterial evolutionary algorithms as well thus the number of radiation positions can be potentially increased similarly to genetic algorithms. Although the sufficiently good solution criteria is checked after bacterial mutation and gene transfer have been performed, technically it could be checked during the execution of either step since the evaluation is performed after each modification/infection.

The traditional genetic algorithms and bacterial evolutionary algorithms can be preferably extended with a local searching phase (e.g. Levenberg-Marquardt, see above) between the recombination and mutation operators which enhances the convergence of the optimization. These methods are called memetic algorithms. By combining a stochastic optimization with a local searching method, global optimums can be found with sufficiently good precision.

Although active obstacle avoidance is not performed by the robot platform (it can be performed easily by built-in ROS tools) it is checked whether a potential radiation position is closer to an obstacle in the 3D map than a predefined minimum distance (that can be set as an input parameter). It is also possible that a position which could theoretically be a radiation position, cannot be physically reached by the robot (e.g. because of its dimensions and distances between furnitures or other objects in a scene). These regions can also be discarded based on an input information when a radiation plan (candidate) is built up.

However, the fact that one radiation position cannot be approached by the robot does not necessarily mean that the whole individual should be discarded, since the remaining positions might be worth preserving. Still, it has to be taken into consideration that these individuals do not represent disinfection plans which can be executed. Therefore, the fitness values of these individuals are multiplied by a ratio. This ratio is computed by dividing the number of accessible radiation positions by the required number of radiation positions (in other words, the ratio of those radiation positions is determined which can be “realized” compared to all of radiation positions).

Since the 3D models might contain hundreds of thousands of voxels, the irradiance estimation is usually time-consuming. However, to accelerate the process, individuals may be evaluated in parallel. Moreover, the irradiance values that correspond to a specific lamp position and radiation time can be stored to avoid recalculation. The irradiated 3D models may be identifiable according to unique keys, and if one of the position-time pairs appears in an individual, the previously computed irradiance values can be loaded. The crossover, mutation and selection steps are cyclically performed, until the predefined maximum number of generations is reached (selections can be performed in the crossover and the mutation operators as well).

Based on the testing results, the proposed algorithm would be able to improve the quality of disinfection, however, its real impact cannot be easily determined, since it is hard to decide which voxels can be reached at all by the UV-C rays. For instance, the theoretical best Pᵢ value might be 0.5 or even 0.9 depending on the structure of the room. The number of radiation positions also has a huge impact on the efficiency (see the various embodiments for this parameter). Furthermore, if the elements were distinguished according to their surface type, the optimization might lead to even better results.

In the optimization process the evaluation of the individuals may be time-consuming due to the large number of voxels. To tackle this problem, the irradiance values that correspond to different lamp positions are stored so that recalculation is not required.

The evaluation often takes a significant amount of time, however, it has to be noted that if the area to be disinfected does not change much over time, the optimization should only be performed once. Otherwise, the former best individuals can be used as the initial population of the new optimization process (learning may be based on the—at the beginning—human-interacted path recommendation and geometric irradiation simulation). It can be a good utilization of the invention that the necessary irradiation procedure may be learnt based on the earlier paths for the new obstacles.

Another stochastic method to optimize the path of the disinfection robot could build that trajectory (i.e. the set of radiation positions; the path is also meant as such a structure) incrementally (see below described in details in connection with FIGS. 10A-10B). In some words, such an incremental path generation is based on the following. Starting from a given position in the 3D model the program could generate a set of new positions where the robot might move. The irradiance values are estimated for those positions and a subset of them is preserved which contains the best positions. This method is continued until a pre-determined number of positions is reached or until a sufficiently good disinfection result is obtained.

In FIG. 10A the incremental path generation method is illustrated as another stochastic solution (see below for detailed description).

According to the approach of incremental path generation, the radiation (disinfection) plans are encoded the same way as in evolutionary algorithms, i.e. a set of radiation positions and times but in this case these plans are built incrementally.

In this approach first a set of initial disinfection plans is generated which contain one radiation position and time and then new positions are added until a sufficiently good disinfection plan is found. In every iteration each disinfection plan is extended in multiple ways. More precisely, for each disinfection plan multiple new positions and radiation times are generated in each iteration thus the number of possible solutions will increase (see also operational steps S192a, S192b). This process is also illustrated in FIG. 10B.

At the end of each iteration the disinfection plans are evaluated by the irradiance evaluation method the same way as in the genetic algorithms, and the best candidates are preserved while bad solutions are discarded (cf. operational steps S194 and S195).

The output of the incremental path generation algorithm is a fully built disinfection plan that can contain any number of radiation positions, however, a limit may be defined for the number of positions and the overall disinfection time as well (for the details of the algorithm, see below).

As the possible solutions in the genetic algorithms are crossed and mutated it may happen that two or more individuals will be very similar. For instance, if only a few genes (e.g. one or two) of an already good solution are modified in an iteration and an even better solution is obtained this way, it is quite likely that both individuals survive. In this case there would be two very similar but still different individuals. Consequently, it may happen that two individuals will only differ in radiation times but the 2D radiation positions will be the same.

The case is very similar when incremental path generation is used, in the sense that there are multiple solutions (equivalent to individuals in genetic algorithms; in this case multiple incrementally built radiation plans) that contain the same 2D radiation positions but different radiation times belong to them. On the one hand, when the best solutions are selected in an iteration, it may happen that a few of them belong to the same spatial plan but different radiation times appear. On the other hand, in each iteration for instance n new positions and for each new position k new radiation times are generated. The difference between this approach and genetic algorithms in the sense of time is that in this case the new times are generated randomly in each iteration just like the 2D positions, while in genetic algorithms those are evolved from the initial population.

FIG. 10B gives an illustrative insight to the concept of incremental path generation (in other name—according to the fact that there are discrete radiation position(s) in a radiation plan —: incremental plan generation) showing fully exemplary incremental build of radiation plans. In this case it is also true that the radiation positions—constituting radiation data pairs with corresponding radiation times—and it is only illustrative that these discrete points are connected to each other. The interconnections forms arrows which show the direction (order) of the incremental generation. Similar grounds are to be applicable to the name of the algorithm itself (regarding the word, path). However, a path can be built in a sense that a good (economic) order of the radiation positions (order of visiting) can be defined based how these have been built.

For the illustrative insight in FIG. 10B several furnitures 202a-202e (not all of them are designated by a reference number) are arranged in a scene 200 realized as a room.

In FIG. 10B, the black filled circles denote possible robot positions (radiation positions 204, 206a-206d, 208a-208f, 210a-210d) and possible disinfection plans are connected by arrows. To make the figure easier to understand, only one start radiation position 204 is displayed in the bottom right corner of FIG. 10B next to the door.

Solid, dashed and dotted arrows denote the added positions in the first, second and third iterations respectively (these levels are also reflected in the reference numbers of the radiation positions). As can be seen some disinfection plans contain less than four positions, these plans were discarded at the end of a previous iteration (e.g. those ended in radiation positions 208a, 208b, 208c and 208f, i.e. these have not been continued).

The disinfection plans can be further extended by new positions to obtain a sufficiently good solution but fourth and subsequent iterations are not shown in FIG. 10B to make the illustration clearer.

Note that to each radiation position, one or more radiation times can be assigned. This way, each plotted disinfection plan may correspond to several virtual disinfection plans that contain the same radiation positions but with different radiation times.

At the end of each iteration the best available disinfection plans are preserved (cf. operational step S195 in FIG. 10A) and those that failed to produce acceptable results are discarded (not used in further iterations). The selection can be performed similarly as in genetic algorithms when for instance the possible parents are selected.

Before the selection of the best solutions, all candidates are evaluated (cf. operational step S194). Then three different methods can be used to select the best ones (truncation, roulette, tournament selection).

The roulette wheel selection (i.e. fitness proportionate selection) first normalizes the fitness values so that their sum is equal to one, then the possible solutions are sorted in descending order by their fitness values, thus to each of them belongs an interval between 0 and 1 which is proportional to its fitness value. After that, several (it depends on how many solutions should be preserved in each iteration) random numbers are generated between 0 and 1 and after checking which interval the random numbers fall within, the corresponding solutions are preserved. When a solution has been selected to survive, the fitness scores are normalized again before the next random number is generated.

If tournament selection is applied, then subsets of all possible solutions are selected randomly (the size of the sets, i.e. the number of elements in a set can be defined as an input parameter) and the individuals with the highest fitness values in each set are preserved.

Let us assume as an example that there are thirty radiation plan candidates and ten has to be preserved. The groups are formed totally randomly. On the one hand, ten groups can be created with randomly selecting their elements, so that each element belongs to only one group. Alternatively, the groups may be created so that an element can belong to more than one group. In either case, the number of groups, for which the elements are randomly selected has to be equal to the number of radiation plans to be preserved. Then the best individual in each group is selected to be preserved. If an individual is selected to more than one groups, and it is the best in all of them, then this individual would have ‘clones’ in the selected set which can be discarded (deleting duplicates because one of them is enough), in this case, new groups are generated one-by-one with randomly selected candidates until the required number of different individuals are selected (e.g. ten).

In an embodiment connected to FIGS. 10A-10B (but somewhat more general from that is outlined in FIGS. 10A-10B, detailed as an operational step S190 of incremental path generation) the first radiation plan generator module is based on—is established/realized by means of—an incremental path generation algorithm, and, after generating the one or more plan candidate in the plan candidate generating step (cf. operation step S191, wherein it is given that a set of random positions and—corresponding radiation—times are generated for the mobile robot each of which represent a disinfection (radiation) plan; ‘for the mobile robot’ is also not generally required in the hereby outlined embodiment), in course of an iteration cycle (cf. operational step S147, which is represented by an arrow coming back from the decision step to the starting step of the iteration cycle) of a third irradiation iteration
cloned plan candidates are generated for each plan candidate of the one or more plan candidates (cf. operational step S192a, wherein it is given that each disinfection plan is cloned several times),
a plurality of extended plan candidates are generated by extending each of the cloned plan candidates with an additional radiation data pair (cf. operational step S192b, wherein it is given that for each clone a new radiation position is generated; naturally, also a respective radiation time is generated; it is noted that in FIG. 10A this is formulated in a slightly way, as in operational step S193 these extended disinfection plans are cloned again and for each new radiation position different radiation times are generated, i.e. there may be clones in which only the radiation times of the new extension position are different, this meant also under the framework of the presently detailed embodiment),
a respective irradiance ratio estimation is generated in the irradiation evaluation step for each of the plurality of extended plan candidates (cf. operational step S194, wherein it is specified that all disinfection plans are evaluated according to the irradiation evaluation method),
checking a fulfilment of a first condition of the radiation plan sufficiency criterion for the plurality of extended plan candidates (cf. operational step S196, wherein it is investigated whether a sufficiently good disinfection plan has been found or not), wherein
   in case the first condition is fulfilled (‘yes’ branch of the investigation in operational step S146a), the first radiation plan is obtained in the radiation plan choosing step based on the plurality of the extended plan candidates (in this case the radiation plan choosing step is performed similarly to other embodiments but on extended plan candidates; also cf. operational step S197, wherein it is given that an optimized disinfection plan is obtained which contains one or more radiation positions and the corresponding radiation times),
   in case
      the first condition is not fulfilled (‘no’ branch of the investigation in operational step S146b) and,
      when the first condition is not fulfilled for at least the second time, a second auxiliary proportion of
         an actual highest efficiency proportion of the irradiance ratio estimation and the total radiation time of the plurality of extended plan candidates and
         the previous second efficiency proportion parameter is above a second predetermined development parameter,
   the highest efficiency proportion of the irradiance ratio estimation and the total radiation time of the plurality of extended plan candidates is given to a previous second efficiency proportion parameter and the one or more plan candidates for a next iteration cycle is constituted by at least a part of the plurality of extended plan candidates (cf. operational step S147 going back to operational step of S192a for cloning), wherein a total radiation time is obtained for each plan candidate by summing up the corresponding one or more radiation time for all of the one or more radiation position,
   in case
      the first condition is not fulfilled (also ‘no’ branch of the investigation but with different additional condition below, see operational step S144a) and,
      when the first condition is not fulfilled for at least the second time, the second auxiliary proportion of
         an actual highest efficiency proportion of the irradiance ratio estimation and the total radiation time of the plurality of extended plan candidates and the previous second efficiency proportion parameter is below the second predetermined development parameter,
   the first radiation plan is obtained as the extended plan candidate having the highest efficiency proportion of the irradiance ratio estimation and the total radiation time.

It is a general note that constructions for subtypes of plan candidates like extended plan candidate could be also called as first, second, etc. (type, kind) plan candidates.

In a nutshell, in the above embodiment, the options have a structure somewhat similar to the embodiment connected to FIG. 7B:
if the first condition is fulfilled, thus there is an acceptable plan candidate, the (final) radiation plan is obtained in the radiation plan choosing step, according to the above detailed principles, but in this case, it is selected from the plurality of extended plan candidates,
if the first condition is not fulfilled then the incremental path generation process continues with new cloning and extensions in case when there is sufficient development compared to the previous level of the process (controlled by a second predetermined development parameter); this is controlled by the help of the second auxiliary proportion (cf. with the first auxiliary proportion), the previous value of which is stored appropriately;
if the first condition is not fulfilled but there is not enough development compared to the previous cycle, the iteration is terminated and the (final) radiation plan is obtained based on the efficiency proportion.

Reinforcement learning is a further, somewhat similar approach for implementing the optimization (i.e. the radiation plan generator module). In this approach, at any given position the program may determine which other positions the robot can move into. These radiation positions are evaluated and rewards (prizes) are assigned to them. During planning the robot selects its next position according to a parameter which determines how likely it is that the robot will go to the best position (see below for details in connection with FIGS. 11A-11B).

The machine learning in connection with reinforcement learning is basically the learning of the optimal path (radiation plan). In learning algorithms usually weights are modified to obtain ideal results. The modification of these weights is the learning. In the case reinforcement learning, the learning is the phase where rewards are assigned to the radiation position-time pairs (i.e. exploration). During the exploitation phase the weights are not further modified thus this part is not considered as learning. However, both the learning phase (i.e. exploration) and the selection of the optimal disinfection plan considering the previously computed rewards (i.e. exploitation) are considered as artificial intelligence methods.

During the exploration phase the value of this parameter is lower thus the robot prefers to explore its environment (cf. operational step S224), while during the exploitation phase (cf. operational step S225) this value is higher therefore it is more likely that the robot will go to a position that provides the best disinfection result.

In FIG. 11A a flowchart of the steps of reinforcement learning are given. To use reinforcement learning, a large set of possible radiation positions and times are generated inside the target environment.

During the learning phase (phase for learning, where are the good positions; exploration), the robot virtually moves between these positions and tries to find an optimal disinfection plan. The movement of the robot may be limited so as it cannot move to positions that are too close or too far from its current location. Before the robot selects its new location, a set of possible new positions are considered and to each of them a prize is assigned. This prize is calculated by the irradiance evaluation method and may reflect how good a position is by itself or as part of the current disinfection plan. This way multiple prizes can be assigned to the same radiation position—radiation time pair according to how it was reached by the robot.

Reinforcement learning usually begins with an exploration phase when the robot has a higher chance not to select the best possible position as its next location (cf. operational step S224). While the algorithm progresses with the learning it reaches the exploitation phase (cf. operational step S225) in which the robot prefers to select one of the best available positions to extend the disinfection plan.

The output of the reinforcement learning algorithm is the same as for stochastic methods (i.e. a radiation (disinfection) plan) but there is less focus on randomness. In reinforcement learning, when the possible positions are generated in the beginning, a set of possible radiation times are assigned to each of them. Those might be fixed radiation times for all of them (e.g. 3-5-10 minutes, or more generally, radiation times between 1 and 15 minutes, preferably between 2 and 12 minutes, particularly preferably between 3 and 10 minutes; these intervals—also by exchanged interval limits—are applicable for a value of the radiation time for a radiation position in all embodiments; these radiation time limits may be affected by the power of the light source; light source is considered to be the most general term, a light source may comprise e.g. more lamps) or can be randomly generated separately.

The same scene 200 (room) is utilized for illustration of reinforced learning in FIG. 11B. Let us assume that circles solid (solid circles 232) and dotted (dotted circles 234) both denote possible radiation positions generated for the reinforcement learning. To each such position several radiation times belong, for instance 1, 2, . . . 10 mins.

During planning, the construction of the radiation plan is so that the robot virtually starts in a given position and for which the irradiance distribution map is computed. Then it selects a subset of the other positions and times (or all of them) and computes the fused irradiance distribution maps for them assuming that the first position is already known. After the evaluation the robot selects an advantageous new position—time pair (radiation data pair) and continues the process until a sufficiently good solution is found. This process can be executed parallelly starting from different positions.

In FIG. 11B, dotted circles 234 denote those positions that belong to an optimal disinfection plan, and the numbers written in them is the corresponding radiation time (2, 4, 2, 5, 4 and 5 minutes from right to the left).

The prize of a radiation data pair may be determined based on how well they increased the disinfection level for each disinfection plan when they were added to it. The optimization begins with the generation of a large set of positions and corresponding radiation times which can be identical for every position or different (cf. operational step S221). The network of these 2D positions and times can be thought of as a 3D graph in which an optimized solution has to be found. In the first part of the optimization several starting positions are selected. Then new positions-times are added to each such disinfection plan after an evaluation step. The algorithm examines several possible new position-time pairs based on some criteria (for instance in the proximity of the previously selected one but not too far from it) and selects one of them as the addition of the disinfection plan.

In the beginning of the process (i.e. exploration) not necessarily the best position-time pairs are added. During the exploration phase the algorithm computes the prize for several possible new radiation positions but usually not the best one is selected. The main purpose of this process is to reach all possible radiation position—radiation time pairs, preferably in multiple radiation plan candidates and assign prizes to them. If the algorithm always selected the best options, then there would presumably be areas in the environment that could not be explored. As the algorithm can only select the new radiation position inside a distance range, it may happen that a set of good radiation positions is cordoned off by a set of less suitable radiation positions. Consequently, to complete the exploration task properly, usually not the best available radiation position will be selected as the next addition to the radiation plan until the prizes have not been assigned.

When a position-time pair is considered for addition, it is computed how this new element in the disinfection plan improves its fitness score. A single position-time pair can be considered by multiple disinfection plans thus different improvements can be computed for it. The prize of each position-time pair is computed according to these improvements. However, the prize can also be inversely proportional to the radiation time, since for instance if the same position provides similar improvement in three minutes and five minutes, then the former should have a higher prize.

The first (exploration) phase of the optimization is therefore used to obtain prizes for all or at least most of the position-time pairs (cf. operational step S224). Since these position-time pairs are examined as the part of several disinfection plans, the prizes will reflect not just the independent benefit of them but also how well they might contribute to a possible solution consisting of multiple positions and radiation times.

Consequently, this computation method provides more information compared to the case when all radiation position-time pairs are evaluated only independently. When the exploration process (phase) is finished, many of the position-time pairs have a prize assigned to them. By selecting the ones with higher prize, the space of possible candidate position-time pairs can be reduced. It may be required that for assigning a prize a radiation data pair is taken into account (“touched”) for at least two times (e.g. 2-5 times). When enough touching is reached then the prize is obtained by e.g. a simple average.

In the second phase (i.e. exploitation; cf. operational step S225), the algorithm only considers those candidates which have higher prize and seeks to find an optimal solution. For instance, let us assume that there are twenty position-time pairs which are represented as a graph. Then we would like to find a subgraph in it which may consist of one or more nodes and it has to provide either a sufficiently good irradiance level or the disinfection has to be performed under a time limit depending on which optimization criteria is required.

In the following, an embodiment being in connection with FIGS. 11A-11B (however somewhat more general than FIGS. 11A-11B) is defined, wherein the first radiation plan generator module is based on a reinforcement learning algorithm.

In course of the method in this embodiment
in a radiation data pair generating step a plurality of radiation data pairs are generated (this is a new step, where not plan candidates but radiation data pairs are generated, cf. operational step S221, wherein it is specified that a large set of positions are generated inside the disinfection area—i.e. generally in the scene in relation with the 3D model—along with corresponding radiation times),
in a starting radiation data pair assignment step after the radiation data pair generating step (in this step starting radiation data pairs are generated with prizes, i.e. starting radiation positions along with respective radiation times)
   the one or more plan candidate is generated in the radiation plan generating step (generation of the one or more plan candidate is realized here) by selecting one or more radiation data pair as respective one or more starting radiation data pair of a plan candidate,
   a respective irradiance ratio estimation in the irradiation evaluation step are generated for the one or more starting radiation data pair (irradiation evaluation is realized here) and a prize parameter value is assigned to each of the one or more starting radiation data pair based on the irradiance ratio estimation (in FIG. 11A a different and specified illustrating type approach is applied for illustration, thus operational steps S222 and S223 are also being in connection with assigning, and also with assigning of the exploration step; operational step S222 is termed as the mobile robot virtually moves—i.e. in the approach of FIG. 11A this is formulated as a virtual movement, in the present embodiment this is not as specified—between these positions and it assigns different prizes to the positions based on the irradiance estimation method and how it reached that position; operational step S223 is termed as the robot can only move to a subset of positions that fall in a given distance region from the current position),
in an exploration step after the starting radiation data pair assignment step until a prize parameter value is assigned to each of the plurality of radiation data pairs (i.e. it is the goal to assign prizes to the radiation data pairs, cf. operational step S224 being formulated more specifically: in the beginning—i.e. in the exploration step/phase—the robot prefers exploration thus it has a bigger chance of selecting less advantageous positions), in course of an iteration cycle of a fourth irradiation iteration
   one or more exploration plan candidate is generated by extending a corresponding previous plan candidate having a previous irradiance ratio estimation (these coming from the starting radiation data pair assignment step) with a respective additional radiation data pair,
   a respective exploration irradiance ratio estimation is generated in the irradiation evaluation step for each of the one or more exploration plan candidate, and
   a prize (goodness) parameter value is assigned to each additional radiation data pair based on the difference between the exploration irradiance ratio estimation of the corresponding exploration plan candidate and the previous irradiance ratio estimation of the previous plan candidate, and
in an exploitation step after the exploration step (cf. operational step S225 being formulated more specifically: as the learning process progresses the robot starts to prefer those positions that have the highest prize)
   one or more exploitation plan candidate is generated from the radiation data pairs based on the respective prize parameter values assigned thereto (the prize parameter can help to generate advantageous plan candidates),
   a respective exploitation irradiance ratio estimation is generated in the irradiation evaluation step for each of the one or more exploitation plan candidate, and
in the radiation plan choosing step after the exploitation step the first radiation plan is obtained (cf. operational step S226, wherein, formulated in the approach of FIG. 11A, it is given that the robot finds the most advantageous disinfection plan which contains one or more radiation positions and the corresponding radiation times) based on the exploitation plan candidate having
   the highest exploitation irradiance ratio estimation, or
   the lowest total radiation time, wherein total radiation time is obtained for each plan candidate by summing up the corresponding one or more radiation time for all of the one or more radiation position, or
   the highest efficiency proportion of the irradiance ratio estimation and the total radiation time.

In connection with the radiation plan choosing step of the reinforcement learning, the followings are noted. It is given above that any of the above parameters (irradiance ratio estimation, total radiation, efficiency proportion) may be used for selecting the (final) radiation plan. To this aspect, the followings are taken into account.

It is possible to use a radiation time limit or an irradiance limit in a similar way as detailed above, but it is also possible not to specify a limit.

In case the radiation time limit is used than, if there is at least one among the exploitation plan candidates that fulfils the condition of the radiation time limit, then the (final) radiation plan is selected based on the irradiance ratio estimation (that, which has the highest of this value).

In case the irradiation limit is used than, if there is at least one among the exploitation plan candidates that fulfils the condition of the irradiation limit, then the (final) radiation plan is selected based on the total radiation time (that, which has the lowest of this value).

If any of the limits is used, but the limits are not fulfilled or no limit is utilized, then the (final) radiation plan is selected based on the efficiency proportion (that, which has the highest value). Furthermore, when a limit is expected to be reached in the reinforcement learning approach, then if there is no sufficiently good radiation plan (in view of the limit) after the exploitation step, then the exploitation step can be repeated by generating new plan candidates. This repeating can be done maximum for a reinforcement maximum iteration number, when this number is reached, the (final) radiation plan regardless of the limit (i.e. based on efficiency proportion) is selected.

Some embodiments of the invention relate to a method for generating a radiation plan of a scene (cf. FIGS. 12A-12B) in particular for a disinfection robot adapted for radiating disinfection light, which embodiments have a second radiation plan generator module based on deep learning, in the course of which
providing or generating a 2D or 3D scene representation of a second scene (cf. scene 200 of FIG. 12B), and
generating (cf. operational step S245 in FIG. 12A) a radiation plan for the 2D or 3D scene representation by means of a second radiation plan generator module based on deep learning trained (i.e. the neural network of the deep learning algorithm is trained; cf. operational step S244) by a plurality of first radiation plans of respective first scenes generated (cf. operational step S242) by the above detailed embodiments of the method according to the invention (based on evolutionary algorithm, incremental path generation or reinforcement learning).

The above mentioned 2D or 3D representation will be available for the respective radiation plan in the training phase, i.e. the radiation plan utilized for the training is determined vis-a-vis the 2D or 3D representation naturally in order that the second radiation plan generator module become familiar to that representation which will be used as an input (this representation is typically a 3D model, from which a top view/floorplan—2D representation—may be easily generated; the 3D model will be available since it is needed for performing the optimization with the other methods).

Furthermore, some embodiments relate to a system for generating a radiation plan of a scene in particular for a disinfection robot adapted for radiating disinfection light (being equivalent as the method of the previous two sections, i.e. based on deep learning). The system comprising a second radiation plan generator module adapted for generating a radiation plan for a 2D or 3D scene representation of a second scene and based on deep learning trained by a plurality of first radiation plans of respective first scenes generated by the above detailed embodiments of the method according to the invention.

The optimization of the disinfection plan can also be performed using trainable—preferably by means of backpropagation or by other training methods—machine learning algorithms (e.g. Deep Neural Network). The method and system for generating radiation plan being on this basis use the 3D model of the environment as their input while the characteristics of the UV-C lamp and the factors that affect irradiance estimation are considered as parameters of the network. In other words, the radiation plan generator module of this method and system needs only 3D model to be radiated (disinfected) as an input and it does not directly use the irradiation evaluation module applied in the above introduced other embodiments.

A—preferably deep—neural network applied in this framework may contain, among others, partly or fully connected convolutional, deconvolutional, recurrent, pooling, normalization and dropout layers. The output of such a network would be the set of ideal one or more radiation position and corresponding radiation time. The network can be for instance trained by backpropagation in order to obtain optimal weights. One of the great advantages of these networks is that after training they can be applied to newly reconstructed environments immediately without requiring to perform the step of optimization using directly the irradiation evaluation module. In this case, the applied neural network represents the irradiance estimation and can obtain an optimal solution, however, the exact irradiance distribution still to be computed for the ideal one or more radiation position, if needed.

Another solution is to create an optimized disinfection plan based on images (local sensor data) as inputs (this is a 2D representation as mentioned above under 2D or 3D representation). The network could learn which image types correspond to good disinfection results and consequently when the training is finished the disinfection plan can be obtained according to only images (one type of learning is to predict optimal path based on camera images).

In FIG. 12A operational steps of a method based on deep learning are illustrated.

To use a deep learning method in the optimization process (i.e. in determination of the radiation plan, in this approach the optimization is built into the neural network trained by deep learning) effectively, a large set of previously computed optimal disinfection plans is required to use in the training data.

The optimal disinfection plans can be obtained by applying one of the other embodiments of the method according to the invention (all of those introduced above before the present embodiment, i.e. those based e.g. evolutionary algorithms, incremental path generation or reinforcement learning, thus those which use the irradiance evaluation directly) in real-world scenarios, i.e. for example in a lot of different indoor environments that require disinfection.

However, since all of the algorithms can be used in a synthetic environment, it may be more convenient to create—even randomly—a large set of various virtual worlds like in the embodiment of the method illustrated in FIG. 12A (cf. this step with operational step S241, wherein a large set of—many times complex to facilitate training—virtual environments is generated).

The 3D reconstruction, irradiance estimation and optimization tasks can be performed in these worlds and optimized disinfection plans can be obtained (cf. operational step S242, wherein for each virtual environment an optimal disinfection plan is obtained using either evolutionary algorithms, incremental path generation or reinforcement learning).

The plans then need to be encoded as 2D/3D models which contain information about the radiation positions, times and the quality of disinfection (cf. operational step S243, wherein the optimal disinfection plans which contain the radiation positions, radiation times and the irradiated 3D model—this latter is optional as highlighted above—are encoded as 2D/3D models; inputting quality of disinfection is also optional, basically only the 2D/3D models and the optimal radiation plan is given as an input).

If the computations are carried out for a sufficiently large set of test environments and the results are fed into a neural network, it can be trained to find optimal disinfection plans for environments that it has not seen before (cf. operational step S244, wherein the large set of 2D/3D models are fed into a neural network which learns how to find optimal disinfection plan without directly using the irradiance estimation formula). In the case of deep learning, the irradiance evaluation method appears in the training phase but for the optimization the neural network does not directly use the irradiance estimation formula.

Finally, as given in operational step S245 in FIG. 12A, the neural network provides an optimal disinfection (radiation) plan.

Consequently, the deep learning approach does not work with irradiance values, but these can be obtained using the 3D model, the optimized disinfection plan and the irradiance estimation formula. A great advantage of deep learning is that the trained neural network can produce a disinfection plan which is equivalent to the ones provided by other optimization methods, but the evaluation of a large set of disinfection plans is not required for a new environment.

In the simplest case, this regression problem (with independent variable (3D model) and dependent variable (radiation plan)) can be tackled by a deep learning approach that would provide a convolutional neural network which works similarly to object detecting networks (e.g. YOLO—You Only Look Once, Alexey Bochkovskiy et al.: YOLOv4: Optimal Speed and Accuracy of Object Detection, arXiv:2004.10934v1). The input can be the whole 3D model or a top view image of it (or other 2D or 3D representation which fits to the input the handling of which is to be learned by the neural network of the deep learning approach; naturally, it is typically a 3D model) and the network tries to find optimal 3D/2D positions in the input model along with corresponding radiation times.

This could work similarly to YOLO as it returns a set of bounding boxes (with centre coordinates, height, width and label) but this time the output would be a set of radiation positions (with coordinates and radiation times). The neural network produces results with a certain quality which has to be compared with optimal radiation positions which were computed with any of the optimization methods (evolutionary algorithms, incremental path generation, reinforcement learning). By comparing the outputs of the convolutional neural network with the optimal solutions it can be trained to provide optimal solutions on its own.

The irradiance estimation formula in this case appears as part of the feedback (encoded in the optimal disinfection plan(s), i.e. can be obtained based on it).

Another solution within the deep learning approach would be to represent the task as a graph-optimization problem (to utilize candidates based on Convolutional Graph Neural Network). Then the whole 3D environment would be covered by a large set of radiation positions which would be the nodes. The task is to find a minimum set of these nodes that are still able to provide sufficiently good coverage and disinfection. The nodes could be connected by edges that are associated with weights which represent how good disinfection can be achieved using the connected nodes. The radiation times would also appear as weights in this configuration.

In FIG. 12B the same scene 200 (room) is illustrated as in FIGS. 10B and 11B. In this case the deep learning based approach is illustrated in the room. This is an example top view image of the area to be disinfected.

Solid circles 236 and black filled circles 237 are part of the current solution provided by the convolutional neural network. Dotted circles 238 denote the optimal disinfection plan which was computed with any of the optimization methods. Black filled circles 237 mean that those radiation positions are not required. On the one hand, the spatial distance should be as low as possible between the annotated (dotted; ground truth) and regressed positions (solid and black), and on the other hand, it is expected from the neural network to regress positions having the minimum number.

The arrows between solid circles 236 and dotted circles 238 denote the spatial difference between the predicted and optimal radiation positions. Note that although radiation times are not marked in the figure they are still present and belong to the radiation positions. The difference for them can be computed similarly to the spatial difference. Both the spatial and the time difference can then be used as errors which are fed back to the neural network that is trained this way. Accordingly, FIG. 12B illustrates the training procedure of the deep learning based approach.

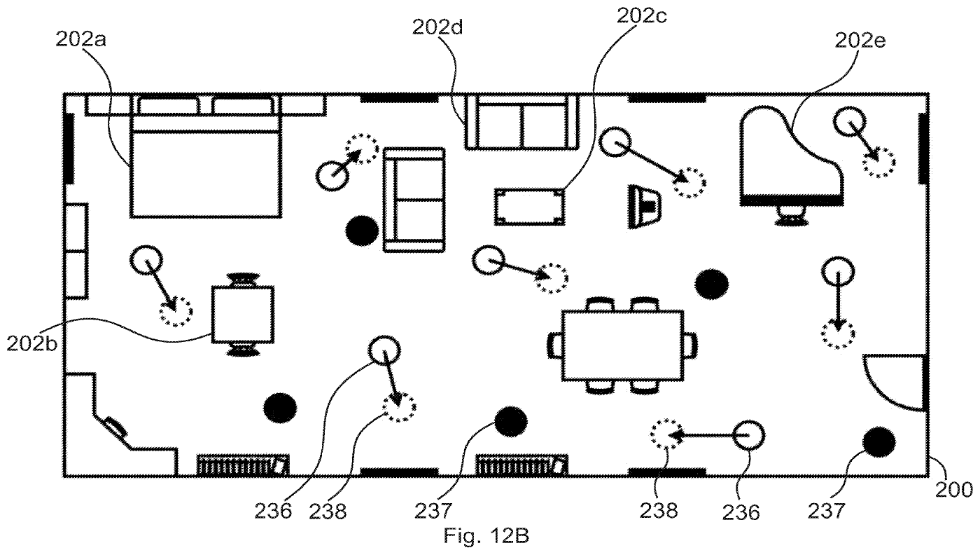

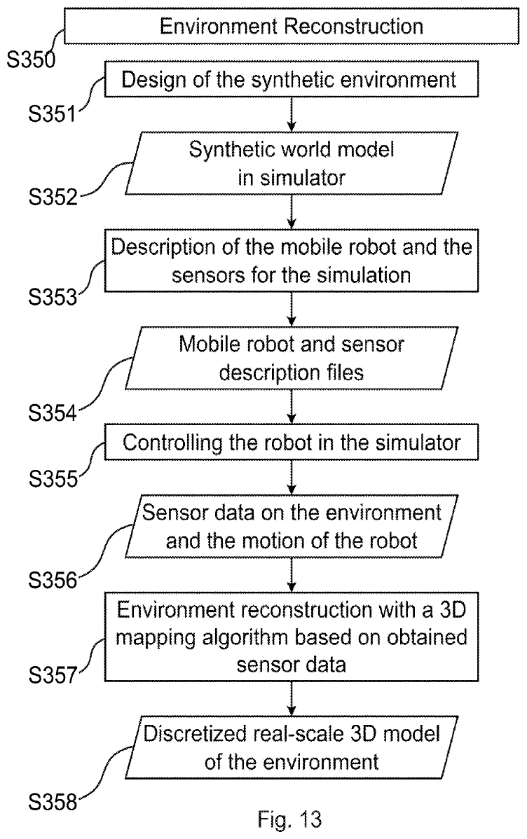

An embodiment of environment reconstruction—shown as operational step S350—is illustrated in FIG. 13. In order to train a convolutional neural network, a large training dataset is required which contains optimized disinfection plans (see above). The optimization disinfection plans can be obtained by the described optimization methods but a large set of test environments is still required.

Naturally, the whole optimization and disinfection process can be performed in real-world environments. However, it may be more convenient to use virtual test environments since they can be easily modified and by placing various objects in different configurations in rooms with complex shapes, a sufficiently diverse training dataset can be obtained (cf. operational step S351: synthetic world model in simulator).

Regardless of the type of the environment (i.e. synthetic or real-world) the 3D mapping process can be performed similarly. The only difference is that virtual mobile robots and sensors have to be used. An appropriate software for creating virtual test environments is Gazebo (Nathan Koenig and Andrew Howard. Design and use paradigms for gazebo, an open-source multi-robot simulator. In 2004 IEEE/RSJ International Conference on Intelligent Robots and Systems (IROS)(IEEE Cat. No. 04CH37566), volume 3, pages 2149-2154. IEEE, 2004; cf. operational step S352: synthetic world model in simulator) and the obtained synthetic worlds can be explored by virtual robots and sensors.

The robot (e.g. Waffle, see above) and sensor (e.g. RGB-D camera) description files are provided by dedicated packages of the Robot Operating System (ROS; cf. operational step S353: description of the mobile robot and the sensors for the simulation; in operational step S354 mobile robot and sensor description files are received). ROS can also be used to control the virtual robot (cf. operational step S355: controlling the robot in the simulator) and acquire sensor data, although it might be worth noting that this process is carried out the same way in real-world environments.

The 3D mapping is also performed similarly, the virtual robot acquires virtual sensor data about the virtual world using for instance a SLAM algorithm (e.g. RTAB-Map; cf. operational step S356: sensor data on the environment and the motion of the robot) and builds a discretized real-scale 3D map (cf. operational step S357: environment reconstruction with a 3D mapping algorithm based on obtained sensor data) which can be then used to compute an optimal disinfection plan (cf. operational step S358: discretized real-scale 3D model of the environment is obtained).

It has to be noted that in some cases the original virtual environment may be directly converted to an appropriate 3D representation (e.g. OctoMap). However, it is more realistic to accomplish this task by 3D mapping, for instance, those areas will not be mapped which could not be accessed by a real robot.

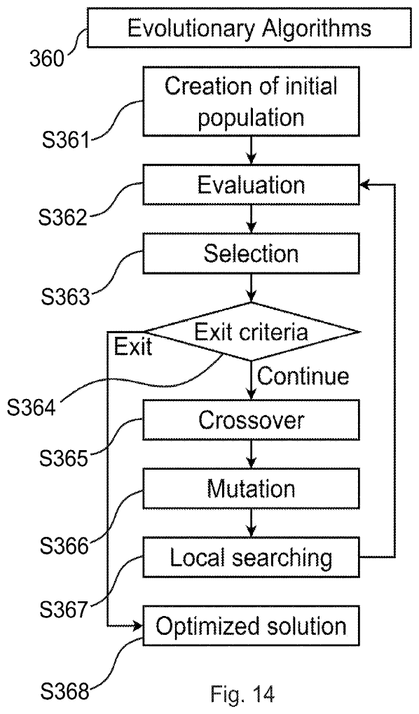

FIG. 14 shows a so-called optimization block (radiation plan generator). This block is very similar to FIG. 7A where a more generalized explanation of evolutionary algorithms is presented. However, the present FIG. 14 gives a more schematic outline of the process of generating a radiation plan (cf. operational step S368 labelled as optimized solution), which is specific to the concept based on genetic algorithm.

The order of operational steps is as follows:
in operational step S361: (random) creation of the initial population (individuals encode the coordinates of light source pose and radiation times);
in operational step S362: evaluation (performing irradiance estimation and computing fitness scores for the results);
in operational step S363: selection (based on the order of individuals obtained through evaluation);
in operational step S364: decision (on whether a sufficiently good solution has been found), in case of exit, the optimized solution is reached in operational step S368;
in case of continuation of the process:
   in operational step S365: crossover (obtaining new solution through recombination);
   in operational step S366 mutation (obtaining new solutions through independent modification)
   in operational step S367 local searching in memetic algorithms and feedback to evaluation (operational step S362), which is the first step of the next iteration.

In the following irradiance/exposure monitoring (with other term in the description, visualisation), i.e. continuous updating of the 3D model with the continuously built up irradiation is described.

In the dynamic simulations the time factor is also taken into consideration and the colour of the voxels is updated constantly. Every step consists of two phases.

First, the new colour of the voxels is calculated from the distance, angle and time values measured in the previous step. After that the new values are computed w.r.t. (with regard to) the current state and are stored connected to the corresponding voxels. The outputs of the algorithm are a voxel or a point cloud representation (it can be also ‘and’ instead of ‘or’, the points of the point cloud may be the centre of the voxels) of the environment (most generally, 3D element representation), painted according to the estimated irradiance. The static and dynamic simulation results are described in the following.

We tested the simulation software in our office with both of the depth cameras shown in FIG. 22 (Xbox 360 Kinect and Intel RealSense d435i depth cameras). The images shown in connection with this were obtained from the simulation executed with Intel RealSense d435i depth camera. In the visualisations the colour values of the voxels correspond to the estimated irradiance. If the colours applied in the irradiation figures are not the shades of grey, but e.g. purple, the higher the red and blue channel values (being contributions to the purple colour; set to be the same value) are the higher the estimated irradiance is while (a close to) maximum value would mean that a proper disinfection has been achieved.

The proposed simulation (software) can be used with either a standing or a moving camera. On the one hand the simulation (software) is capable of creating static simulation where the colour values of the voxels are set relatively according to the considered distance and angle factors. On the other hand a dynamic simulation can also be executed where the colours represent the estimated irradiance over time. The results of the static and dynamic simulations are illustrated in FIGS. 20A-20E, 24A-24C and in FIG. 19A-19H, 25A-25D respectively (see the description of these figures below).

When the optimization process ends, the radiation plan (disinfection plan, individual) with the highest fitness score (generally, irradiance ratio estimation) is selected. At this point the irradiation level can be preferably visualized by the help of the 3D mode. In this case the 3D model of the room (generally, scene or environment) is coloured according to the corresponding irradiance values. The simulation results can be visualized either statically (it is always a final type result) or dynamically (on the one hand, appearing of radiation positions one after the other, on the other hand, increasing of the irradiation value as a function of time, or both).

In the optimization step, the irradiance values are computed based on the total radiation time at each position. Similarly, if the static mode is selected, the final results of the irradiation process can be displayed. It is rather useful, when the impact of different factors needs to be visualized, which can be seen in FIGS. 20B-20E, 24A-24C. Static mode can also be used to examine how the manual adjustment of radiation positions affect the distribution of irradiance.

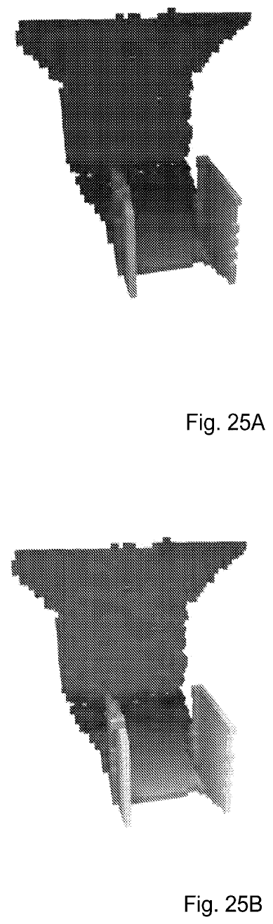

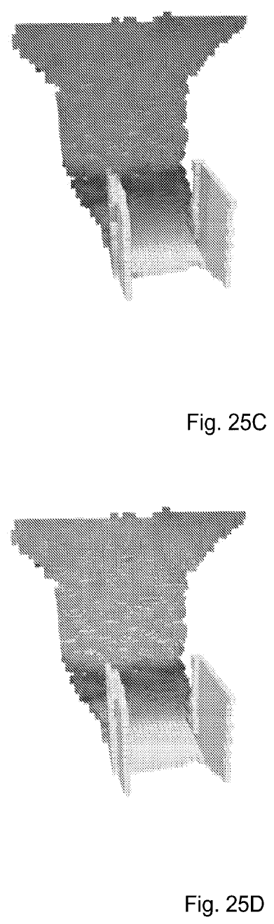

In FIGS. 24A-24C impact of different factors on irradiance is illustrated on the scene part illustrated in FIGS. 23A and 23B on RGB and depth images provided by the RealSense camera. In FIGS. 24A-24C (as well as in FIGS. 25A-25D) those voxels (small cubes) of the scene part can be observed which are detectable by the depth camera from the point of view illustrated in FIGS. 23A-23B. The location of the depth camera can be somehow determined based on the views; this is noted also for interpreting the distance below in connection with e.g. FIGS. 24A and 24C.

FIG. 24A shows the impact of distance from the light source. FIG. 24B shows the impact of angle of incidence. As well as, FIG. 24C shows the combined impact of distance from the light source and angle of incidence.

Accordingly, FIG. 24A shows that the closer voxels receive greater irradiance dose regardless of the angle of incidence, and, since distance is the only factor in this case, the irradiation decreases by the distance (for interpretation of the distance, see above).

FIG. 24B shows an opposite phenomenon, as the farther voxels can receive greater dose if the angle of incidence is smaller (i.e. close to zero). To the surface in the back the irradiance falls approximately perpendicular (for the definition of angle of incidence, see FIG. 18); these voxels have a light grey colour. As well as the irradiance rays are approximately parallel to the two closer walls, accordingly, these cannot receive much irradiance (shown by black voxels).

Furthermore, FIG. 24C—where both the factors of distance and angle of incidence are taken into account—shows that the distance (inverse square law) is more significant than the angle of incidence (cosine law); see the shades of the colour of the voxels: the voxels being in the foreground are the lightest.

In contrast, the dynamic mode highlights the irradiation process as illustrated in FIGS. 25A-25D (distance and angle of incidence are taken into account). The total radiation time is divided into shorter periods and the different states of the irradiated model are stored. Then, it can be monitored—even visually in the exemplary FIGS. 25A-25D—how the number of properly irradiated surface elements increases as the time goes by.

The dynamic simulation of irradiation illustrated in FIGS. 25A-25D is computed from both the distance and angle of incidence over time. These figures show that dynamic simulation is not limited to the display of the final results of multiple radiation positions. The different stages of the irradiation process from a single position can also be visualized at different times during the disinfection. This method helps the user to understand how well the room has been disinfected for instance after 10 minutes.

If FIGS. 25A-25D are considered one after the other, we can observe that in FIG. 25A all of the voxels are relatively dark, but the closer voxels are a little bit less dark. In FIG. 25B essentially all of the voxels become lighter, especially the closer voxels which were already lighter in FIG. 25A. This tendency continues in FIG. 25C, where the irradiation of the closer voxels is quite acceptable. Furthermore, in FIG. 25D, the surface in the back also become even lighter.

In FIGS. 24A-25D shades are shown, i.e. the how much the irradiance is proceeded in the respective voxels (in FIGS. 24A-24C this means a resultant irradiance value of the voxels, as well as in FIGS. 25A-25D the irradiance values are depicted as a function of time, accordingly, the irradiance mainly increases from figure to figure).

It is noted with emphasis that the illustration of the evolution of the irradiance is to be differentiated from that whether a given voxel reached an irradiation limit or not.

Reaching of the limit can be illustrated in a binary form (with two possible colours for each voxel: limit reached or not). However, reaching of the limit can be visualized also in evolution like figures. Thus, if a voxel reached close to the lightest colour (lightest grey) in the visualization approach of FIGS. 24A-25D then its irradiance is above the irradiance limit.

The created system was tested in several synthetic environments that were created in Gazebo during the development process. First, the different factors that affect the irradiance estimation were examined, i.e., the value for each voxel was computed by considering only one parameter at a time. The static simulation results are illustrated in FIGS. 20B-20E, 24A-24C. For all simulations, the higher these values—i.e. the voxel becomes lighter—the higher the computed irradiance.

Accordingly, FIGS. 19B-19H illustrates the different stages of the dynamic simulation, as new optimized radiation positions are added. The simulation can be divided into any number of stages, and for each of them, the corresponding irradiance values are computed separately. The current irradiation state of the displayed model is then constantly updated as the simulation progresses. For the simulations visualized in FIGS. 19B-19H, all aforementioned factors were taken into account. As FIGS. 19B-19H show, the lamp positions are evenly distributed and essentially all regions are affected by the UV-C light. To further improve the performance of the system, the number of radiation positions can be/is handled in respective embodiments (see above).

Thus, as shown in FIGS. 19B-19H in addition to the distribution of the irradiance on the surfaces of the 3D model (a 3D model 310 utilized also for FIGS. 19B-19H is shown in FIG. 19A), the optimized lamp positions can also be displayed (radiation positions 312a-312g). Furthermore, if reflected irradiance also has to be computed the incoming and reflected rays can be displayed (i.e. the rays themselves, not only the result of them on the irradiance), however it has to be noted that this function is rather time-consuming. Moreover, since the rays can only be visualized in the model if the corresponding voxels are marked as occupied, every ray has to be deleted right after it was cast in order not to affect further calculations.

In FIGS. 19A-19F the 3D occupancy grid representation of the primary test environment for the UV-C disinfection robot is illustrated. The mapping—leading to 3D model shown in FIG. 19A—was performed using an RGB-D camera and RTAB-Map. FIG. 19A shows the details of the test environment (equipment in the room) which may not be easy to recognize due to the colours which are set according to the irradiance distribution. There are minor missing regions in the map which follows from the structure of the robot that performed the 3D mapping (e.g. around the bottom left corner in FIG. 19A, which was hidden by other objects when the 3D model was generated). The RGB-D camera was placed at approximately to a height of 141 mm and it could only reconstruct those areas that were directly “visible” from its viewpoint.

In FIGS. 19B-19H building up a radiation plan from seven radiation positions is illustrated. FIGS. 19B-19H illustrate different stages of the radiation plan generation (with considering only direct irradiance), as new radiation positions are added. For each of the stages, the corresponding irradiance values are computed separately (see also Table 1 below). The current irradiation state of the displayed model is then constantly updated as the stages progress.

As FIGS. 19B-19H show, the lamp (radiation) positions are—more or less, especially without the seventh radiation position—evenly distributed and there are only a few regions which were not affected by the UV-C light. To further improve the performance of the system, the number of radiation positions can also be optimized by the algorithm. For the first view, there is only a small difference between FIGS. 19G and 19H, but a further radiation position 312g is added to FIG. 19H.

In Table 1 below the results for an indoor test environment are shown.

TABLE 1

| Number of positions | x | y | Radiation | Fitness |
|---|---|---|---|---|
| (ref. numeral) | coordinate | coordinate | time | score |
| 1 (312a) | −10.24 | 2.68 | 1000 | 0.145871 |
| 2 (312b) | −3.71 | 2.83 | 1000 | 0.301017 |
| 3 (312c) | 2.38 | 2.81 | 1000 | 0.469519 |
| 4 (312d) | 3.75 | −2.67 | 1200 | 0.634525 |
| 5 (312e) | −2.43 | −1.75 | 1200 | 0.704950 |
| 6 (312f) | −10.91 | −1.56 | 1000 | 0.814980 |
| 7 (312g) | −6.31 | −0.37 | 800 | 0.832627 |

In Table 1 the columns from left to right represent the number of radiation positions (along with their reference numeral in FIGS. 19B-19H) in a current radiation plan 312, the x and y coordinates of the radiation positions (x-axis goes in the direction parallel with the longer of the 3D model and increases to the left, while y-axis is perpendicular to it being parallel to shorter side of the 3D model and increases downward, see FIG. 19B), the corresponding radiation times and the obtained fitness scores (irradiation ratio estimations), respectively.

The unit of the values of x and y coordinates is meter. In this case, the radiation times are values without units and only the ratios of values are important. Since the radiation times and the power of the light source are non-real values in this simulation, the radiation limit above which a surface element is considered as sufficiently well disinfected will also be a theoretical number.

However, the aim of Table 1 is to highlight the fact that if well distributed radiation positions are incrementally involved in a disinfection plan, then the overall result of irradiation significantly improves by the addition of each new position (see the evolution of the fitness score in the last column of Table 1). Consequently, by setting different time parameters (different order of magnitude), light source power and radiation limits, different results could be obtained but the trend would be similar.

As it was mentioned earlier, in order to correctly represent the area to be disinfected at least two-voxel-wide surfaces are required. To achieve this, several new elements were inserted in the 3D model, the majority of which intentionally cannot be reached by rays cast from any position inside the room. These elements usually belong to the external faces of the walls (vis-à-vis the radiation positions of the lamp). To compensate for this effect, the fitness scores in this table were obtained by dividing the number of sufficiently well irradiated elements in the 3D model by the number of elements in the original 3D model (i.e. before expansion to two-voxel wide walls).

Now we turn FIGS. 20A-20E, wherein FIG. 20A illustrates a 3D model which is the same as the 3D model 310 of FIG. 19A but all the way around (to fit to the plots of FIGS. 20B-20E).

In FIGS. 20B-20E the impact of four irradiance parameters that were considered. Namely, characteristics of the UV-C lamp/light source in FIG. 20B, visibility of the voxels in FIG. 20C, distance from the centre of the light source in FIG. 20D, angle of incidence in FIG. 20E.

To highlight the impact of the characteristics, the lamp had to be placed close to a wall as can be seen in FIG. 20B, where it is taken to radiation position 320. For this section of the wall, as the angle between the beam and the horizontal plane increases, the irradiance value decreases which is denoted by dark voxels (see the parabolas on the wall; under the lamp, the irradiation is low because of the high angle to the horizontal plane). The impact is much less significant for surfaces that are further from the lamp, since in this case, the aforementioned angle is relatively small. However, it has to be noted, that although the computed colours are proportional to the irradiance, its distribution is not as uniform as the image suggests.

In FIG. 20C the lamp is placed approximately in the centre of the 3D model 310 into a radiation position 325. In FIG. 20C, the colours of the voxels reflect the number of rays that reached them, i.e., the value of the visibility parameter (see also FIGS. 15A-15C, those voxels are white in FIG. 20C which are reached by all of the bundles, and there are more level greys for those voxels which are reached by smaller number of bundles). It is an important factor since it determines whether a surface element is irradiated or not. As it can be seen, those areas which are covered by an object (e.g. a table or a section of the wall) were reached by less rays thus their colours are darker.

Moreover, FIG. 20C highlights the importance of using at least two voxel wide surfaces. In contrast to some other figures, the two sides of the walls are differently coloured, since the outer side cannot be irradiated if the lamp is placed inside the room.

In FIG. 20D the lamp is also placed approximately in the centre of the 3D model 310 into radiation position 325. FIG. 20D demonstrates how the distance between the voxel and the centre of the light source affects the estimated irradiance. According to the inverse square law, the irradiance is inversely proportional to this distance, thus it changes rapidly, as the colours of the voxels suggest. There is a saturation inside the circle around the radiation position 325, within 8 times lamp height the irradiation is estimated this way, the decay (transition) is observable at the periphery of the circle and the irradiation goes down within short distance (the colouring becomes black).

Although this is a very dominant factor, it has to be noted that the FIG. 20D only illustrates relative values, i.e., they are not directly related to the irradiance which is computed using the Irradiance Estimation Formula. However, based on the simulation results, it could be assumed that the irradiance on the surface elements that are further than 10 m from the light source is negligibly small thus they do not need to be considered during the calculations.

In FIG. 20E the lamp is also placed approximately in the centre of the 3D model 310. Lastly, FIG. 20E shows the impact of the angle between the incoming beam and the local surface normal vector, i.e, the angle of incidence.

As it can be seen in FIG. 20E, the irradiance is maximal on surfaces that are perpendicular to the UV-C ray cast from the light source, regardless of the distance between. In contrast, on surfaces that are parallel to the horizontal plane, the values are much smaller due to the orientation of the lamp (its longitudinal axis is parallel to the vertical direction), which are denoted by darker colours. Next to the lamp itself the impact of the lamp is intensive within a circle (somehow below the lamp; we do not count with the body of the robot, however it is easy make it to the part of the model, an ideal lamp characteristic is taken into account).

Thus among others, an optimization procedure based on the above ray-tracing methodology by optimizing the movement of the robot along its path based on the data obtained from previous operation by using learning methods, like Deep Learning trained by previous paths, control instructions and searching plans.

To create a realistic simulation algorithm (software), several challenging tasks have to be accomplished. About the proposed simulation (software), see the followings. As detailed above the simulation of the irradiation process consists of several steps including the reconstruction of the environment, the irradiance estimation on the surrounding surfaces and the visualisation of the results. As the application—realized in the invention—was created for autonomous robots the Robot Operating System (ROS) can be preferably decided to use as the framework of the software (M. Quigley et al.: “Ros: an open-source robot operating system” in ICRA WS on Open Source Software, 2009.).

For using the framework of the invention, the characteristics of the lamp is preferably to be determined, i.e., how irradiance varies on a sphere around the light source. Moreover, as shown by the illustration of building the 3D model, according to an option, the global environment of the robot needs to be reconstructed in order to visualise the surrounding objects as the robot is moving on its trajectory.

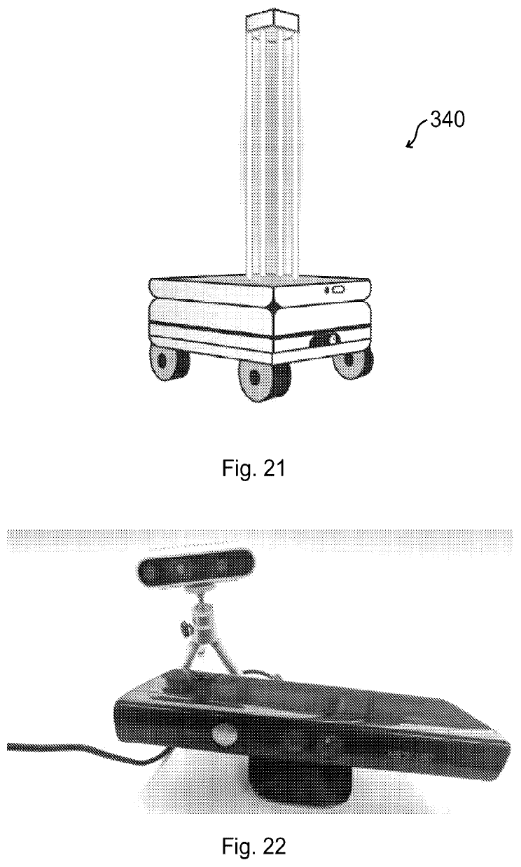

The ideal 3D representation preserves as many details of the environment as possible and allows for sufficiently fast updates for online operation. By creating a 3D map, the whole irradiation process can be simulated before the disinfection begins. The model also allows for considering the structure of the environment. An expressive reconstruction can be achieved by using an RGBD camera which creates a 3D model which fulfils the previously mentioned criteria. During the development process we used an Xbox 360 Kinect and an Intel RealSense d435i depth camera which can be seen in FIG. 22 along with the sketch of an autonomous UV-C disinfection robot in FIG. 21 and a pair of RGB and depth images in FIGS. 23A and 23B, respectively.

The irradiance on the reconstructed surfaces can be computed during the movement of the robot or after the operation. In either case those parts of the global model have to be selected which are currently affected by the UV-C light based on the position of the robot. Depending on the architecture of the robot and the placement of the tubes, different kinds of models can be applied to accomplish this task.

After the currently affected parts of the model are determined, the irradiance can be estimated. The overall efficiency of the irradiation process depends on the exact characteristics of the UV-C light source. However, in an example we only considered those fundamental factors which can be easily determined and have a significant impact on the calculation. These are the distance from the light source, the angle of incidence and the time of irradiation. Here, an algorithm for irradiance estimation is proposed which takes into consideration both the characteristics of the environment and the UV-C tube.

In many cases the UV-C lamp is placed in the centre of the room and does not move while performing the disinfection. However, it can be advantageous to select multiple lamp positions as the structure of the room might be complex. Therefore, the created algorithm has to be able to compute optimized locations (radiation positions) where the mobile robot should stop to radiate. An optimization method is also provided to compute appropriate radiation locations inside the rooms, i.e. those positions where the mobile robot should stop to radiate for a certain period of time (cf. these with the above details of the invention given above).

To visualise the impact of these factors, we set the colour of the model elements according to the estimated irradiance relative to a previously defined maximum value. The proposed simulation (software) and the test results are described in the following sections (cf. FIGS. 24A-25D, by the help of which and other figures, a visualization tool is presented with which the effects of various parameters and the process of radiation can be demonstrated).

The construction of a new disinfection robot is out of the scope here, as it was more important to create a method that only depends on the sensor configuration. Consequently, the algorithm was tested in a synthetic environment using a virtual robot (see above), and the model of a real UV-C lamp. Although the effectiveness of the disinfection can be determined in a practical way (i.e. directly measuring how the number of bacteria or viruses decreases over time), the proposed algorithm relies on sensor measurements and the characteristics of the lamp.

It has to be noted that the factors of temperature, humidity, obstacle avoidance and motion detection were not detailed in this description.

In the following, measurement of UV-C lamp characteristics is illustrated. In order to properly estimate irradiance, the characteristics of the UV-C lamp need to be determined (for this a lamp model can be used for the irradiance estimation as an input, see the description of FIG. 18, in particular the characteristics parameter, I(α(x))). On the one hand, the lamp can be considered as a point light source. Then, in the simplest case, it can be assumed that the lamp radiates equally in all directions, thus no measurement would be required. On the other hand, the lamp can also be modelled as a cylindrical light source, however, in this case the computation is more complicated.

A lamp that was used in the experiments was relatively small compared to those normally mounted on disinfection robots. If the irradiated surface is sufficiently far from the lamp, then it can be modelled as a point light source, however, the distribution of the radiation cannot be considered as uniform. Consequently, in such a case it should be measured how the lamp radiated in different directions.

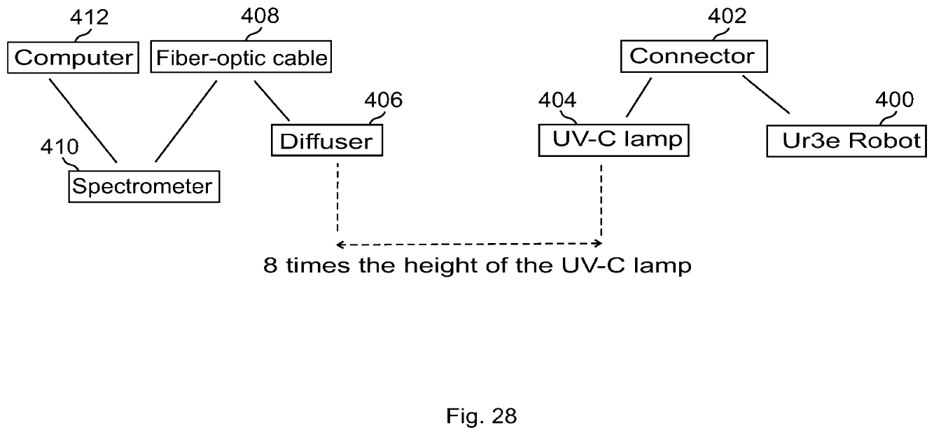

FIG. 28 illustrates an exemplary measurement setup. In this example, a UV-C lamp 404 was moved by a UR3e Robot 400 (which is a robotic arm; https://www.universal-robots.com/products/ur3-robot), as its position needed to be precisely set. To attach the lamp to the end of the robot arm, a new connector device 402 was designed.

The radiation was measured by a spectrometer 410 and the signals were transmitted to the sensor through a diffuser 406 and a fiber-optic cable 408. The spectrometer 410 was connected to a computer 412 and the measured data was processed using AvaSoft (https://www.avantes.com/products/software/avasoft-full-avasoft-all).

In order to consider the UV-C lamp 404 as a point light source, it had to be placed sufficiently far from the diffuser 406. In the experiments this distance was approximately eight times the maximum diameter of the lamp (i.e. its height).

First, the centre of the lamp had to be defined as the reference point, then the required positions could be set using the controller of the robot arm. To determine the distribution of radiation on a spherical surface around the lamp, it had to be rotated around two perpendicular axes, one of which was its longitudinal axis. Based on the structure (more precisely the symmetry) of the lamp one or more angular positions (in the experiments four angular positions evenly on the interval between 0° and +45° (in general between 0° and 360°)) have to be selected around the longitudinal axis of the light source. Similarly, several angular positions (in the experiments fifteen angular positions evenly on the interval between −70° and +70° (in general between −90° and +90°)) have to be selected around another axis which is perpendicular to the longitudinal axis (which will hereafter be referred to as “x axis”). Furthermore, the pixel sensitivity of the sensor was not known, therefore absolute irradiance could not be measured. However, the measurements could be compared in a small wavelength range, between different angular positions, thus relative values could be obtained which were suitable to characterize the lamp.

Disinfection efficiency of the applied UV-C lamp is possibly needed to be more exactly determined. On the one hand, the distribution of absolute irradiance can be measured around the light source with a suitable sensor. On the other hand, it might be a more practical solution to directly measure how the number of viruses and bacteria decreases over time due to the effect of the UV-C light.

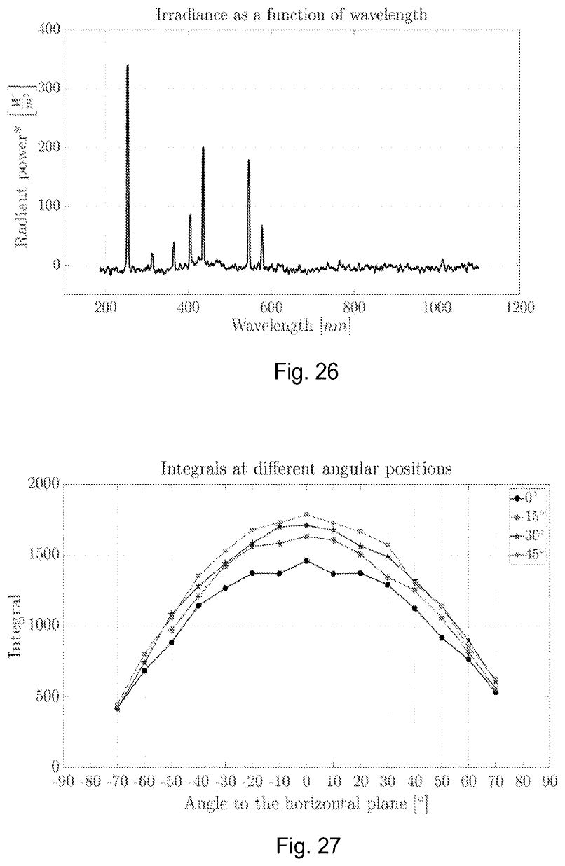

In the example, the characteristics were measured relative to a dark reference, thus it had to be set before the lamp was turned on. The spectrometer measured radiant power per small wavelength range (approximately 0.6 nm) as can be seen in FIG. 26. This chart shows that the lamp emitted both visible and UV light, however, for this application, the important peak is the one around 254 nm. To estimate a quantity that is proportional to irradiance, the area under this peak had to be calculated for each angular position of the lamp. First, the relevant interval around the maximum of the peak had to be found, then the area could be calculated using the trapezoidal rule.

The results are illustrated in FIG. 27. As it can be seen, each curve corresponds to one angular value around the longitudinal axis of the lamp, and the angular positions around the x axis are marked on the horizontal axis of the chart. Since there were no significant differences between the four curves, they were merged by computing the mean of the values, in order to simplify further calculations. This simplification is also worthwhile, because the angular position of the lamp around its longitudinal axis, relative to the robot, depends on how it connects to the socket. If the lamp was removed between operations, its angular position might change, which cannot be easily taken into consideration in the irradiance estimation.

The resulting curve was normalized so that its maximum value was one. It can be seen in FIG. 27, that the higher the absolute value of the angle around the x axis (i.e. the angle to the horizontal plane), the lower the radiant intensity. Consequently, if only the lamp characteristics was considered, the irradiance would be the greatest on surfaces at the same height as the centre of the lamp.

Accordingly, in FIGS. 26-27 measurement results of the characteristics of the UV-C lamp are shown. In FIG. 26 the measured radiant power is illustrated as a function of wavelength in a specific angular position of the lamp. The * next to radiant power means that these are only relative values. FIG. 27 shows the value of the area under the peak around 254 nm for every angular position.

The lamp characteristics was taken into account during irradiance computation as an angle-dependent parameter which is denoted by I(α(x)). The α(x) angle is illustrated in FIG. 18 (it is the angle between a plane being perpendicular to the longitudinal axis of the lamp and a beam 301, i.e. a ray), where x denotes the vector pointing from the centre of the light source to a specific surface element. If a(x) did not correspond to any of the fifteen angular positions of FIG. 27, then I(α(x)) was computed using linear interpolation. Finally, it has to be noted that this light source model can preferably be used for relatively small lamps, because it was assumed that the lamp is further from most of the irradiated surfaces than eight (5-10) times its height.

The robot preferably learns semantic interpretation of the usual scenes. This meant on that during the reconstruction process the robot may recognize—as a result of the semantic interpretation—various objects in the environment, for instance tables etc.

The recognition of objects may enhance the 3D reconstruction process, since the robot knows that some parts of the environment still need to be explored if incomplete objects are present. Object detection also helps to decide which obstacles can or cannot be removed by the robot. For instance, a table is too heavy to be removed but small objects should not necessarily be considered as obstacles. This way the movement of the robot can be optimized—even by removing small objects (the 3D model may change during the application of the radiation plan); in these cases the radiation plan may be recalculated if needed—and it can generate simpler paths between radiation positions.

Finally, if various objects are recognized, the robot can give an estimate of what material the objects might be made of (e.g. a table is usually made of wood) and thus the reflection parameters can be estimated.

The semantic interpretation helps it to predict (currently occluded) object's depths (size in the depth direction), normals, extension, etc. In this way on the one hand the SLAM based navigation can be enhanced. On the other hand, considering the measured and predicted values (ray-tracing, distances, etc.) which are influencing the performance of the irradiation, this performance can be maximized as the robot can select the optimal position to start the disinfection.

In the following, robust usage based on dynamic SLAM procedure is described. The disinfection system can be pushed to any (starting) position, at any time, switching on/off randomly, continuing the process from a new starting position, because it is incompatible with the daily routine in e.g. a hospital, and it can work in the free (of disturbing objects and humans) time periods. In this case, a SLAM algorithm is working continuously, mapping the working area and the related changes, and finding the actual position from the actual views' evaluation. This procedure solves the problem of robust working in cooperation with the usual business of that environment: the SLAM system continuously learns an environment and localizes the robot position at any time; moreover, it stores into the SLAM map the 3D irradiance dose-map. So, it can be robustly fitted into the environment and calculating the irradiance dose and positions optimized to the automatic SLAM tracking.

The irradiation process can be performed by more than one disinfection robot, especially if a larger indoor environment needs to be cleaned. In order to execute this task efficiently, it is important for the robots to communicate with each other, for instance, share the acquired 3D models of the environment and the current disinfection level on the surfaces. This problem can be tackled by multi-agent SLAM, where the mobile robots communicate with each other and share information. They can localize themselves relative to each other and the environment while creating local 3D maps and a shared global 3D map as well.

In this case, multiple robots can be used during the reconstruction process and during the disinfection process as well. The disinfection can be performed during or after the reconstruction process.

It can be important to use multiple mobile robots when either the area is very large or very complex and/or the disinfection has to be performed in a short time. Then the whole disinfection plan (i.e. the radiation positions and times, which have been determined by the help of the method according to the invention) can be divided into subplans each of which is carried out by one mobile robot. For instance, it may happen that a subset of regions are accessible from one direction while the others are accessible from another direction, then it may be convenient to divide the disinfection plan.

Another example for preferred use of multiple robots is when a territory can only be disinfected for short separate times. Then multiple mobile robots can move to the optimized radiation positions and rapidly turn on and off according to whether it is safe to radiate or not. In this case the movement between radiation positions might have taken too much time.

Another example is when a mobile robot malfunctions, then another robot may continue the disinfection plan from the current state.

Finally, since the mobile robots are able to divide disinfection plans and share the 3D models along with the current disinfection level, advantageously, a robot can easily recognize and consider changes in the environment that affects the subplan and consequently the operation of the other disinfection robots.

US 2021/0089040 A1 is mainly based on cleaning, thus does not disclose the main parts of the invention. At least parallel radiation position and radiation time optimization, as well as irradiance evaluation is not disclosed in this prior art document.

Some further conclusions are also taken about the invention highlighting some advantages of the invention or of the respective embodiments.

It is a main advantage of the invention that the radiation plan is generated based on handling both radiation positions and radiation times by means of an artificial intelligence approach by the help of which the optimal radiation positions and radiation times can be found using irradiance evaluation to control the irradiance on a 3D element-level (e.g. voxel-level). Thanks to this, it is controlled on a high level on what level a 3D scene is irradiated and, at the same time, the irradiation is produced with a high effectiveness, since the radiation plan is generated so that the irradiation to be as efficient as possible (optimal) for that specific scene for which it was built.

It is relevant to note that according to the invention not the visibility of the parts of the 3D model is checked but it is controlled how much the 3D model of the scene is irradiated and reaching of an irradiation level is checked on 3D element-level. It is noted that by the help of direct ray-tracing, the visibility itself can also be checked, but the visibility would not give information of the sufficient irradiation without checking as it is done in the invention. Moreover, preferably in the invention, direct ray-tracing is only one factor which is taken to account when the irradiation is estimated.

Accordingly, the invention advantageously increases disinfection control to a high level in contrast with prior art approaches where the disinfection is done without checking that the scene is satisfactorily disinfected. In the invention, irradiation for the whole radiation plan—built from a set of one or more radiation position and radiation time pairs—is checked. Moreover, such irradiation checks of radiation plan candidates leaded to the final chosen radiation plan. Controlling also the radiation time—i.e. it plays the role of a variable —, not only the radiation position, is also a key element of the invention in order that the irradiance level is controlled.

It is also a relevant feature of the invention that the radiation plan generated by the method and system according to the invention comprises one or more radiation position and respective one or more radiation times. In other words, the radiation plan is built up from one or more discrete (separate) radiation position in each of which one or more radiation position the radiation lasts for the respective radiation time corresponding to that radiation position.

The radiation positions are discrete, i.e. (if there are more) separated from each other. In other words, the radiation positions does not constitute a continuous trajectory, i.e. in the invention the radiation (disinfection) does not performed continuously along a route, but it is performed in discrete radiation positions. This is substantiated by the fact that a radiation time (by other term, a radiation time period) is assigned to all radiation position. Accordingly, the radiation only performed in the selected radiation positions (points), the duration information is only taken into account for the radiation positions.

It can be also said to a single radiation position that it is discrete; this is the case if the method according to the invention results in that a single radiation position (with a respective radiation time) is enough for sufficient irradiation. This may be the results in case of radiating (disinfecting) a simple shaped (rectangle based) room. It is noted that, independent from the result, the specific operational steps of the method according to the invention have to be performed. Thus, the invention can be clearly defined even if only one radiation position-radiation time is resulted.

Thus, the robot (or the radiation effecting device, which can be realized also by other ways) radiates only (exclusively) in these points (i.e. in the one or more radiation position), and does not radiate on the distance between the radiation positions, i.e. on the route when a next radiation position is approached. However, it is not preferred to switch off the radiation source between the radiation positions (i.e. on its route), since it can give a (relatively small, but) additional contribution to the irradiation, and, furthermore, it can be disadvantageous to switch of on the route because the radiation source e.g. may need time for reaching the operational temperature. However, according to the invention, we do not preferably count by this contribution, but preferably only with the contribution radiated from the one or more radiation position.

It is noted in this respect that the method and system according to the invention are suitable for generating the radiation plan, i.e. to have as a result a set of one or more radiation data pair of a radiation position and a radiation time. Thus, the radiation plan may be called as a discrete radiation plan, a radiation plan being discrete/discretized in space (meaning the separation of one or more radiation position), or a radiation plan with discrete one or more radiation position. To summarize, according to the invention, not a trajectory is determined by the help of the optimizer.

It is, furthermore, noted also that checking of sufficient level of irradiation would be a task very high computational demand for radiation performed continuously along a trajectory (in our view it could be only a very slightly substantiated estimate or prediction, not an elaborated one, applied according to the invention). According to the invention, it has been chosen to radiate in discrete position(s) in which case the level of irradiation can be—i.e. possible to be—checked according to the approach detailed in this description. From some aspect a kind of “double checking”/“back-checking” of irradiation is performed according to the invention.

Moreover, with radiating from fixed positions the sufficient (amount of) irradiation is more realistic to be reached: typically, according to the invention, the radiation time is in the order of minutes (e.g. a radiation time can be selected to be between 1 and 10 minutes, see also above for possible radiation time values), while with continuous movement the radiation is much more distributed and not concentrated (the accumulation of the irradiation is of different kind), it seems harder to control whether an irradiation limit is reached or not. In case of a trajectory with continuous radiating the velocity of processing along the trajectory would be adjustable.

It is, furthermore, noted that it is a secondary aspect from the point of view of the invention that exactly what is that effecting the radiation at that position for the duration of the radiation time (the power of the radiation, as well as radiation characteristic, and other parameters constitute input data, as detailed elsewhere).

The radiation position may be shifted to a predetermined height; accordingly, the position means a 3D coordinate, from which the height coordinate may be the same for all radiation positions (can be a part of the parameters of the input data). However, also the height coordinate can be handled as a variable.

It is also noted although the total radiation time can be preferably optimized according to the invention, this aspect of optimization is not done in itself but in the context of controlled irradiation checking by the help of the irradiance evaluation. This takes the system of variables and control points according to the invention to a complex level, where by the help of the artificial intelligence based radiation plan generation the space and time variables for radiation are handled parallelly.

To summarize, roughly speaking it can be said that characteristic in the invention that an artificial intelligence based generation of radiation plan candidates (pronouncedly with space and time variable) is performed and the radiation plan candidates are checked by the help of irradiance evaluation. The final radiation plan is obtained by using preferably iteratively these two operational steps.

In the following, some—sometimes less relevant—aspects of the various embodiments of the invention are summarized.

Firstly, we turn to the environment reconstruction. The precise estimation of irradiance highly depends on the position of the preferably UV-C light source relative to the irradiated surfaces. Therefore, the 3D model of the area—i.e. a scene—to be disinfected has to be created. On the one hand, it is important to preserve as many details of the objects in the area as possible to precisely estimate irradiance on their surfaces. On the other hand, as the time required for the calculation highly depends on the size of the 3D map, it must not contain too many elements.

The reconstruction process can be performed using various mobile platforms and sensor configurations. An appropriate 3D model can be obtained by several sensors including RGB camera, depth camera, Lidar etc. These sensors can be carried by a human or any mobile platform that is capable of moving in complex environments. If carried by a robot, it might move along a predetermined trajectory or navigate by itself using a localization method. In these cases the robot also needs to detect and avoid obstacles.

As the sensors are carried around in the room, its 3D model can be created using various 3D reconstruction methods which are dependent on the sensor configuration. For offline reconstruction, a Structure from Motion pipeline can be used, while a Simultaneous Localization and Mapping (SLAM) algorithm might be able to perform online mapping.

Based on the applied reconstruction method, various 3D representations can be obtained which then can be used to estimate the UV-C irradiance precisely. The 3D space can be discretized in several ways, for instance, a traditional point cloud or voxel grid model can be used. In each case, the elements of the model can be extended with additional information (e.g., colour, occupancy, reflection parameter etc.). As obstacle avoidance is taken into account, the use of an occupancy grid (e.g. OctoMap, i.e. octree-based occupancy grid, see FIG. 19A) can be advantageous.

In addition to their locations in the 3D space and their occupancy values, other meaningful information associated with the elements of the 3D model can be stored. For instance, the colour values can be useful when the 3D model is visualized. The reflection and absorption parameters of the elements of the 3D model are also important since they significantly affect the irradiance estimation when indirect (reflected) irradiation is also taken into account. One way to determine these parameters in the model is to use learning algorithms (e.g. neural network, genetic learning) to distinguish different materials. These parameters can also be estimated by using an object detecting method combined with the obtained colour values. In both cases, the obtained parameters can be stored with the corresponding elements of the 3D model.

An initial 3D map of the room to be disinfected is required to estimate irradiance. However, it cannot be assumed that the environment remains unchanged, as the objects inside the room might be moved during and/or between operations. To handle this problem, the mapping algorithm should be able to monitor changes in the environment during the disinfection process and modify the 3D model in accordance with the obtained information. If the changes are significant in the 3D model, then the disinfection plan can be adjusted, and the modified disinfection plan can be considered and executed during the current or the next disinfection process.

Now, we turn to irradiance estimation. In order to precisely estimate irradiance values for the room, first those elements of the 3D model have to be selected which can be directly reached by the UV-C rays assuming that the UV-C light source is placed in a given position (cf. FIG. 20C). These elements in the 3D model can be selected in different ways.

One solution is to store the physical model related to the local sensor data that was recorded during the mapping process and compare it to the global 3D model. In this way, it can be determined which parts of the global model were reconstructed from a given position in the room and thus it can be assumed that these elements can be reached by the UV-C rays (this is an alternative for ray-tracing and computing visibility parameters; however, if indirect contributions are intended to be checked, then ray-tracing will be inevitably needed).

This challenging task can also be tackled by ray tracing. When this method is used, every element of the 3D model has to be targeted with one or more rays cast from the UV-C light source (cf. FIGS. 15A-C) and it has to be checked whether the ray(s) reached the element or not with sufficient dose (this latter is also taken into account in the Irradiation Estimation Formula).

The computational demand can be reduced by setting a maximum range for the UV-C light source, beyond which the elements of the 3D model are not taken into consideration. As the 3D space is in some way discretized, the 3D model cannot completely represent the real conditions of the room. Consequently, performing only one raycasting per 3D element might reduce the quality of irradiance estimation (a 3D element has three relevant surfaces, that is why more rays (i.e. a bundle) are needed from a point; also, it is preferred to have more than one starting point from a lamp, this helps to take into account that it has a finite size, i.t. it is not point-like).

Hence, although it increases the computational demand, it might be necessary to cast multiple rays from the UV-C light source. The source points from which the rays are cast can be selected evenly or randomly on the surface of the UV-C light source. The parameter which describes whether a 3D element can be reached by the UV-C rays from a given position of the UV-C light source can be considered as a logical value or a ratio. In the first case, an element can be reached or not, while in the second case, the number of rays that reached the target can be compared to the total number of rays that were cast in the direction of the element (see description of FIGS. 15A-15C also for these latter aspects).

The UV-C light source itself can be modelled in different ways, for instance, as a point light source or a cylindrical light source. In these cases, the radiant power is distributed on either a spherical or a cylindrical surface respectively (cf. description of FIG. 18, FIG. 20B). The distribution might be uniform, or it might be dependent on the direction. In the latter case, the exact characteristics of the UV-C light source has to be measured. Based on the type of the light source model, the dependency of irradiance on the distance from the light source can also be different. However, regardless of the type of the light source, the irradiance is in some way inversely proportional to the distance (cf. description of FIG. 17, FIG. 20D).

The irradiance is highest for those elements in the 3D model, for which the ray that was cast from the light source is perpendicular to the surface. However, the greater the angle of incidence (i.e. the angle between the ray and the local surface normal vector), the greater the area on which the radiant power is distributed and consequently the lower the irradiance, which is proportional to the cosine of the angle of incidence (cf. description of FIG. 18, FIG. 20E). Based on the applied 3D representation of the reconstructed environment, various methods (e.g. local plane fitting) can be used to estimate local surface normal vectors.

In addition to the above mentioned factors, the quality of the disinfection also depends on the time of irradiation (note—referring to the above—that the radiation time is a main factor for each of the radiation positions). Considering all the parameters and the characteristics of the lamp, the time required for proper disinfection can be calculated. Furthermore, if the duration of irradiation is set to a constant value, the accumulated irradiance for each element in the 3D model can be obtained.

The above mentioned parameters can be used to estimate direct irradiance on surfaces. However, since the local surface normal vectors are preferably computed, reflected irradiance can also be obtained. The reflected irradiance estimation is also affected by the previously mentioned reflection property of the surfaces which can be determined during the environment reconstruction process. Combining the direct and reflected irradiance computation, a more precise estimation can be performed which results in a high quality disinfection. Moreover, by leveraging reflected rays, those elements of the 3D model can be reached which otherwise could not be radiated due to obstacles. In addition to these parameters, other factors can also be taken into consideration such as temperature, humidity etc.

Now we turn to optimization, i.e. to the generation of radiation plan or one or more radiation plan candidate. The mobile platform that is equipped with the UV-C light source and various sensors can perform the irradiation in several ways. The simplest solution is to place the robot in the middle of the room and radiate the whole environment from that perspective. Although the computations are quite simple, this solution may not be advantageous because many parts of the environment can be covered by obstacles. Consequently, it would be advantageous to select multiple waypoints for the robot to go along them and potentially stop to radiate for a certain period of time in some designated positions. The number of these positions might be set manually or randomly, during the evolutionary algorithm, or determined using a learning algorithm.

These radiation positions can be preferably selected based on a learning algorithm or by a stochastic selection method. The stochastic selection method might be for instance, a genetic algorithm, a bacterial algorithm or a memetic algorithm, as well as incremental path generation. In either case, the potential solutions to the problem, for instance the ideal positions for radiation have to be encoded. In addition to these positions, the solutions might contain the corresponding radiation times. For each stochastic optimization method there is a fitness function which describes how good the potential solution is for the problem under study (the fitness function is in general the irradiance ratio estimation, so this statement can be generalized also for reinforcement learning approach). This fitness function might describe for instance the average irradiance value for the whole 3D model, or the number of elements in the 3D model for which the irradiance exceeded a predefined minimum value (this latter is the irradiance ratio estimation), or even the ratio of these elements can be used.

If a stochastic optimization method has been selected to evolve the potential solutions, first an initial set of such solutions need to be generated randomly (in e.g. evolutionary algorithms). In addition to the totally random generation, additional information about the environment might be included, for instance the dimensions of the room. Furthermore, if the room to be disinfected was radiated before, but for some reason (e.g. the room layout has changed) a recalculation is required, the previously optimized solutions can be used as the initial set to evolve. In other words, the previous results can be utilized, since the information is connected to discrete radiation position(s) which can be somehow “disconnected” and thus the radiation plan may be performed with interruptions or e.g. even by multiple disinfection robots (devices) as mentioned also above.

The potential solutions then can be iteratively combined and modified in order to obtain new and ideally better solutions. In e.g. genetic algorithms, the potential solutions can be combined through applying a crossover operator (e.g. one- or two-point crossover, uniform crossover etc., see FIG. 8). These crossover operators take two potential solutions and combine their attributes which results in two solutions that are different from the original ones.

The solutions for which the crossover operator is applied can be selected randomly or based on one or more criteria, for instance, the fitness function of the optimization method (this is such an application of fitness function, i.e. irradiance evaluation which can be applied on the top of its usage in selection of the final radiation plan). As the potential solutions have been evaluated according to the fitness function, the inputs (i.e. parents) of the crossover operator can be selected in several ways (e.g. roulette wheel selection, tournament selection etc., see above). After the crossover has been performed, different strategies can be applied to select which solutions should survive. One strategy is that the new solutions (i.e. offsprings) are preserved regardless of the computed fitness values. Another strategy is to select the two best solutions, but both the original and obtained solutions can be preserved.

Once the crossover operator has been applied, the selected outputs can be used as the input of the mutation operator which independently modifies the attributes of the solutions (cf. FIG. 9A). The mutation operator can be applied to a set of the potential solutions or to all of them. The modification of the attributes is usually associated with a probability distribution function (e.g. uniform distribution, normal distribution, etc.) (cf. also FIG. 9B). The mean of this probability distribution function is usually the original attribute in the potential solution while the deviation parameter can be generated randomly, optimized by a learning algorithm, or selected based on an initial assumption, for instance for the radiation positions, the dimensions of the room can be used.

The mutation, similarly to the crossover operator, is followed by an evaluation step. At the end of the iteration, either all potential solutions are preserved or an additional selection step can be performed in which a subset, usually the best several solutions (i.e. truncation selection), are selected to survive.

When instead of the genetic algorithm, a bacterial algorithm is used, then the applied operators are the bacterial mutation and the gene transfer. The former is responsible for the independent evolution, while the latter combines the pairs of potential solutions. In bacterial algorithms, although the evaluation is performed for every potential solution, separate selection steps are not included.

The memetic algorithms being also applicable extend the operators of genetic or bacterial algorithms with a local searching method (e.g. Levenberg-Marquardt, etc.), which might result in faster convergence thus less iterations are required.

Now, turn to the visualization, i.e. controlling of irradiance level throughout the 3D model. Once the irradiance estimation and the disinfection robot path optimization is finished the results can be visualized. In most cases, the original 3D model, obtained during the 3D reconstruction process, can be used as the basis for visualization. The most evident solution is to set the colour of the elements in the 3D model according to the estimated irradiance values. Moreover, the calculations can be carried out without taking all the aforementioned parameters into consideration at the same time, but the various factors can be visualized independently, like in FIGS. 20B-20E. This way the impact of the different factors can be examined.

There are basically two approaches for displaying the coloured 3D model (i.e. the 3D model on which the irradiance is applied). On the one hand, a static 3D model can be visualized for which the colours of the elements in the model do not change over time. It is rather useful, when the impact of different factors needs to be visualized (cf. FIG. 20A-20E; comparing also to the base 3D model of FIG. 20A itself). Static mode can also be used to examine how the manual adjustment of radiation positions affect the distribution of irradiance. In contrast, the dynamic mode highlights the irradiation process. It can be monitored how the number of properly irradiated surface elements increases as the time goes by (cf. FIG. 19A-19H; comparing also to the base 3D model of FIG. 19A itself).

It is relevant to note, that considering the genetic (evolutionary) algorithm it is adapted to the invention at least in two aspects: in the encoding of the individuals and the definition of the fitness function.

According to the above, it may be considered that a method (algorithm, software) is created which can simulate the irradiation process of an autonomous UV-C disinfection robot. We considered the main fundamental factors in the estimation of irradiance such as the distance from the light source, the angle of incidence and the time of irradiation. The proposed algorithm is capable of static and dynamic simulation and can be easily adjusted to different types of depth cameras and autonomous robots.

We further developed the above ray-tracing methodology by optimizing the movement of the robot along its path (radiation plan with discretized radiation positions) based on the data obtained from previous operation by using learning methods, like deep learning trained by previous paths, control instructions and searching plans.

As touched upon above, the robot learns semantic interpretation of the usual scenes. This helps it to predict (currently occluded) object's depths, normals, extension, etc. In this way on the hand the SLAM based navigation can be enhanced.

Summarizing from the point of view of the development, the method and system according to the invention can preferably estimate irradiance on nearby surfaces around a UV-C disinfection robot, and can also preferably simulate the irradiation process.

First, we built several synthetic worlds which served as test environments for the created application. Since the environment should be represented as an occupancy grid, in our experiments we reconstructed it using RTAB-Map. We designed also a measurement setup to determine the characteristics of a real UV-C lamp. Based on the acquired information on the lamp and the room to be disinfected, we could estimate irradiance for each voxel in the 3D model. We also developed a—for example genetic algorithm based—method/module to optimize the positions inside the room where the disinfection robot should stop to radiate. We highlighted above the features of the proposed system through static and dynamic simulations.

According to the above, the invention is, preferably, a self-learning, Simultaneous Localization and Mapping (SLAM) and ray-tracing analyser 3D geometric modelling for optimising the path planning and irradiation of UV-C disinfection robots.

The present invention is not limited to the preferred embodiments presented above, and further variants, modifications, changes, and improvements may also be conceived within the scope defined by the claims.

1. A method for generating a radiation plan of a scene in particular for a disinfection robot adapted for radiating disinfection light, in the course of the method
providing or generating a first 3D model (136, 310) with a plurality of 3D elements (242a-242g, 272) of a first scene (180, 200),
generating in a plan candidate generating step (S130, S161, S191) one or more plan candidate (112) of the radiation plan (135, 312) for the first 3D model (136, 310) by means of a first radiation plan generator module (130) based on artificial intelligence, wherein each of the one or more plan candidate (112) comprising
one or more radiation position (312a-312g), and
one or more respective radiation time each corresponding to a radiation position (312a-312g),
wherein a radiation position (312a-312g) and a corresponding radiation time constitute a radiation data pair,
generating in an irradiation evaluation step (S120, S162, S194) one or more respective irradiance ratio estimation (134) of the first 3D model (136, 310) for the one or more plan candidate (112) by evaluating the one or more plan candidate (112) by means of irradiation evaluation module (120), and
obtaining the radiation plan (135, 312) from the one or more plan candidate (112) based on the one or more respective irradiance ratio estimation (134) by means of the first radiation plan generator module (130) in a radiation plan choosing step (S168, S168′, S197, S226).

2. The method according to claim 1, characterized in that at least a part of the plurality of 3D elements (242a-242g, 272) of the 3D model (136, 310) constitutes investigation 3D elements having a number of investigation 3D elements, and in course of generating an irradiance ratio estimation (134) for a plan candidate (112) in the irradiation evaluation step (S120, S162, S194)
generating a respective one or more irradiance map of the investigation 3D elements by estimating an irradiance for each of the investigation 3D elements for each of the one or more radiation position (312a-312g) with the corresponding radiation time,
generating a fused irradiance map of the investigation 3D elements by summing up the one or more irradiance map of each of the one or more radiation positions (312a-312g) for all of the one or more radiation positions (312a-312g),
counting as a number of sufficiently irradiated 3D elements a number of the investigation 3D elements with irradiance exceeding a predetermined irradiance threshold from all of investigation 3D elements, and
obtaining the irradiance ratio estimation (134) as a ratio of the number of sufficiently irradiated 3D elements and the number of investigation 3D elements.

3. The method according to claim 2, characterized by taking into account a direct irradiance in the irradiation evaluation step (S120, S162, S194) for generating the respective irradiance ratio estimation (134) for each of the one or more plan candidate (112).

4. The method according to claim 3, characterized by calculating a direct irradiance increment in course of estimating the direct irradiance of each investigation 3D element for each radiation position (312a-312g) by investigating
whether a first distance between the investigation 3D element and the radiation position (312a-312g) is greater than a first predetermined distance limit (S152a),
in case of being greater, the direct irradiance increment for the investigation 3D element is set to zero (S123b),
in case of not being greater, by investigating (S154a) whether an angle of incidence of the direct irradiance is equal to zero,
in case of being equal to zero, the direct irradiance increment for the investigation 3D element is set to zero (S123b),
in case of not being equal to zero, by investigating (S156a) whether a radiation source characteristics parameter for the investigation 3D element has a value below a predetermined characteristics parameter limit,
in case of being below, the direct irradiance increment for the investigation 3D element is set to zero (S123b),
in case of not being below, by investigating (S158a) whether the investigation 3D element is visible from the radiation position (312a-312g),
in case of not being visible, the direct irradiance increment for the investigation 3D element is set to zero (S123b),
in case of being visible, the direct irradiance increment of the investigation 3D element is estimated based on an irradiance estimation formula (S159a).

5. The method according to claim 3, characterized by further taking into account an indirect irradiance in the irradiation evaluation step (S120, S162, S194) for generating the respective irradiance ratio estimation (134) for each of the one or more plan candidate (112).

6. The method according to claim 5, characterized by calculating an indirect irradiance increment in course of estimating the indirect irradiance of each investigation 3D element constituting a direct target 3D element (268) for each radiation position (312a-312g) by modelling at least one reflected ray (266), choosing respective at least one indirect target 3D element (270) based on an incoming ray (264) falling onto the direct target 3D element (268) from the radiation position (312a-312g) and investigating for each of the at least one indirect target 3D element (270)
whether a second distance between the direct target 3D element (268) and the indirect target 3D element (270) is greater than a second predetermined distance limit,
in case of being greater, the indirect irradiance increment for the indirect target 3D element (270) is set to zero,
in case of not being greater, by investigating whether an angle of incidence of the reflected ray (266) onto the indirect target 3D element (270) is equal to zero,
in case of being equal to zero, the indirect irradiance increment for the indirect target 3D element (270) is set to zero,
in case of not being equal to zero, by investigating whether a reflection parameter of the direct target 3D element (268) has a value below a predetermined reflection parameter limit,
in case of being below, the indirect irradiance increment for the indirect target 3D element (270) is set to zero,
in case of not being below, by investigating whether the indirect target 3D element (270) is visible from the direct target 3D element (268),
in case of not being visible, the indirect irradiance increment for the indirect target 3D element (270) is set to zero,
in case of being visible, the indirect irradiance increment of the indirect target 3D element (270) is estimated based on an irradiance estimation formula (S159b).

7. The method according to claim 1, characterized in that applying in the radiation plan choosing step (S168, S168′, S197) a radiation plan sufficiency criterion (S164a, S172, S196) being based on
a radiation time limit, whereby a summation of the one or more radiation time of the plan candidate (112) is intended to be equal to or below the radiation time limit, or
an irradiance limit, whereby the irradiance ratio estimation (134) of the plan candidate (112) is intended to be equal to or above the irradiance limit.

8. The method according to claim 7, characterized in that the first radiation plan generator module (130) is established by means of an evolutionary algorithm, at least two plan candidates (112) are generated in the plan candidate generating step (S161, S161′), each of the at least two plan candidates (112) has the same radiation data pair number of radiation data pair, a total radiation time is obtained for each plan candidate (112) by summing up the corresponding one or more radiation time for all of the one or more radiation position, and a first condition of the radiation plan sufficiency criterion (S164a) is applied in an irradiation iteration having at least one irradiation iteration cycle (S142, S145), wherein
in case of fulfilling the first condition,
the first radiation plan (135, 312) is obtained in the radiation plan choosing step (S168, S168′),
in case the radiation plan sufficiency criterion is based on the radiation time limit, from the at least two plan candidates (112) having a total radiation time below or equal to the radiation time limit, as the plan candidate (112) having the highest irradiance ratio estimation, and
in case the radiation plan sufficiency criterion is based on the irradiance limit, from the at least two plan candidates (112) having an irradiance ratio estimation (134) above or equal to the irradiance limit, as the plan candidate (112) having the lowest total radiation time, or
in case of fulfilling the first condition in the first of the at least one iteration cycle, the radiation data pair number is decreased by one, if it is above one, and the irradiation iteration is restarted.

9. The method according to claim 8, characterized in that applying a second condition of reaching of a predetermined maximum iteration number (S164b) in case the first condition is not fulfilled, wherein in case of fulfilling the second condition
the first radiation plan (135, 312) is obtained in the radiation plan choosing step (S168, S168′) as the plan candidate (112) having the highest efficiency proportion of the irradiance ratio estimation and the total radiation time, or
the radiation data pair number is increased by one and the irradiation iteration is restarted.

10. The method according to claim 9, characterized in that after generating the at least two plan candidates (112) in the plan candidate generating step (S161), in course of an iteration cycle (S142) of a first irradiation iteration
checking (S164) in the radiation plan choosing step (S168) a fulfilment of the first condition for the at least two plan candidates (112) and, if the first condition is not fulfilled, a fulfilment of the second condition,
in case the first condition or the second condition is fulfilled (S141a), the first radiation plan (135, 312) is obtained in the radiation plan choosing step (S168),
in case neither the first condition nor the second condition is fulfilled (S141b), at least two plan candidates (112) of a next iteration cycle (S142) are generated through an arbitrary order application of recombination (S165) and/or mutation (S166) of the at least two plan candidates (112),
wherein a respective irradiance ratio estimation (134) in the irradiation evaluation step (S162) is generated for each of the at least two plan candidates (112) during each iteration cycle (S142).

11. The method according to claim 10, characterized in that
the evolutionary algorithm is a genetic algorithm, the recombination is a crossover and the mutation is a mutation according to the genetic algorithm, and
in course of the first of the at least one iteration cycle (S142) the steps are ordered as
generating a respective irradiance ratio estimation (134) in the irradiation evaluation step (S162) for each of the at least two plan candidates (112), and
checking (S164) a fulfilment of the first condition and, if the first condition is not fulfilled, a fulfilment of the second condition.

12. The method according to claim 10, characterized in that
the evolutionary algorithm is a bacterial evolutionary algorithm, the recombination is a gene transfer and the mutation is a bacterial mutation, and
the respective irradiance ratio estimation (134) in the irradiation evaluation step (S162) is generated for each of the at least two plan candidates (112) within the gene transfer and/or the bacterial mutation.

13. The method according to claim 9, characterized in that the first radiation plan generator module (130) is established by means of a genetic algorithm, and, in course of an iteration cycle (S145) of a second irradiation iteration
generating at least two plan candidates (112) in the plan candidate generating step (S161′) as a first iteration starting step,
generating a respective irradiance ratio estimation (134) in the irradiation evaluation step (S162′) as a second iteration starting step for each of the at least two plan candidates (112),
checking a fulfilment of the first condition of the radiation plan sufficiency criterion (S172) for the at least two plan candidates (112),
in case the first condition is fulfilled (S143a), the first radiation plan is obtained in the radiation plan choosing step (S168′),
in case the first condition is not fulfilled (S143b), checking a fulfilment of the second condition,
in case
the second condition is fulfilled (S144a) and,
when the second condition is fulfilled for at least the second time, a first auxiliary proportion of
an actual highest efficiency proportion of the irradiance ratio estimation and the total radiation time of the at least two plan candidates (112) and
the previous first efficiency proportion parameter is above a first predetermined development parameter,
the highest efficiency proportion of the irradiance ratio estimation and the total radiation time of the at least two plan candidates (112) is given to a previous first efficiency proportion parameter and the radiation data pair number is increased by one for a next iteration cycle (S145) continued at the first iteration starting step (S145a),
in case
the second condition is fulfilled (S144a) and,
when the second condition is fulfilled for at least the second time,
the first auxiliary proportion of
an actual highest efficiency proportion of the irradiance ratio estimation and the total radiation time of the at least two plan candidates (112) and
the previous first efficiency proportion parameter is below the first predetermined development parameter,
the first radiation plan is obtained as the plan candidate (112) having the highest efficiency proportion of the irradiance ratio estimation and the total radiation time,
in case the second condition is not fulfilled (S144b), the at least two plan candidates (112) are generated for a next iteration cycle (S145) continued at the second iteration starting step (S145b) through crossover (S165′) and/or mutation (S166′) according to the genetic algorithm of the at least two plan candidates (112).

14. The method according to claim 7, characterized in that the first radiation plan generator module (130) is based on an incremental path generation algorithm, and, after generating the one or more plan candidate (112) in the plan candidate generating step (S191), in course of an iteration cycle (S147) of a third irradiation iteration
generating (S192a) cloned plan candidates for each plan candidate of the one or more plan candidates,
generating (S192b) a plurality of extended plan candidates by extending each of the cloned plan candidates with an additional radiation data pair,
generating a respective irradiance ratio estimation (134) in the irradiation evaluation step (S194) for each of the plurality of extended plan candidates,
checking a fulfilment of a first condition of the radiation plan sufficiency criterion (S196) for the plurality of extended plan candidates, wherein
in case the first condition is fulfilled (S146a), the first radiation plan is obtained in the radiation plan choosing step (S197) based on the plurality of the extended plan candidates,
in case
the first condition is not fulfilled (S146b) and,
when the first condition is not fulfilled for at least the second time, a second auxiliary proportion of
an actual highest efficiency proportion of the irradiance ratio estimation and the total radiation time of the plurality of extended plan candidates and
the previous second efficiency proportion parameter is above a second predetermined development parameter,
the highest efficiency proportion of the irradiance ratio estimation and the total radiation time of the plurality of extended plan candidates is given to a previous second efficiency proportion parameter and the one or more plan candidates (112) for a next iteration cycle (S147) is constituted by at least a part of the plurality of extended plan candidates, wherein a total radiation time is obtained for each plan candidate (112) by summing up the corresponding one or more radiation time for all of the one or more radiation position,
in case
the first condition is not fulfilled (S144a) and,
when the first condition is not fulfilled for at least the second time, the second auxiliary proportion of
an actual highest efficiency proportion of the irradiance ratio estimation and the total radiation time of the plurality of extended plan candidates and
the previous second efficiency proportion parameter is below the second predetermined development parameter,
the first radiation plan is obtained as the extended plan candidate having the highest efficiency proportion of the irradiance ratio estimation and the total radiation time.

15. The method according to claim 1, characterized in that the first radiation plan generator module (130) is based on a reinforcement learning algorithm, and in course of the method,
generating (S221) in a radiation data pair generating step a plurality of radiation data pairs,
in a starting radiation data pair assignment step after the radiation data pair generating step
generating the one or more plan candidate (112) in the radiation plan generating step by selecting one or more radiation data pair as respective one or more starting radiation data pair of a plan candidate,
generating a respective irradiance ratio estimation (134) in the irradiation evaluation step for the one or more starting radiation data pair and assigning (S222) a prize parameter value to each of the one or more starting radiation data pair based on the irradiance ratio estimation (134),
in an exploration step (S224) after the starting radiation data pair assignment step until a prize parameter value is assigned to each of the plurality of radiation data pairs, in course of an iteration cycle of a fourth irradiation iteration
generating one or more exploration plan candidate by extending a corresponding previous plan candidate having a previous irradiance ratio estimation with a respective additional radiation data pair,
generating a respective exploration irradiance ratio estimation in the irradiation evaluation step for each of the one or more exploration plan candidate, and
assigning a prize parameter value to each additional radiation data pair based on the difference between the exploration irradiance ratio estimation of the corresponding exploration plan candidate and the previous irradiance ratio estimation of the previous plan candidate, and
in an exploitation step (S225) after the exploration step (S224)
generating one or more exploitation plan candidate from the radiation data pairs based on the respective prize parameter values assigned thereto,
generating a respective exploitation irradiance ratio estimation in the irradiation evaluation step for each of the one or more exploitation plan candidate, and
in the radiation plan choosing step (S226) after the exploitation step (S225) the first radiation plan is obtained based on the exploitation plan candidate having
the highest exploitation irradiance ratio estimation, or
the lowest total radiation time, wherein total radiation time is obtained for each plan candidate by summing up the corresponding one or more radiation time for all of the one or more radiation position, or
the highest efficiency proportion of the irradiance ratio estimation and the total radiation time.

16. A system for generating a radiation plan of a scene in particular for a disinfection robot adapted for radiating disinfection light, the system comprising
a first radiation plan generator module (130) based on artificial intelligence and adapted for generating one or more plan candidate (112) of the radiation plan (135, 312) for the first 3D model (136, 310) with a plurality of 3D elements (242a-242g, 272) of a first scene (180, 200), wherein each of the one or more plan candidate (112) comprising
one or more radiation position (312a-312g), and
one or more respective radiation time each corresponding to a radiation position (312a-312g),
wherein a radiation position (312a-312g) and a corresponding radiation time constitute a radiation data pair, and
an irradiation evaluation module (120) adapted for generating a respective irradiance ratio estimation (134) of the first 3D model (136, 310) by evaluating the one or more plan candidate (112),
wherein the first radiation plan generator module (130) is furthermore adapted for obtaining the radiation plan (135, 312) from the one or more plan candidate (112) based on the one or more respective irradiance ratio estimation (134).

17. A method for generating a radiation plan of a scene in particular for a disinfection robot adapted for radiating disinfection light, in the course of which
providing or generating a 2D or 3D scene representation of a second scene (200), and
generating (S245) a radiation plan for the 2D or 3D scene representation by means of a second radiation plan generator module based on deep learning trained (S244) by a plurality of first radiation plans of respective first scenes generated (S242) by the method of claim 1.

18. A system for generating a radiation plan of a scene in particular for a disinfection robot adapted for radiating disinfection light, the system comprising
a second radiation plan generator module adapted for generating (S245) a radiation plan for a 2D or 3D scene representation of a second scene (200) and based on deep learning trained (S244) by a plurality of first radiation plans of respective first scenes generated (S242) by the method of claim 1.
